(19) United States
(12) Patent Application Publication
(10) Pub. No.: US 2022/0328769 A1
(43) Pub. Date: Oct. 13, 2022

(54) HETEROCYCLIC COMPOUND AND ORGANIC LIGHT-EMITTING DEVICE COMPRISING SAME

(71) Applicant: LT MATERIALS CO., LTD., Yongin-si, Gyeonggi-do (KR)

(72) Inventors: Hye-Su JI, Yongin-si (KR); Dong-Jun KIM, Yongin-si (KR); Hyun-Joo LEE, Yongin-si (KR); Young-Seok NO, Yongin-si (KR)

(73) Assignee: LT MATERIALS CO., LTD., Yongin-si, Gyeonggi-do (KR)

(21) Appl. No.: 17/608,831

(22) PCT Filed: Nov. 5, 2020
(86) PCT No.: PCT/KR2020/015381

§ 371 Date: Nov. 4, 2021

(30) Foreign Application Priority Data
Nov. 5, 2019 (KR) 10-2019-0140381

(51) Int. Cl. H01L 51/00; C07D 405/14; C09K 11/06; C07D 411/14; C07D 413/14; C07F 7/08; C07D 405/04; C07D 417/14

(52) U.S. Cl. CPC H01L 51/0073; C07D 405/14; C09K 11/06; H01L 51/0067; C07D 411/14; H01L 51/0071; C07D 413/14; C07F 7/0816; H01L 51/0094; C07D 405/04; H01L 51/0052; C07D 417/14; H01L 51/0074; H01L 51/0072; H01L 51/5016

(57) ABSTRACT

The present specification relates to a heterocyclic compound represented by Chemical Formula 1, and an organic light emitting device comprising the same.

### TECHNICAL FIELD

This application claims priority to and the benefits of Korean Patent Application No. 10-2019-0140381, filed with the Korean Intellectual Property Office on Nov. 5, 2019, the entire contents of which are incorporated herein by reference. The present specification relates to a heterocyclic compound, and an organic light emitting device comprising the same.

### BACKGROUND ART

An organic electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

U.S. Pat. No. 4,356,429

### DISCLOSURE

### Technical Problem

One embodiment of the present application relates to a heterocyclic compound represented by Chemical Formula 1, and an organic light emitting device comprising the same.

### Technical Solution

One embodiment of the present application provides a heterocyclic compound represented by the following Chemical Formula 1.

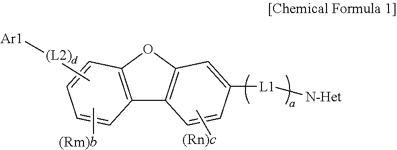

In Chemical Formula 1,

N-Het is a monocyclic or polycyclic C2 to C60 heterocyclic group substituted or unsubstituted, and comprising one or more Ns,

L1 and L2 are a direct bond; a substituted or unsubstituted C6 to C60 arylene group; or a substituted or unsubstituted C2 to C60 heteroarylene group, a and d are each an integer of 1 to 3, and when a is 2 or greater, L1s are the same as or different from each other, and when d is 2 or greater, L2s are the same as or different from each other,

Rm and Rn are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; and —NRR′, or two or more groups adjacent to each other bond to each other to form a substituted or unsubstituted C6 to C60 aliphatic or aromatic hydrocarbon ring or C2 to C60 heteroring, b and c are each an integer of 1 to 3, and when b is 2 or greater, Rms are the same as or different from each other, and when c is 2 or greater, Rns are the same as or different from each other, and

Ar1 is represented by the following Chemical Formula 2 or 3,

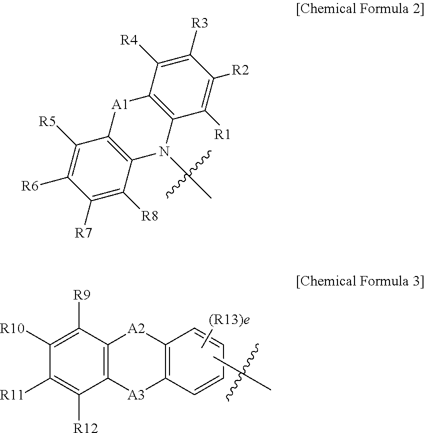

in Chemical Formulae 2 and 3,

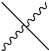

means a site linked to L2 of Chemical Formula 1,

R1 to R13 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; and —NRR′, or two or more groups adjacent to each other bond to each other to form a substituted or unsubstituted C6 to C60 aliphatic or aromatic hydrocarbon ring or C2 to C60 heteroring, e is an integer of 1 to 3, and when e is 2 or greater, R13s are the same as or different from each other,

A1 and A2 are the same as or different from each other, and each independently O; S; CRaRb; NRc; or SiRdRe,

A3 is a direct bond; O; S; CRaRb; NRc; or SiRdRe, and

R, R′ and Ra to Re are the same as or different from each other, and each independently hydrogen; a substituted or unsubstituted C1 to C60 alkyl group; a substituted or unsubstituted C6 to C60 aryl group; or a substituted or unsubstituted C2 to C60 heteroaryl group, or two or more groups adjacent to each other bond to each other to form a substituted or unsubstituted C6 to C60 aliphatic or aromatic hydrocarbon ring or C2 to C60 heteroring.

Another embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound represented by Chemical Formula 1.

### Advantageous Effects

A compound described in the present specification can be used as a material of an organic material layer of an organic light emitting device. In the organic light emitting device, the compound is capable of performing a role of a hole injection material, a hole transfer material, a light emitting material, an electron transfer material, an electron injection material or the like. Particularly, the compound can be used as a light emitting layer material of the organic light emitting device. For example, the compound can be used as a light emitting material alone, or can be used as a host material of the light emitting layer.

Particularly, by substituting a position of No. 3 carbon of a dibenzofuran structure with an N-containing ring and substituting benzene not substituted with the N-containing ring in the dibenzofuran ring with a substituent represented by Chemical Formula 2 or 3, Chemical Formula 1 has a more electron-stable structure, and as a result, a device lifetime can be enhanced.

In addition, by Ar of Chemical Formula 1 being represented by Chemical Formula 2 or Chemical Formula 3, electron distribution spreads widely from the dibenzofuran core to the substituents of Ar resulting in a wide band gap and a high T1 value. Accordingly, due to the properties of wide band gap and high T1 compared to cases of having carbazoles, an organic light emitting device having superior efficiency and low driving voltage is obtained in the present application when using Chemical Formula 1 as a phosphorescent host material of the organic light emitting device.

### DESCRIPTION OF DRAWINGS

FIG. 1 to FIG. 3 are diagrams each schematically illustrating a lamination structure of an organic light emitting device according to one embodiment of the present application.

### REFERENCE NUMERAL

100: Substrate
200: Anode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Cathode

### MODE FOR DISCLOSURE

Hereinafter, the present application will be described in detail.

In the present specification, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present application, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present application, in a “case of a substituent being not indicated in a chemical formula or compound structure”, hydrogen and deuterium may be mixed in compounds when deuterium is not explicitly excluded such as a deuterium content being 0% or a hydrogen content being 100%. In other words, an expression of “substituent X is hydrogen” does not exclude deuterium such as a hydrogen content being 100% or a deuterium content being 0%, and therefore, may mean a state in which hydrogen and deuterium are mixed.

In one embodiment of the present application, deuterium is one of isotopes of hydrogen, is an element having deuteron formed with one proton and one neutron as a nucleus, and may be expressed as hydrogen-2, and the elemental symbol may also be written as D or 2H.

In one embodiment of the present application, an isotope means an atom with the same atomic number (Z) but with a different mass number (A), and may also be interpreted as an element with the same number of protons but with a different number of neutrons.

In one embodiment of the present application, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

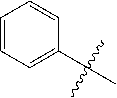 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulae.

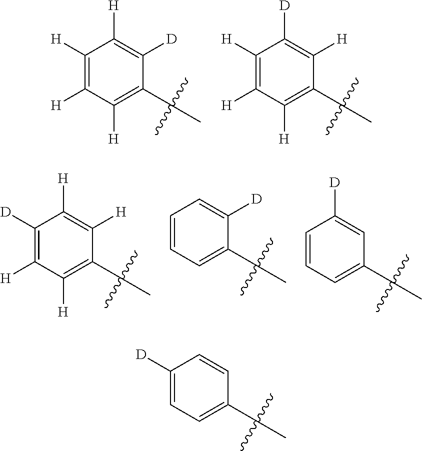 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present application, “a phenyl group having a deuterium content of 0%” may mean a phenyl group that does not comprise a deuterium atom, that is, a phenyl group that has 5 hydrogen atoms.

The term “substitution” means a hydrogen atom bonding to a carbon atom of a compound being changed to another substituent, and the position of substitution is not limited as long as it is a position at which the hydrogen atom is substituted, that is, a position at which a substituent can substitute, and when two or more substituents substitute, the two or more substituents may be the same as or different from each other.

In the present specification, “substituted or unsubstituted” means being substituted with one or more substituents selected from the group consisting of C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine, or being unsubstituted, or being substituted with a substituent linking two or more substituents selected from among the substituents illustrated above, or being unsubstituted.

In the present application, R, R′ and R″ are the same as or different from each other, and may be each independently hydrogen; a substituted or unsubstituted alkyl group; a substituted or unsubstituted aryl group; or a substituted or unsubstituted heteroaryl group.

In the present specification, the halogen may be fluorine, chlorine, bromine or iodine.

In the present specification, the alkyl group comprises linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may comprise a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group comprises linear or branched having 2 to 60 carbon atoms, and may be further substituted with other substituents. The number of carbon atoms of the alkenyl group may be from 2 to 60, specifically from 2 to 40 and more specifically from 2 to 20. Specific examples thereof may comprise a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group comprises linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may comprise methoxy, ethoxy, n-propoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like, but are not limited thereto.

In the present specification, the cycloalkyl group comprises monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may comprise a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group comprises O, S, Se, N or Si as a heteroatom, comprises monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group comprises monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group comprises a spiro group. The number of carbon atoms of the aryl group may be from 6 to 60, specifically from 6 to 40 and more specifically from 6 to 25. Specific examples of the aryl group may comprise a phenyl group, a biphenyl group, a triphenyl group (terphenyl group), a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring thereof, and the like, but are not limited thereto.

In the present specification, the fluorenyl group may be substituted, and adjacent substituents may bond to each other to form a ring.

When the fluorenyl group is substituted, the following structures may be included, however, the structure is not limited thereto.

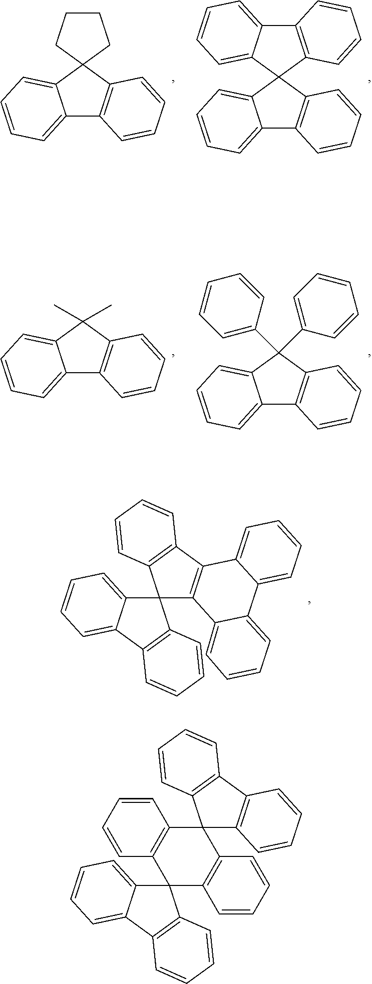

In the present specification, the heteroaryl group comprises S, O, Se, N or Si as a heteroatom, comprises monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may comprise a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrobenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may comprise a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, the phosphine oxide group is represented by —P(═O) R₁₀₁R₁₀₂, and R₁₀₁ and R₁₀₂ are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the phosphine oxide may comprise a diphenylphosphine oxide group, a dinaphthylphosphine oxide group and the like, but are not limited thereto.

In the present specification, the silyl group is a substituent comprising Si, having the Si atom directly linked as a radical, and is represented by —SiR₁₀₄R₁₀₅R₁₀₆. R₁₀₄ to R₁₀₆ are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may comprise a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

As the aliphatic or aromatic hydrocarbon ring or the heteroring that adjacent groups may form, the structures illustrated as the cycloalkyl group, the cycloheteroalkyl group, the aryl group and the heteroaryl group described above may be applied except for those that are not a monovalent group.

One embodiment of the present application provides a compound represented by Chemical Formula 1.

In one embodiment of the present application, Chemical Formula 1 may be represented by any one of the following Chemical Formulae 4 to 7.

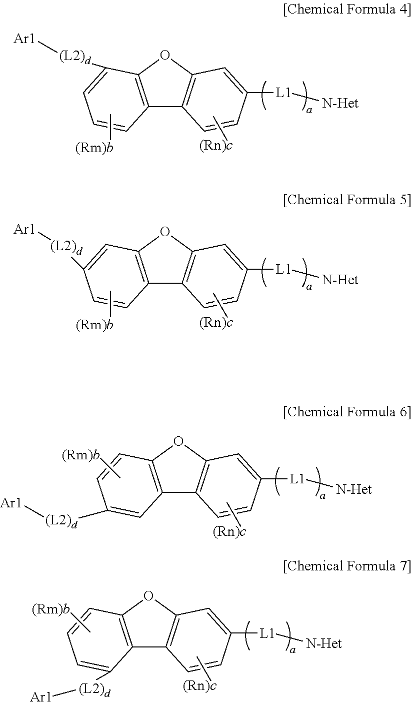

In Chemical Formulae 4 to 7,

each substituent has the same definition as in Chemical Formula 1.

In one embodiment of the present application, Rm and Rn are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; and —NRR′, or two or more groups adjacent to each other may bond to each other to form a substituted or unsubstituted C6 to C60 aliphatic or aromatic hydrocarbon ring or C2 to C60 heteroring.

In another embodiment, Rm and Rn are the same as or different from each other, and each independently selected from the group consisting of hydrogen; a substituted or unsubstituted C1 to C60 alkyl group; a substituted or unsubstituted C6 to C60 aryl group; and a substituted or unsubstituted C2 to C60 heteroaryl group, or two or more groups adjacent to each other may bond to each other to form a substituted or unsubstituted C6 to C60 aromatic hydrocarbon ring or C2 to C60 heteroring.

In another embodiment, Rm and Rn may be hydrogen.

In one embodiment of the present application, L1 and L2 may be a direct bond; a substituted or unsubstituted C6 to C60 arylene group; or a substituted or unsubstituted C2 to C60 heteroarylene group.

In another embodiment, L1 and L2 may be a direct bond; a substituted or unsubstituted C6 to C40 arylene group; or a substituted or unsubstituted C2 to C40 heteroarylene group.

In another embodiment, L1 and L2 may be a direct bond; or a substituted or unsubstituted C6 to C40 arylene group.

In another embodiment, L1 and L2 may be a direct bond; or a C6 to C40 arylene group.

In another embodiment, L1 and L2 may be a direct bond; or a C6 to C40 monocyclic arylene group.

In another embodiment, L1 and L2 may be a direct bond; or a C6 to C20 monocyclic arylene group.

In another embodiment, L1 and L2 may be a direct bond; or a phenylene group.

In one embodiment of the present application, N-Het may be a monocyclic or polycyclic C2 to C60 heterocyclic group substituted or unsubstituted, and comprising one or more Ns.

In another embodiment, N-Het may be a monocyclic or polycyclic C2 to C60 heterocyclic group substituted or unsubstituted, and comprising one or more and three or less Ns.

In another embodiment, N-Het may be a monocyclic C2 to C60 heterocyclic group substituted or unsubstituted, and comprising one or more and three or less Ns.

In another embodiment, N-Het may be a monocyclic C2 to C60 heterocyclic group unsubstituted or substituted with one or more substituents selected form the group consisting of a C6 to C60 aryl group and a C2 to C60 heteroaryl group, and comprising one or more and three or less Ns.

In another embodiment, N-Het may be a monocyclic C2 to C60 heterocyclic group unsubstituted or substituted with one or more substituents selected form the group consisting of a C6 to C40 aryl group and a C2 to C40 heteroaryl group, and comprising one or more and three or less Ns.

In another embodiment, N-Het may be a triazine group unsubstituted or substituted with one or more substituents selected form the group consisting of a C6 to C40 aryl group and a C2 to C40 heteroaryl group; a pyrimidine group unsubstituted or substituted with one or more substituents selected form the group consisting of a C6 to C40 aryl group and a C2 to C40 heteroaryl group; or a pyridine group unsubstituted or substituted with one or more substituents selected form the group consisting of a C6 to C40 aryl group and a C2 to C40 heteroaryl group.

In another embodiment, N-Het may be represented by the following Chemical Formula 8.

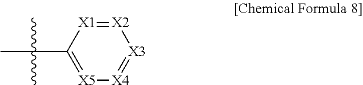

In Chemical Formula 8,

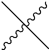

means a site linked to L1 of Chemical Formula 1,

X1 is CR21 or N, X2 is CR22 or N, X3 is CR23 or N, X4 is CR24 or N, and X5 is CR25 or N,

at least one of X1 to X5 is N, and

R21 to R25 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; and —NRR′, or two or more groups adjacent to each other bond to each other to form a substituted or unsubstituted C6 to C60 aliphatic or aromatic hydrocarbon ring or C2 to C60 heteroring.

In one embodiment of the present application, R21 to R25 are the same as or different from each other, and may be each independently hydrogen; a substituted or unsubstituted C6 to C60 aryl group; or a substituted or unsubstituted C2 to C60 heteroaryl group.

In another embodiment, R21 to R25 are the same as or different from each other, and may be each independently a substituted or unsubstituted C6 to C40 aryl group; or a substituted or unsubstituted C2 to C40 heteroaryl group.

In another embodiment, R21 to R25 are the same as or different from each other, and may be each independently a C6 to C40 aryl group unsubstituted or substituted with one or more substituents selected form the group consisting of a C6 to C40 aryl group and a C2 to C40 heteroaryl group; or a C2 to C40 heteroaryl group unsubstituted or substituted with one or more substituents selected form the group consisting of a C6 to C40 aryl group and a C2 to C40 heteroaryl group.

In another embodiment, R21 to R25 are the same as or different from each other, and may be each independently a C6 to C40 aryl group unsubstituted or substituted with a C2 to C40 heteroaryl group; or a C2 to C40 heteroaryl group unsubstituted or substituted with a C6 to C40 aryl group.

In another embodiment, R21 to R25 are the same as or different from each other, and may be each independently a phenyl group unsubstituted or substituted with a carbazole group; a biphenyl group; a naphthyl group; a carbazole group unsubstituted or substituted with a phenyl group; a dibenzofuran group; or a dibenzothiophene group.

In one embodiment of the present application, Chemical Formula 8 may be selected from among the following structural formulae.

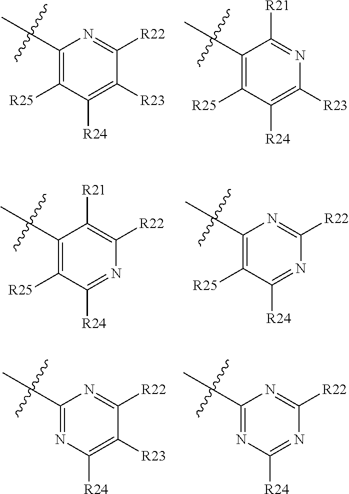

In the structural formulae,

R21 to R25 have the same definitions as in Chemical Formula 8.

In one embodiment of the present application, Ar may be represented by the following Chemical Formula 2 or 3.

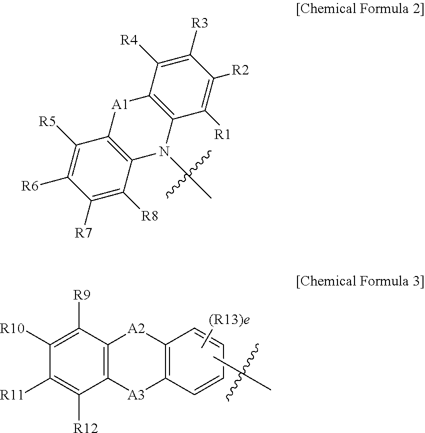

In Chemical Formulae 2 and 3,

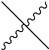

means a site linked to L2 of Chemical Formula 1,

R1 to R13 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; and —NRR′, or two or more groups adjacent to each other bond to each other to form a substituted or unsubstituted C6 to C60 aliphatic or aromatic hydrocarbon ring or C2 to C60 heteroring, e is an integer of 1 to 3, and when e is 2 or greater, R13s are the same as or different from each other,

A1 and A2 are the same as or different from each other, and each independently O; S; CRaRb; NRc; or SiRdRe,

A3 is a direct bond; O; S; CRaRb; NRc; or SiRdRe, and

R, R′ and Ra to Re are the same as or different from each other, and each independently hydrogen; a substituted or unsubstituted C1 to C60 alkyl group; a substituted or unsubstituted C6 to C60 aryl group; or a substituted or unsubstituted C2 to C60 heteroaryl group, or two or more groups adjacent to each other bond to each other to form a substituted or unsubstituted C6 to C60 aliphatic or aromatic hydrocarbon ring or C2 to C60 heteroring.

In one embodiment of the present application, R1 to R13 are the same as or different from each other, and each independently selected from the group consisting of hydrogen; a substituted or unsubstituted C1 to C60 alkyl group; a substituted or unsubstituted C6 to C60 aryl group; a substituted or unsubstituted C2 to C60 heteroaryl group; —P(═O)RR′; and —NRR′, or two or more groups adjacent to each other may bond to each other to form a substituted or unsubstituted C6 to C60 aliphatic or aromatic hydrocarbon ring or C2 to C60 heteroring.

In another embodiment, R1 to R13 are the same as or different from each other, and may be each independently selected from the group consisting of hydrogen; a substituted or unsubstituted C6 to C60 aryl group; and a substituted or unsubstituted C2 to C60 heteroaryl group.

In another embodiment, R1 to R13 are the same as or different from each other, and may be each independently selected from the group consisting of hydrogen; a C6 to C60 aryl group; and a C2 to C60 heteroaryl group.

In another embodiment, R1 to R13 may be hydrogen.

In one embodiment of the present application, A1 and A2 are the same as or different from each other, and may be each independently O; S; CRaRb; NRc; or SiRdRe.

In one embodiment of the present application, A3 may be a direct bond; O; S; CRaRb; NRc; or SiRdRe.

In one embodiment of the present application, Ra to Re are the same as or different from each other, and each independently hydrogen; a substituted or unsubstituted C1 to C60 alkyl group; a substituted or unsubstituted C6 to C60 aryl group; or a substituted or unsubstituted C2 to C60 heteroaryl group, or two or more groups adjacent to each other may bond to each other to form a substituted or unsubstituted C6 to C60 aliphatic or aromatic hydrocarbon ring or C2 to C60 heteroring.

In another embodiment, Ra to Re are the same as or different from each other, and each independently hydrogen; a substituted or unsubstituted C1 to C60 alkyl group; a substituted or unsubstituted C6 to C60 aryl group; or a substituted or unsubstituted C2 to C60 heteroaryl group, or two or more groups adjacent to each other may bond to each other to form a substituted or unsubstituted C6 to C60 aromatic hydrocarbon ring or C2 to C60 heteroring.

In another embodiment, Ra to Re are the same as or different from each other, and each independently hydrogen; a substituted or unsubstituted C1 to C40 alkyl group; a substituted or unsubstituted C6 to C40 aryl group; or a substituted or unsubstituted C2 to C40 heteroaryl group, or two or more groups adjacent to each other may bond to each other to form a substituted or unsubstituted C6 to C40 aromatic hydrocarbon ring or C2 to C40 heteroring.

In another embodiment, Ra to Re are the same as or different from each other, and each independently a C1 to C40 alkyl group; or a C6 to C40 aryl group, or two or more groups adjacent to each other may bond to each other to form a C6 to C40 aromatic hydrocarbon ring or C2 to C40 heteroring.

In another embodiment, Ra to Re are the same as or different from each other, and each independently a methyl group; or a phenyl group, or two or more groups adjacent to each other may bond to each other to form a xanthene ring; a fluorene ring; or a 9,10-dihydroanthracene ring.

In one embodiment of the present application, when A3 is a direct bond, A2 may be CRaRb, and Ra and Rb may bond to each other to form a xanthene ring.

In one embodiment of the present application, Chemical Formula 3 may be represented by the following Chemical Formulae 3-1 to 3-4.

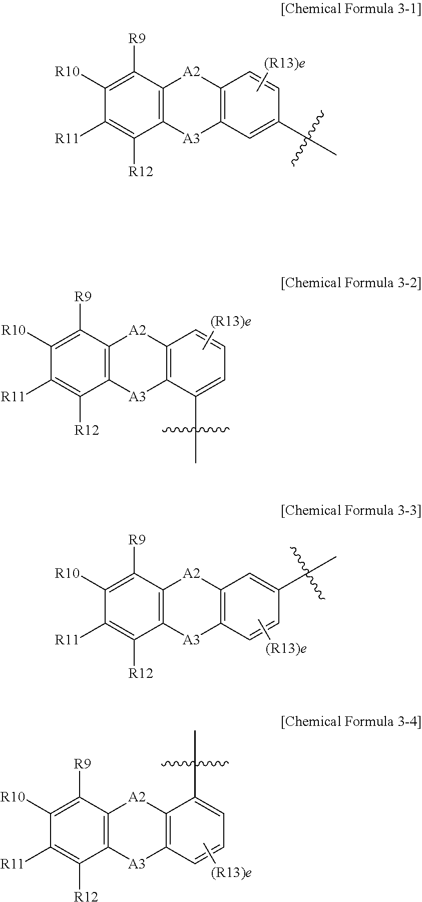

In Chemical Formulae 3-1 to 3-4,

each substituent has the same definition as in Chemical Formula 3.

In one embodiment of the present application, by Ar of Chemical Formula 1 being represented by Chemical Formula 2 or Chemical Formula 3, electron distribution spreads widely from the dibenzofuran core to the substituents of Ar resulting in a wide band gap and a high T1 value. In addition, by Ar of Chemical Formula 1 having the substituents of Chemical Formula 2 or Chemical Formula 3, a stable molecular structure may be maintained contributing to enhancement in the lifetime.

Accordingly, due to the properties of wide band gap and high T1 compared to cases of having carbazoles, an organic light emitting device having superior efficiency and low driving voltage is obtained in the present application when using Chemical Formula 1 as a phosphorescent host material of the organic light emitting device.

According to one embodiment of the present application, Chemical Formula 1 may be represented by any one of the following compounds, but is not limited thereto.

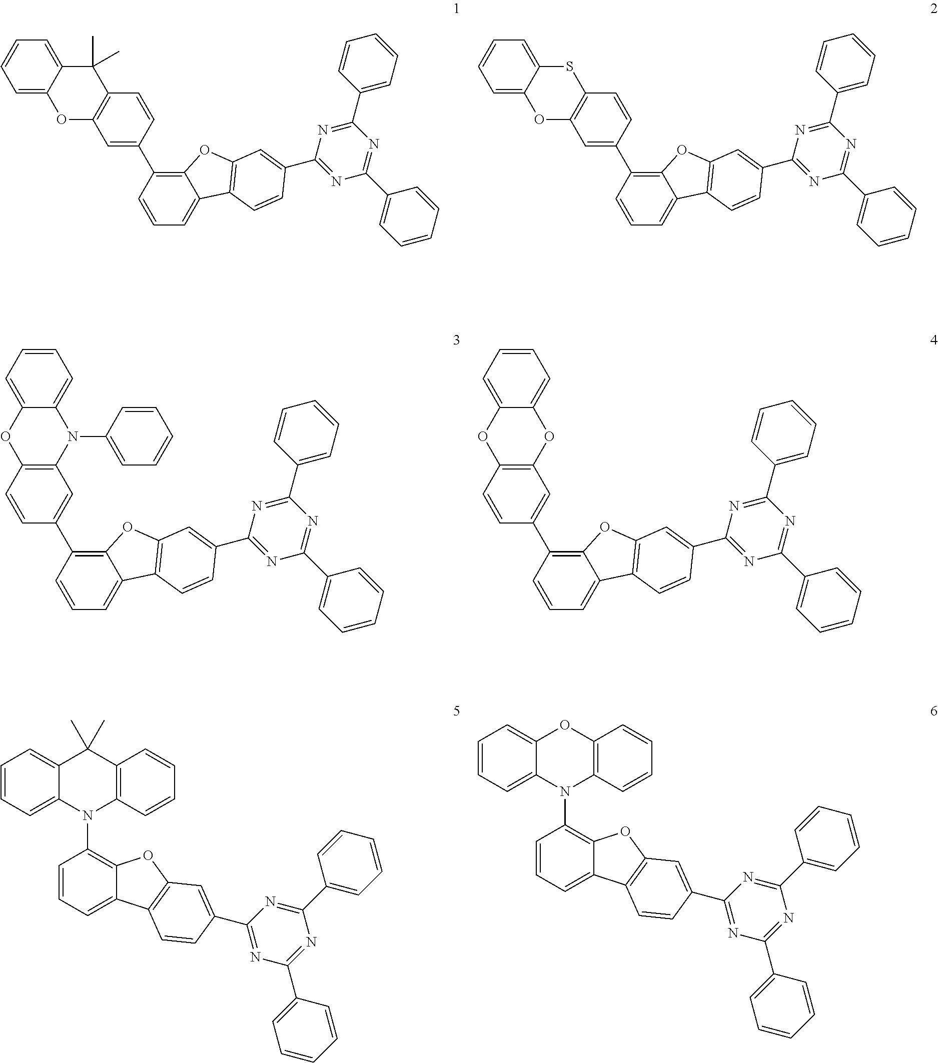 [SMILES: CC1(C)c2ccccc2Oc2cc(-c3cccc4c3oc3cc(-c5nc(-c6ccccc6)nc(-c6ccccc6)n5)ccc34)ccc21 ; c1ccc(-c2nc(-c3ccccc3)nc(-c3ccc4c(c3)oc3c(-c5ccc6c(c5)Oc5ccccc5S6)cccc34)n2)cc1 ; c1ccc(-c2nc(-c3ccccc3)nc(-c3ccc4c(c3)oc3c(-c5ccc6c(c5)N(c5ccccc5)c5ccccc5O6)cccc34)n2)cc1 ; c1ccc(-c2nc(-c3ccccc3)nc(-c3ccc4c(c3)oc3c(-c5ccc6c(c5)Oc5ccccc5O6)cccc34)n2)cc1 ; CC1(C)c2ccccc2N(c2cccc3c2oc2cc(-c4nc(-c5ccccc5)nc(-c5ccccc5)n4)ccc23)c2ccccc21 ; c1ccc(-c2nc(-c3ccccc3)nc(-c3ccc4c(c3)oc3c(N5c6ccccc6Oc6ccccc65)cccc34)n2)cc1]

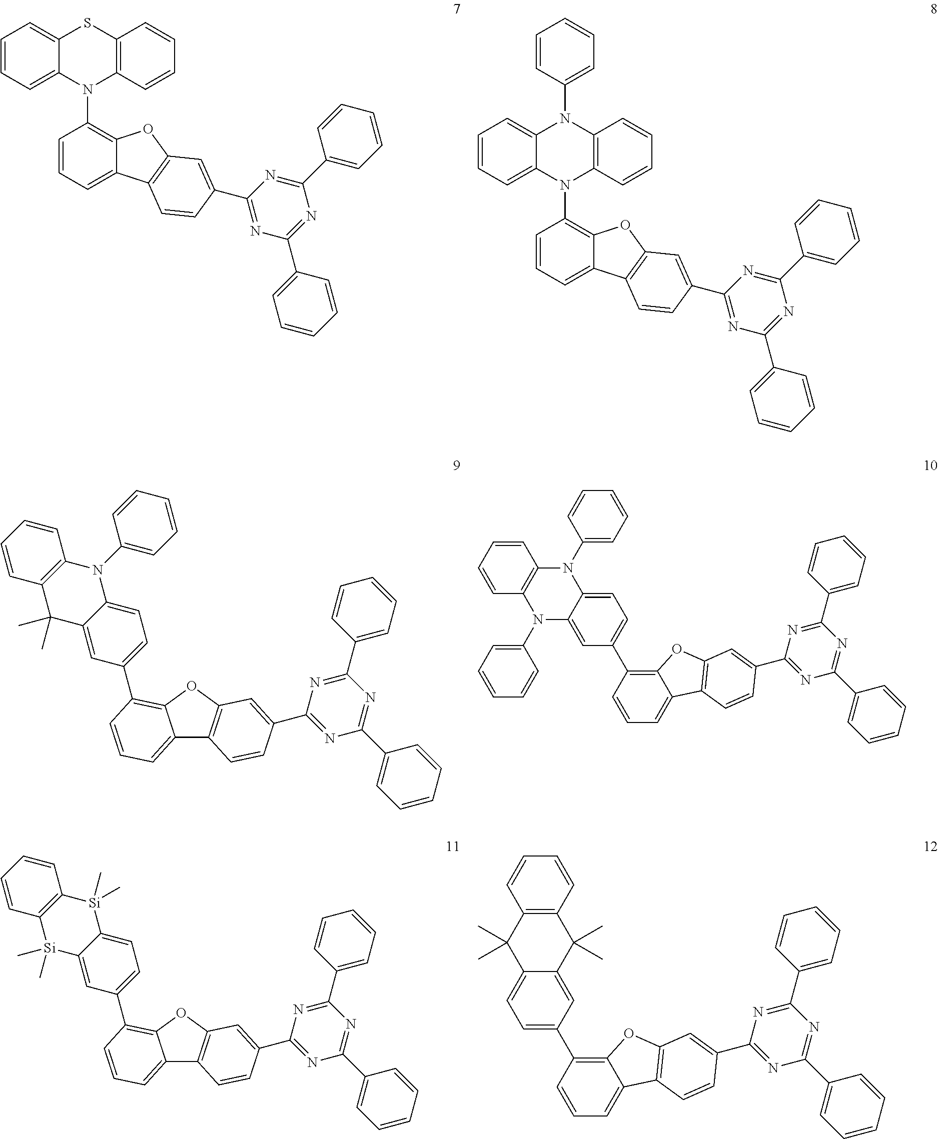

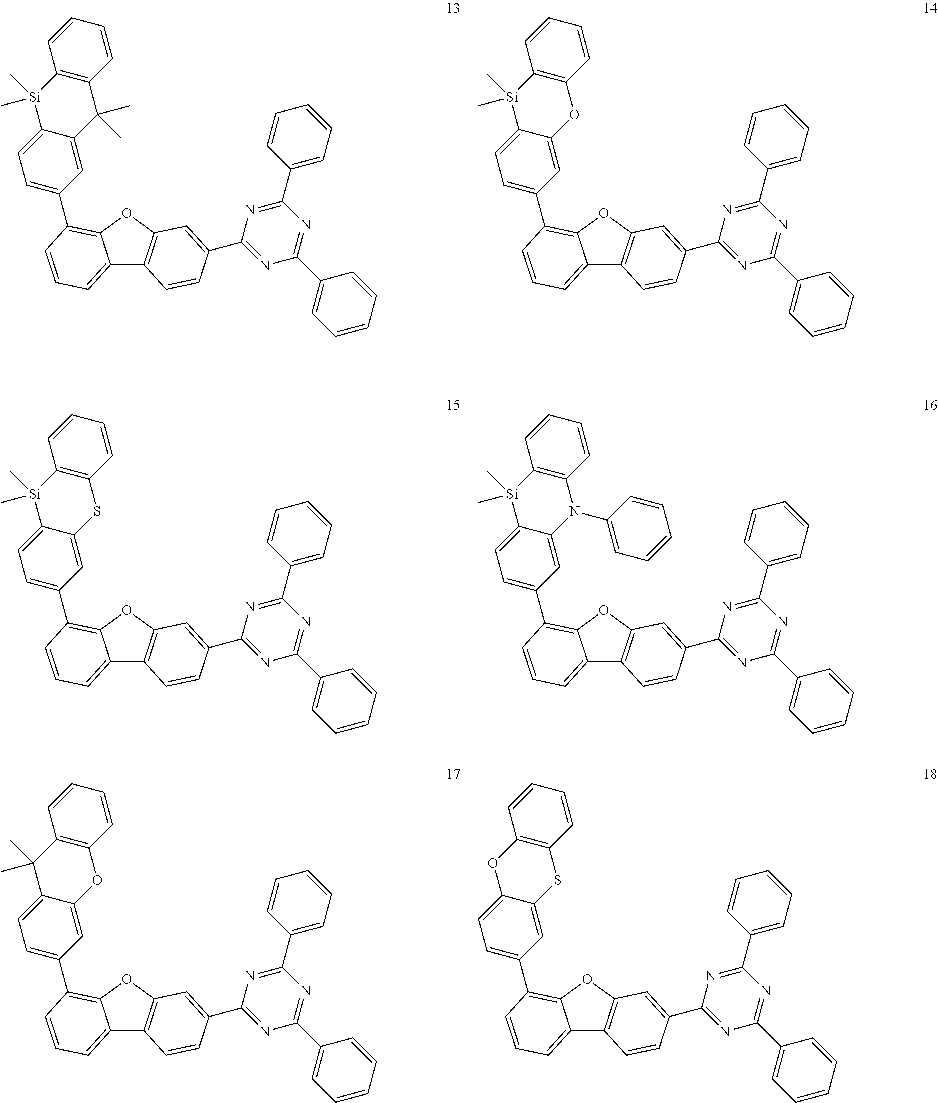

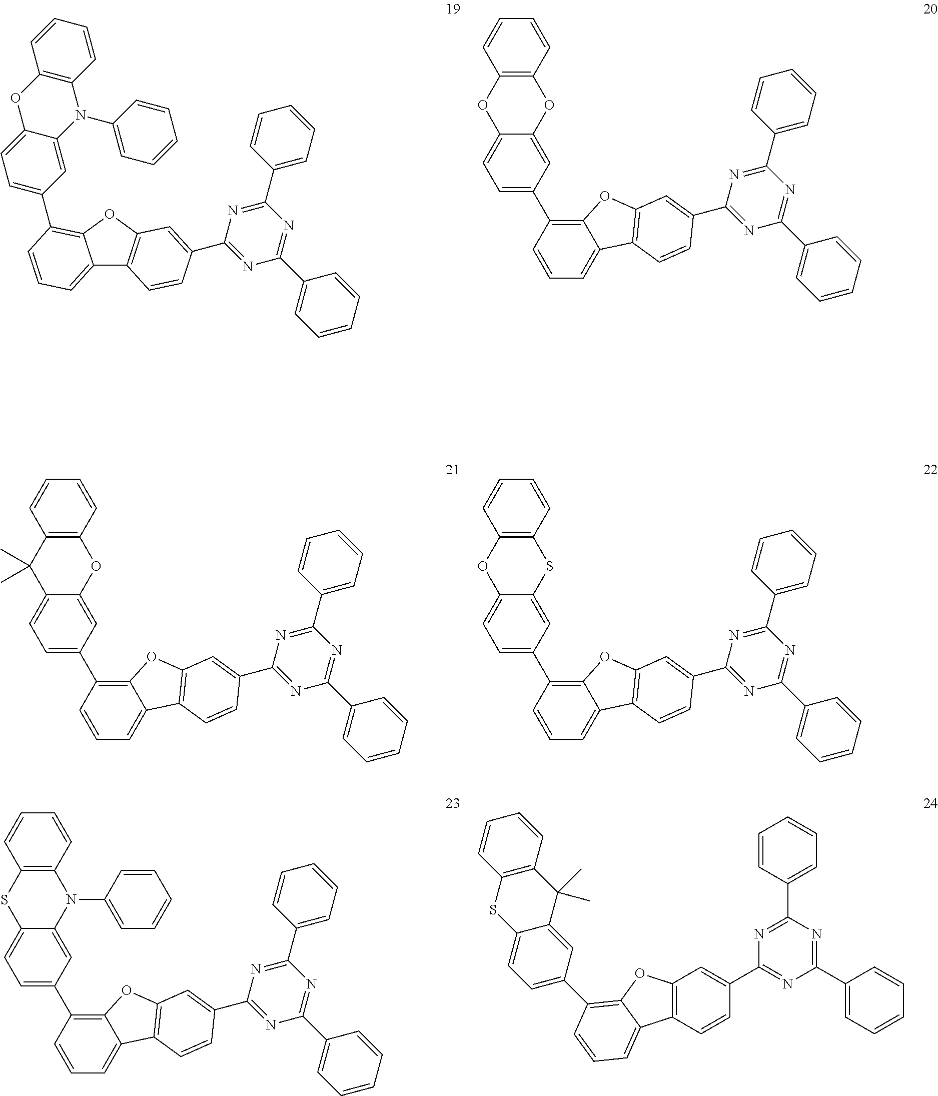

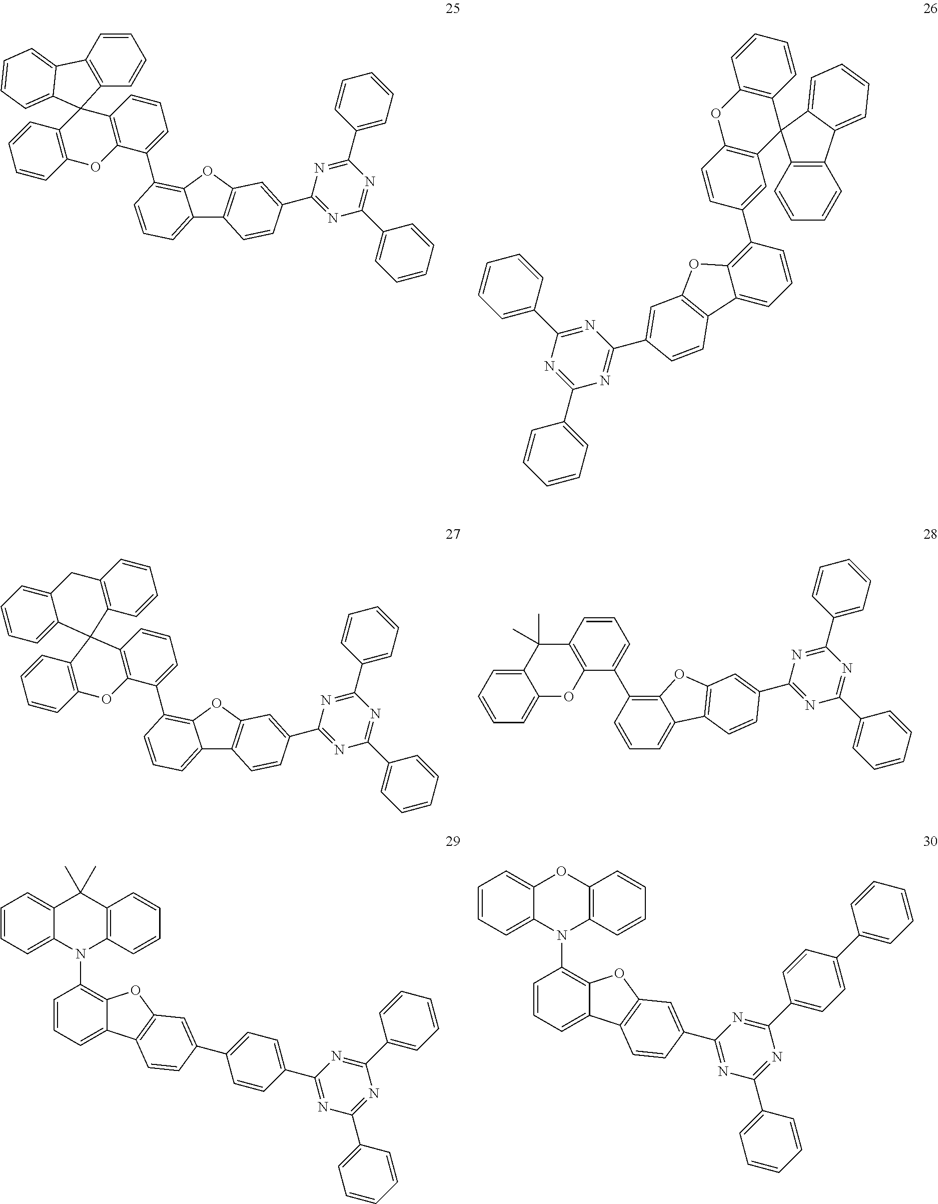

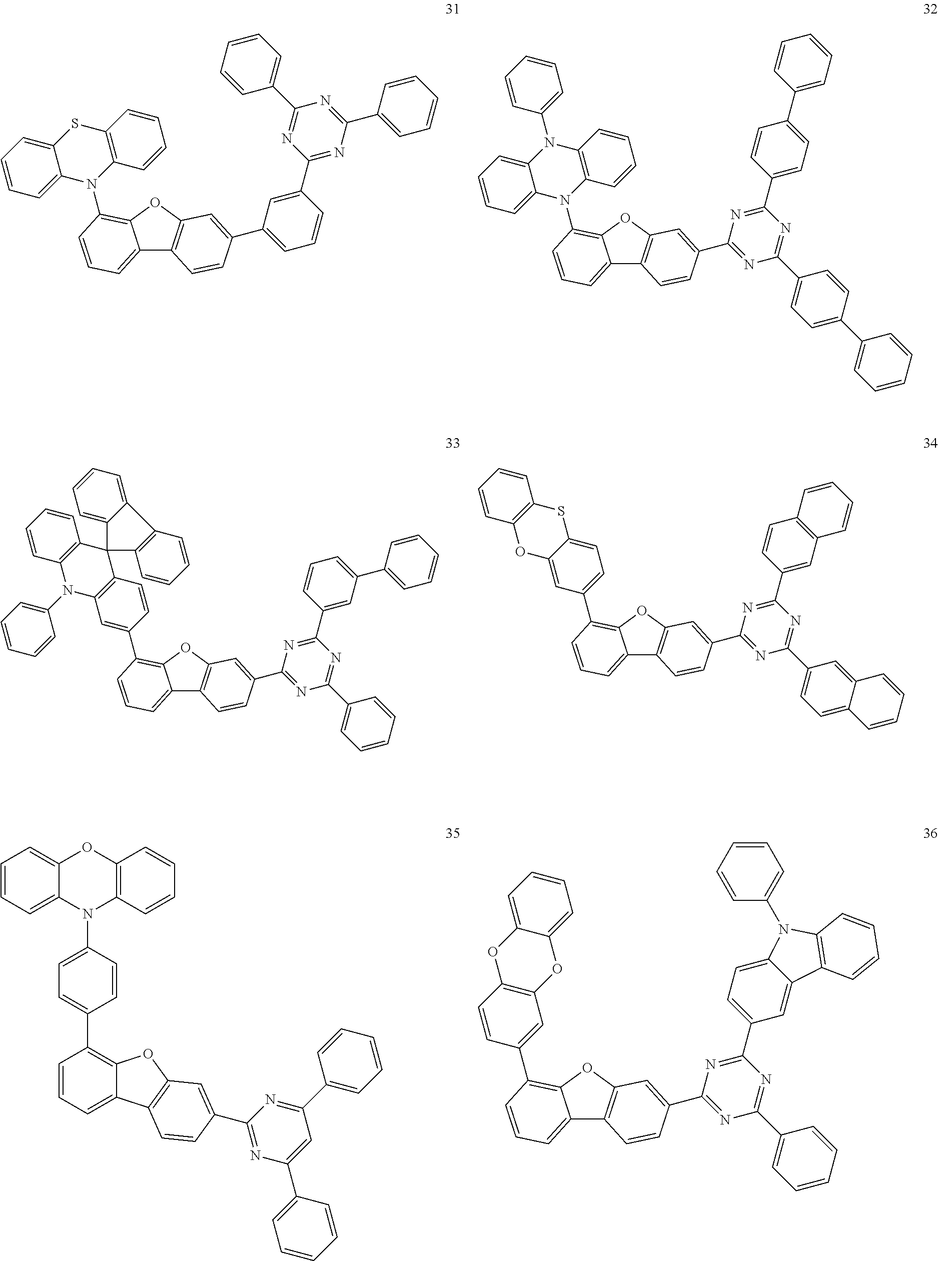

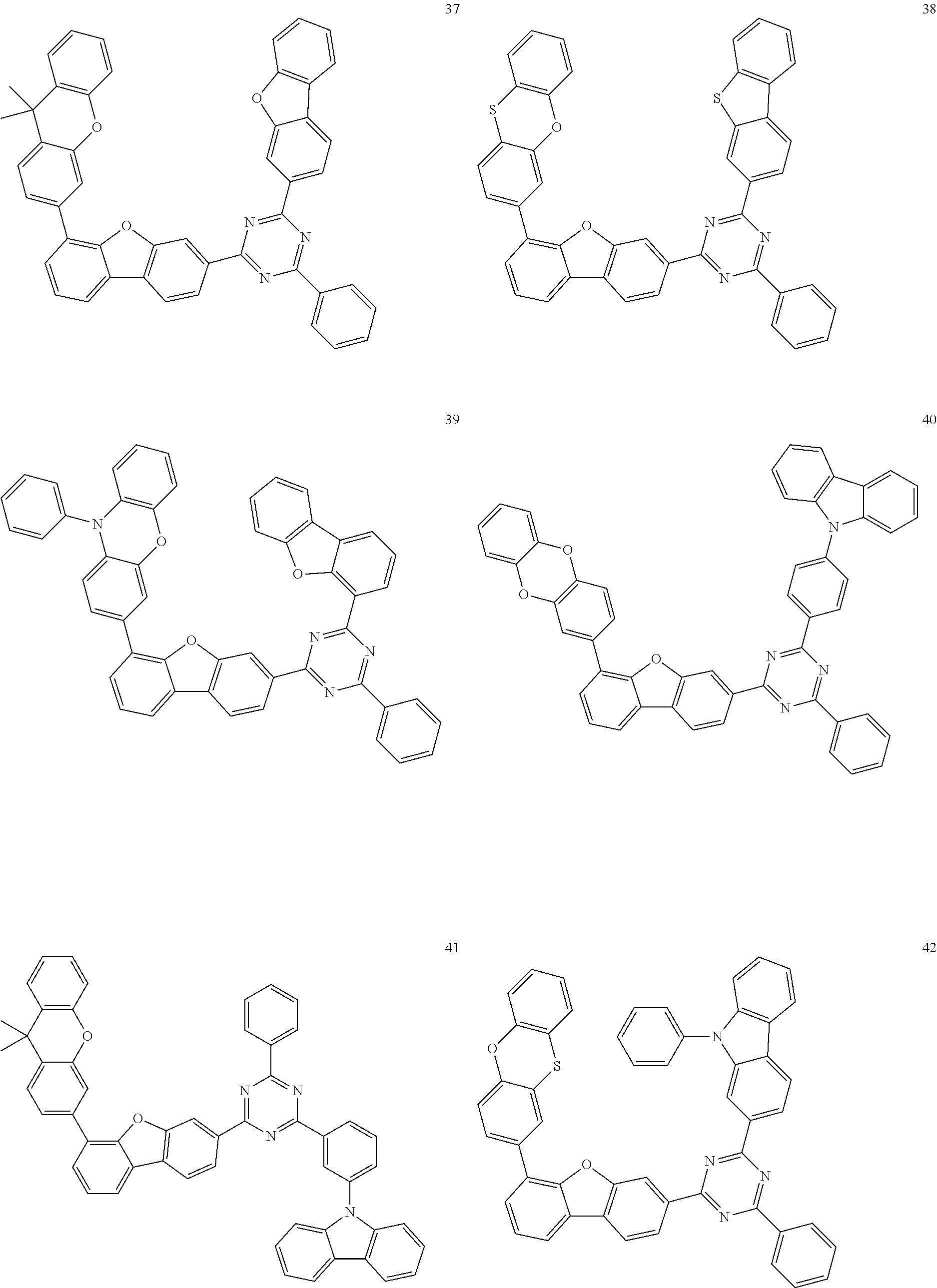

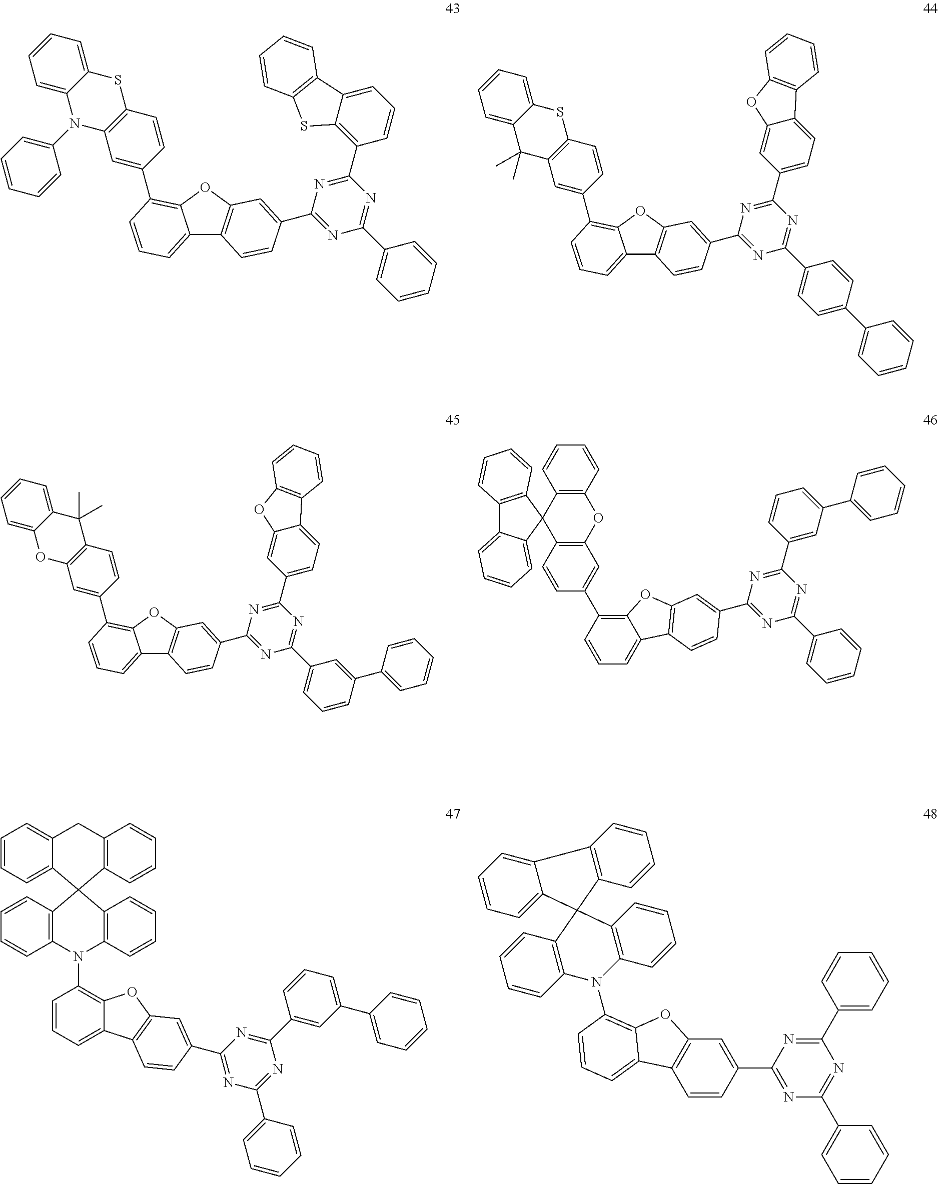

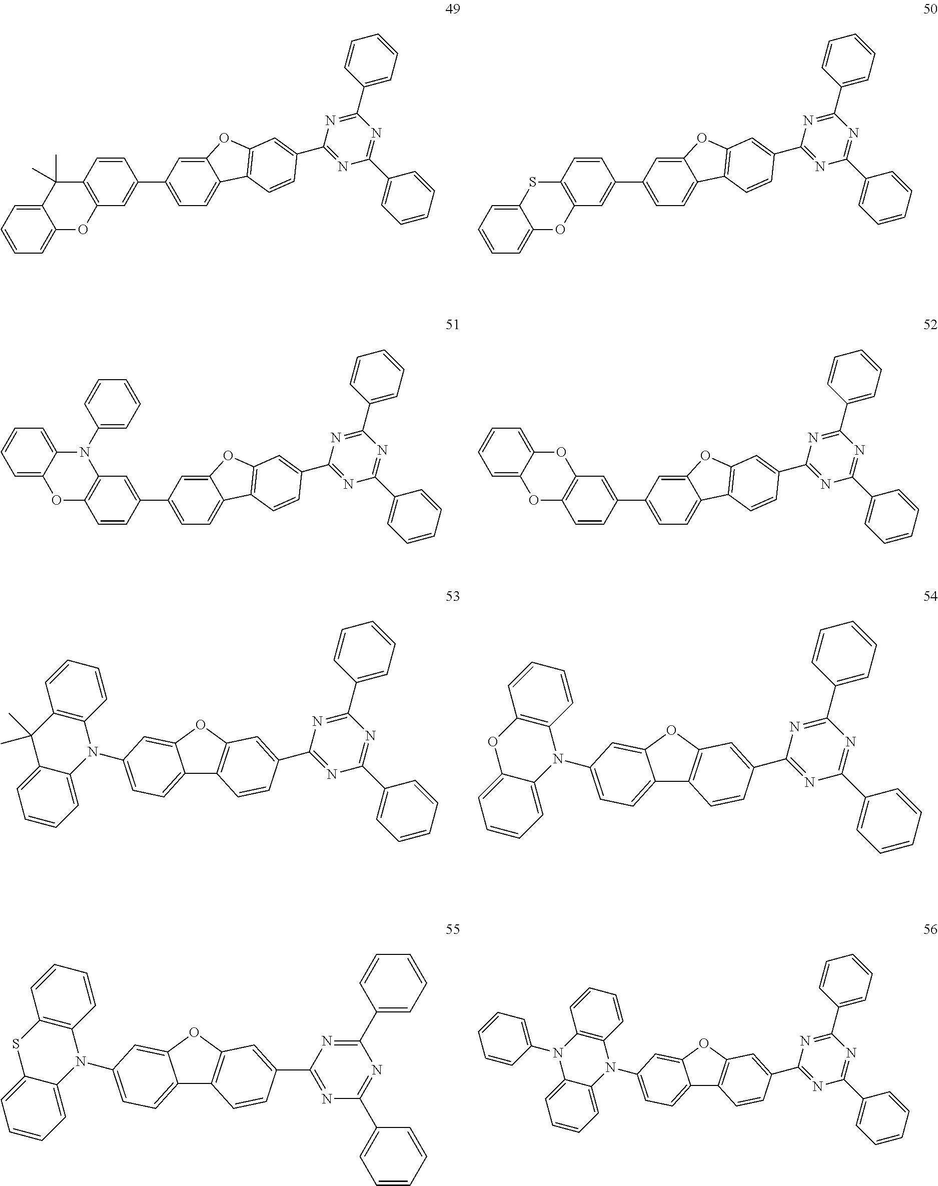

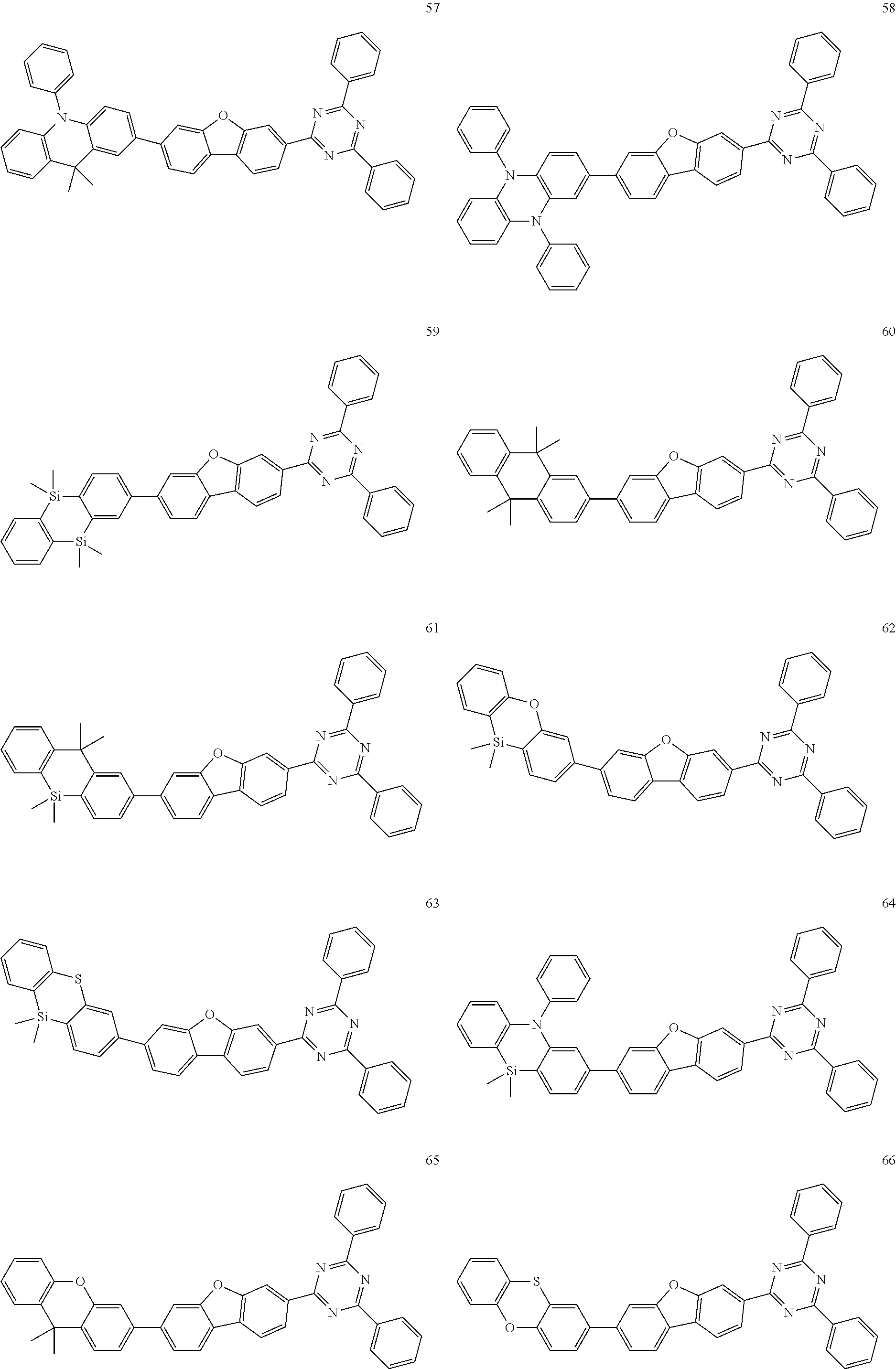

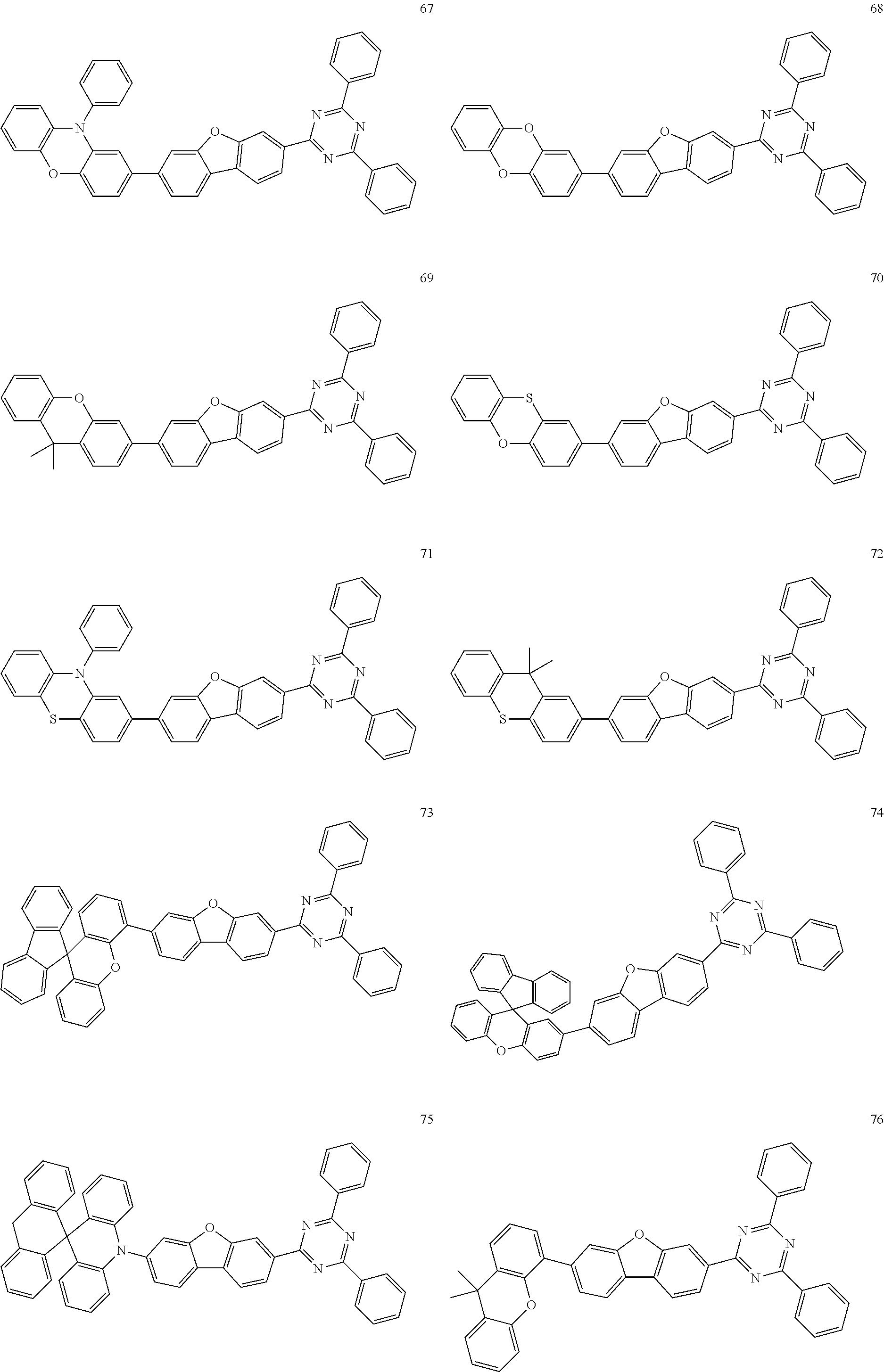

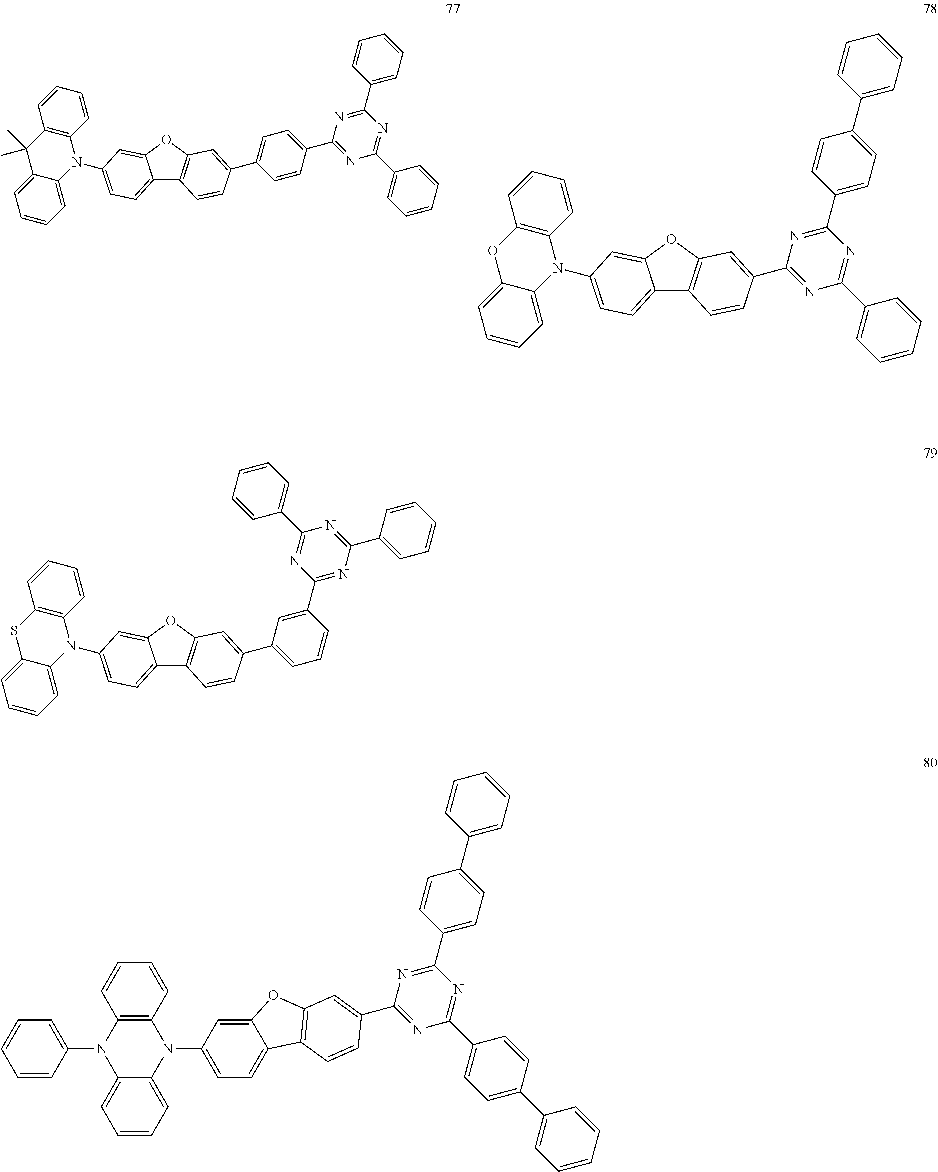 [SMILES: CC1(C)c2ccccc2N(c2ccc3c(c2)oc2cc(-c4ccc(-c5nc(-c6ccccc6)nc(-c6ccccc6)n5)cc4)ccc23)c2ccccc21 ; c1ccc(-c2ccc(-c3nc(-c4ccccc4)nc(-c4ccc5c(c4)oc4cc(N6c7ccccc7Oc7ccccc76)ccc45)n3)cc2)cc1 ; c1ccc(-c2nc(-c3ccccc3)nc(-c3cccc(-c4ccc5c(c4)oc4cc(N6c7ccccc7Sc7ccccc76)ccc45)c3)n2)cc1 ; c1ccc(-c2ccc(-c3nc(-c4ccc(-c5ccccc5)cc4)nc(-c4ccc5c(c4)oc4cc(N6c7ccccc7N(c7ccccc7)c7ccccc76)ccc45)n3)cc2)cc1]

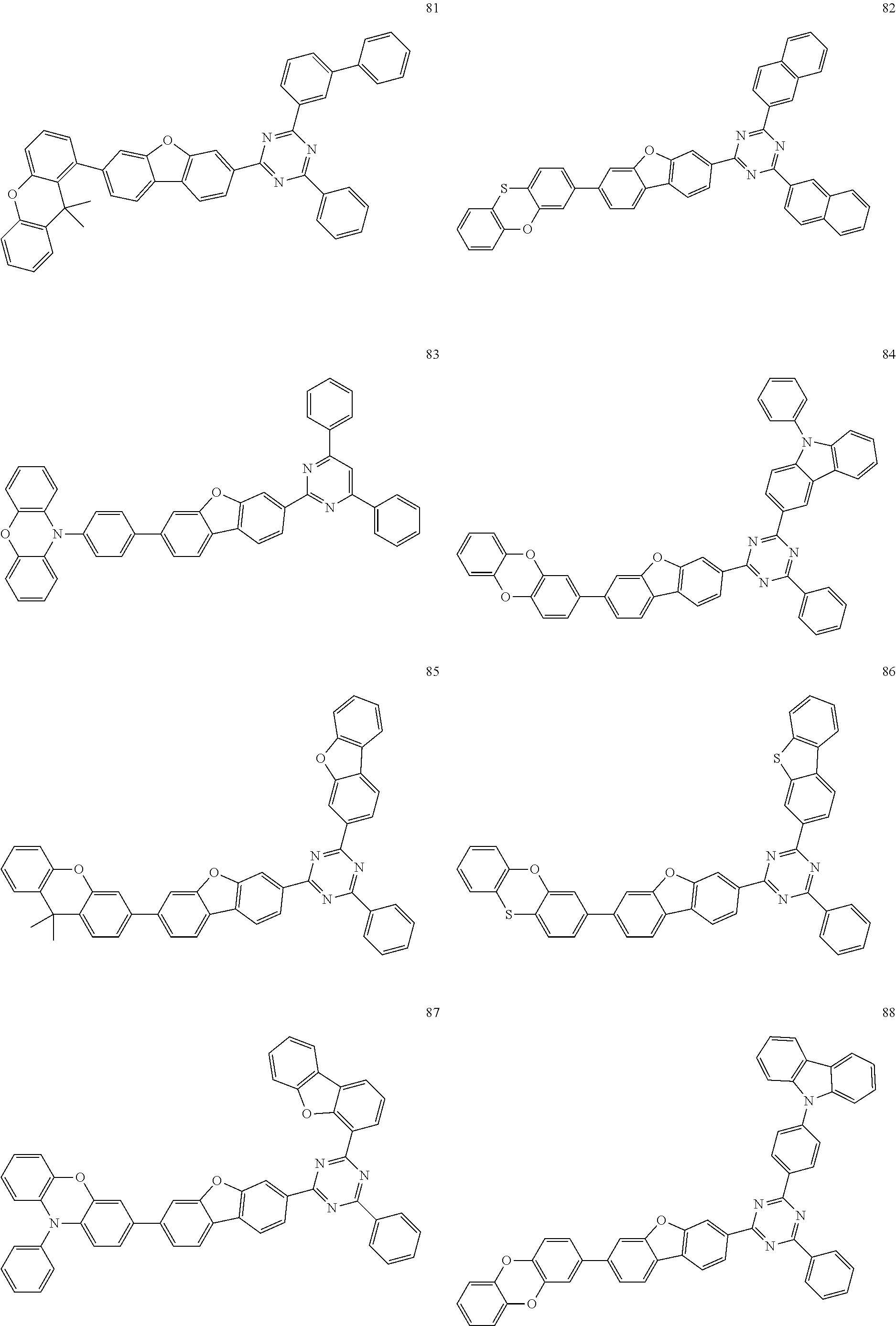

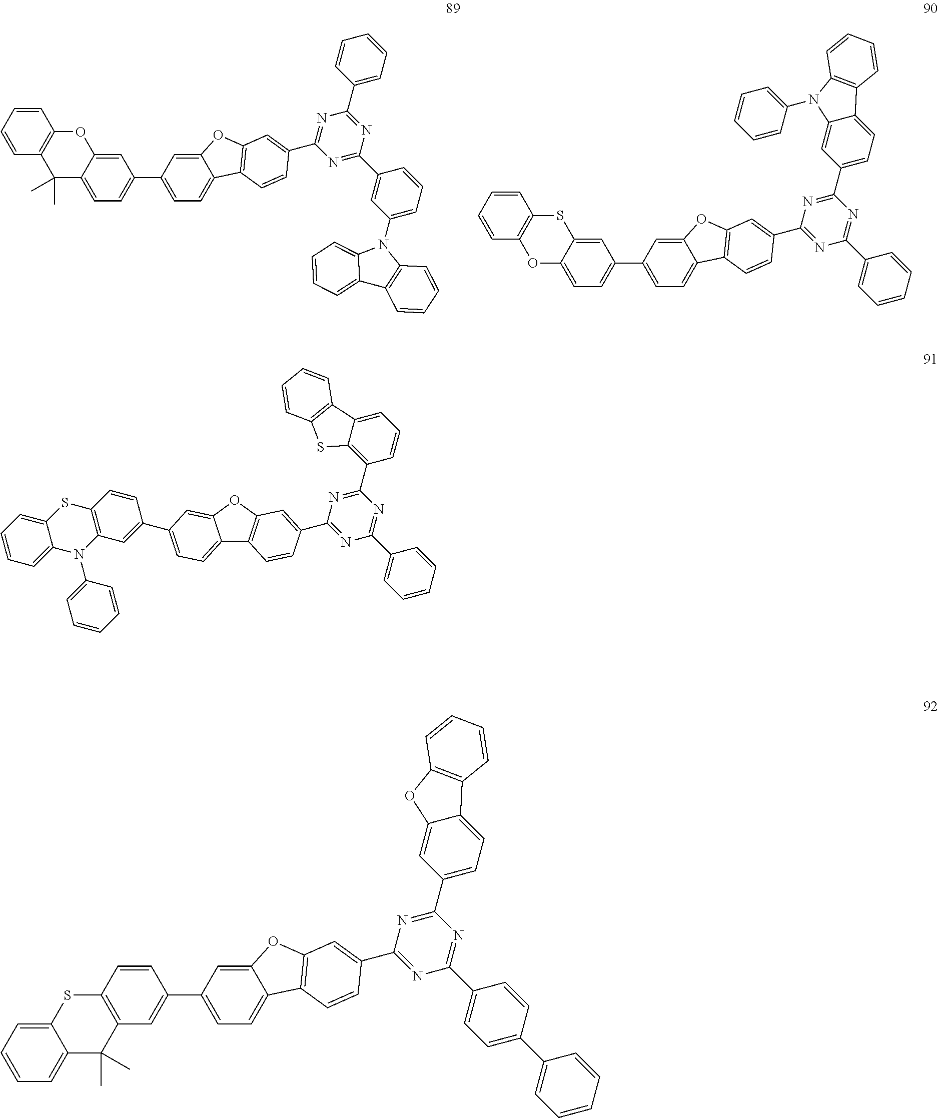 [SMILES: CC1(C)c2ccccc2Oc2cc(-c3ccc4c(c3)oc3cc(-c5nc(-c6ccccc6)nc(-c6cccc(-n7c8ccccc8c8ccccc87)c6)n5)ccc34)ccc21 ; c1ccc(-c2nc(-c3ccc4c(c3)oc3cc(-c5ccc6c(c5)Sc5ccccc5O6)ccc34)nc(-c3ccc4c5ccccc5n(-c5ccccc5)c4c3)n2)cc1 ; c1ccc(-c2nc(-c3ccc4c(c3)oc3cc(-c5ccc6c(c5)N(c5ccccc5)c5ccccc5S6)ccc34)nc(-c3cccc4c3sc3ccccc34)n2)cc1 ; CC1(C)c2ccccc2Sc2ccc(-c3ccc4c(c3)oc3cc(-c5nc(-c6ccc(-c7ccccc7)cc6)nc(-c6ccc7c(c6)oc6ccccc67)n5)ccc34)cc21]

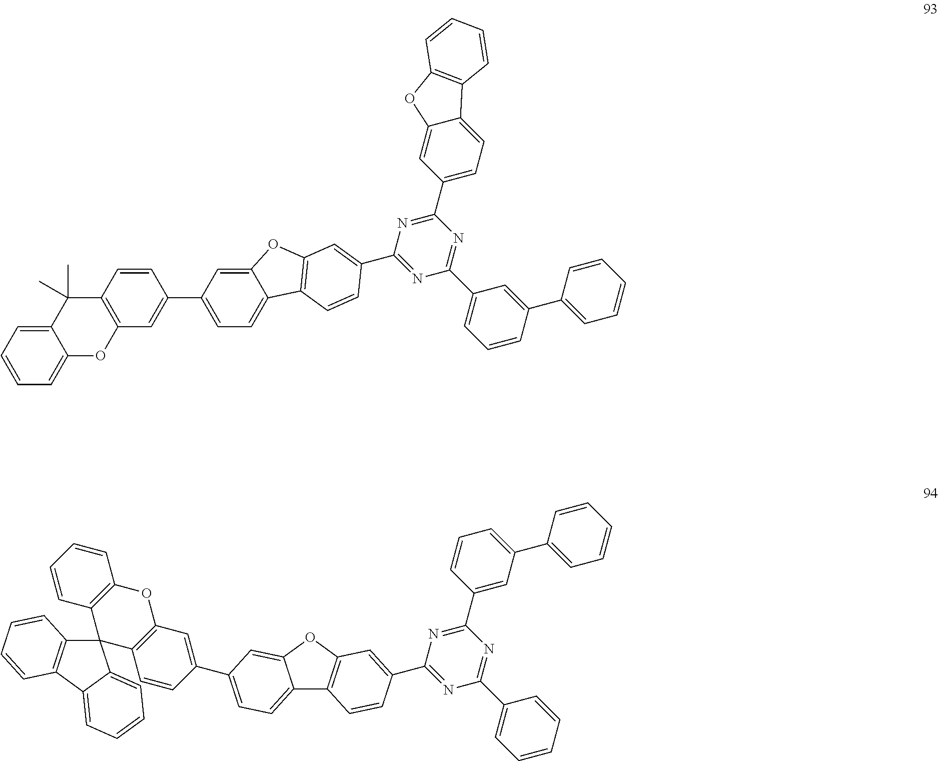 [SMILES: CC1(C)c2ccccc2Oc2cc(-c3ccc4c(c3)oc3cc(-c5nc(-c6cccc(-c7ccccc7)c6)nc(-c6ccc7c(c6)oc6ccccc67)n5)ccc34)ccc21 ; c1ccc(-c2cccc(-c3nc(-c4ccccc4)nc(-c4ccc5c(c4)oc4cc(-c6ccc7c(c6)Oc6ccccc6C76c7ccccc7-c7ccccc76)ccc45)n3)c2)cc1]

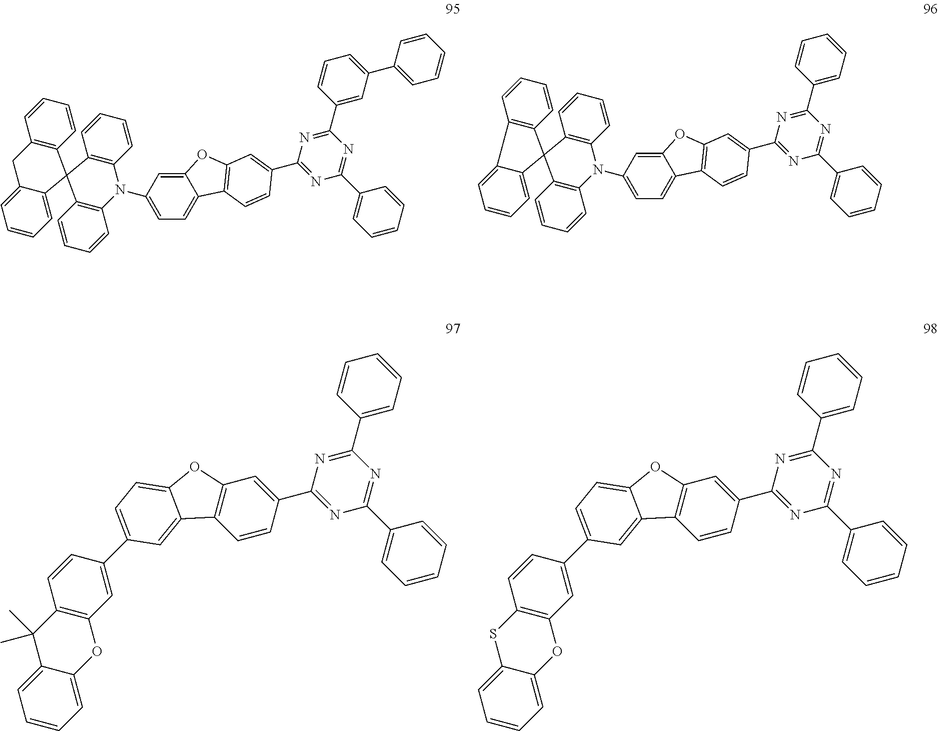 [SMILES: c1ccc(-c2cccc(-c3nc(-c4ccccc4)nc(-c4ccc5c(c4)oc4cc(N6c7ccccc7C7(c8ccccc8Cc8ccccc87)c7ccccc76)ccc45)n3)c2)cc1 ; c1ccc(-c2nc(-c3ccccc3)nc(-c3ccc4c(c3)oc3cc(N5c6ccccc6C6(c7ccccc7-c7ccccc76)c6ccccc65)ccc34)n2)cc1 ; CC1(C)c2ccccc2Oc2cc(-c3ccc4oc5cc(-c6nc(-c7ccccc7)nc(-c7ccccc7)n6)ccc5c4c3)ccc21 ; c1ccc(-c2nc(-c3ccccc3)nc(-c3ccc4c(c3)oc3ccc(-c5ccc6c(c5)Oc5ccccc5S6)cc34)n2)cc1]

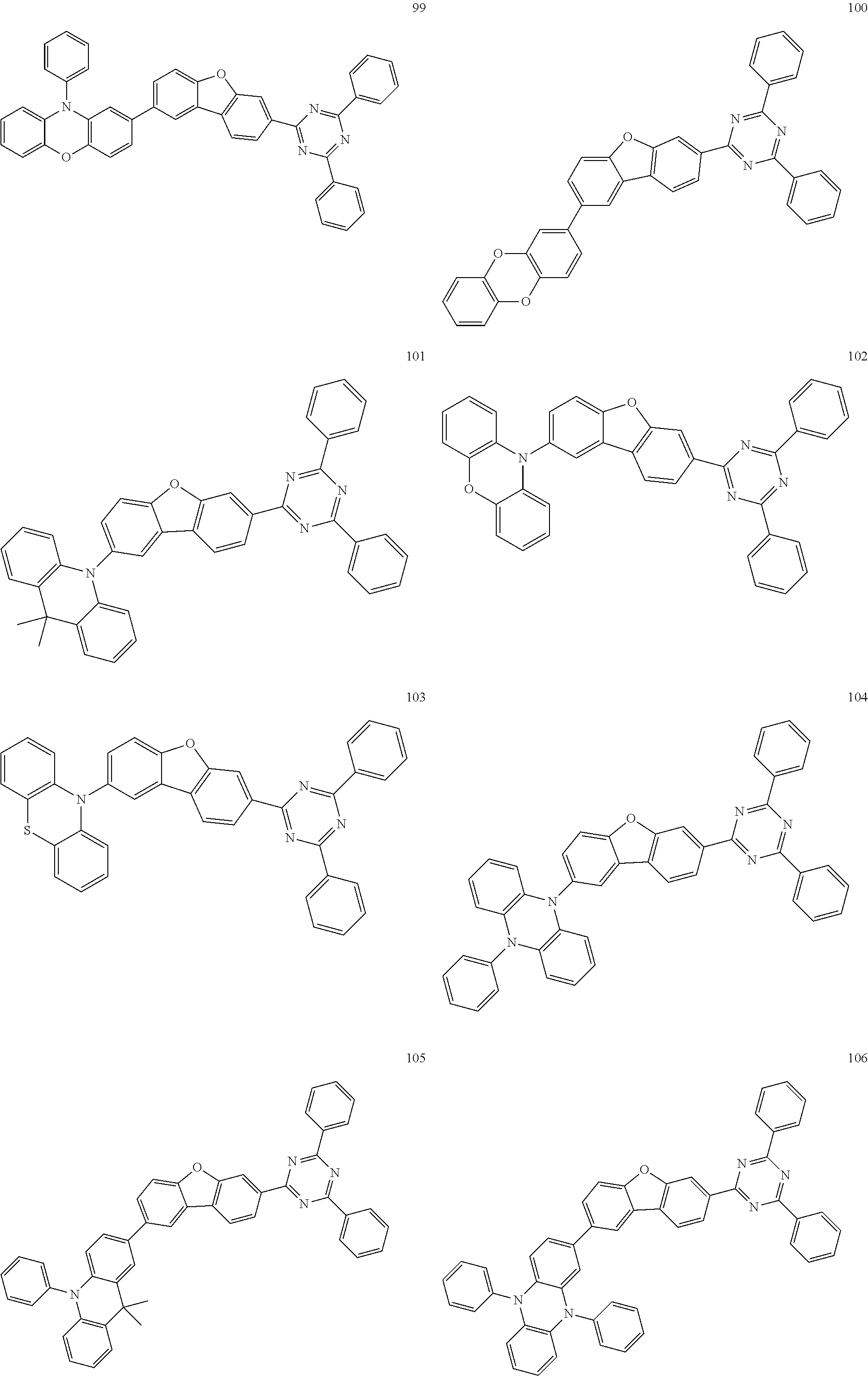

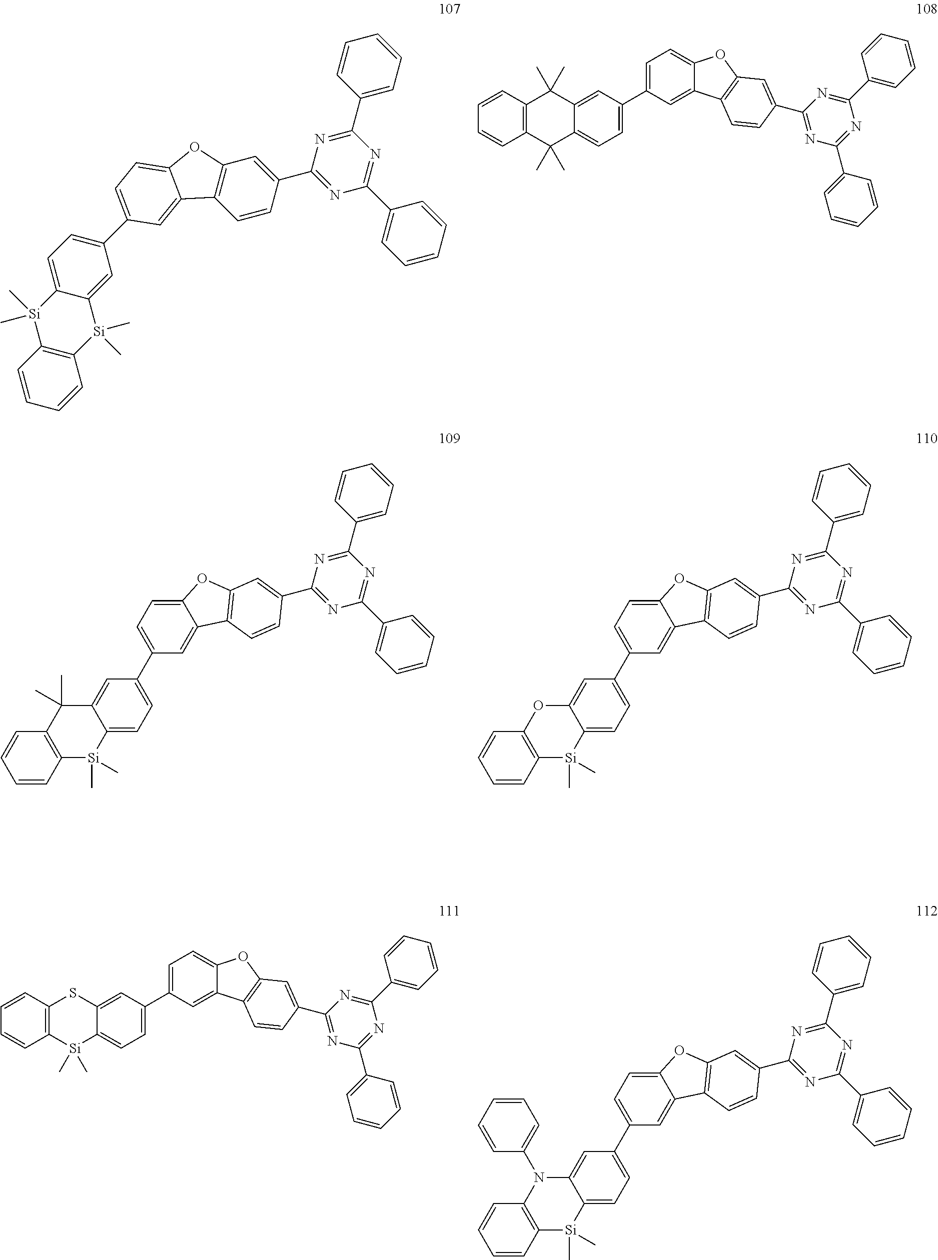

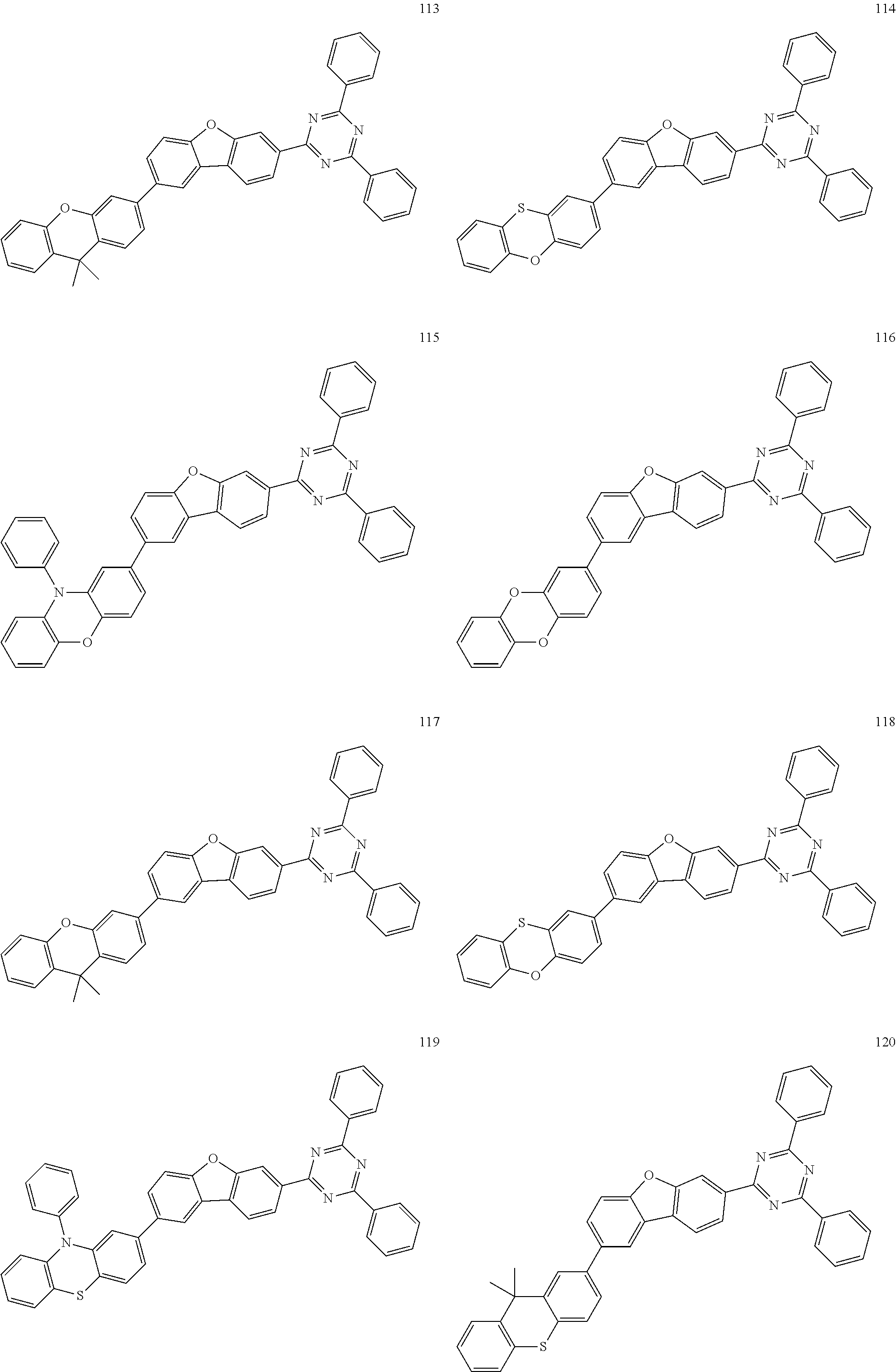

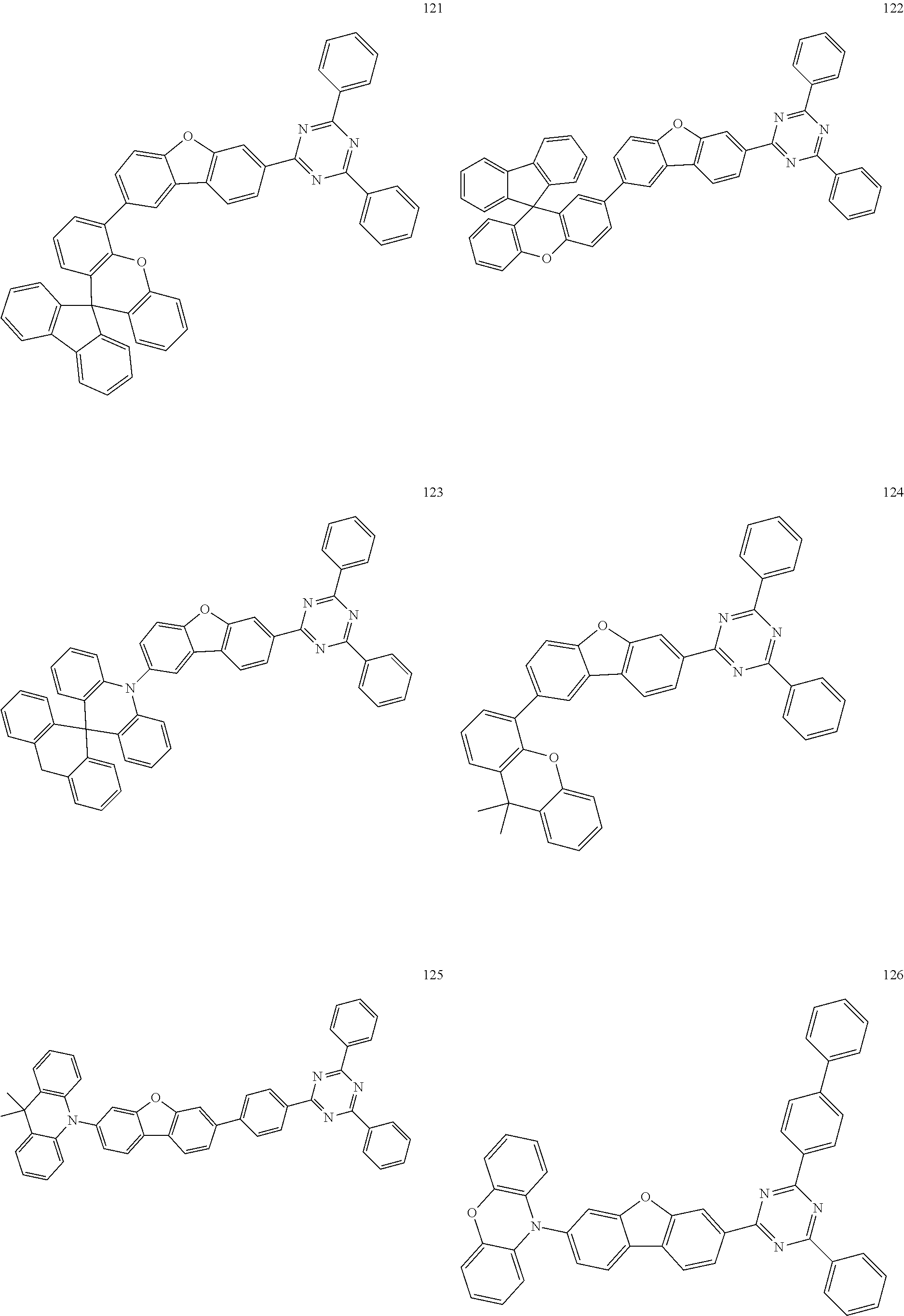

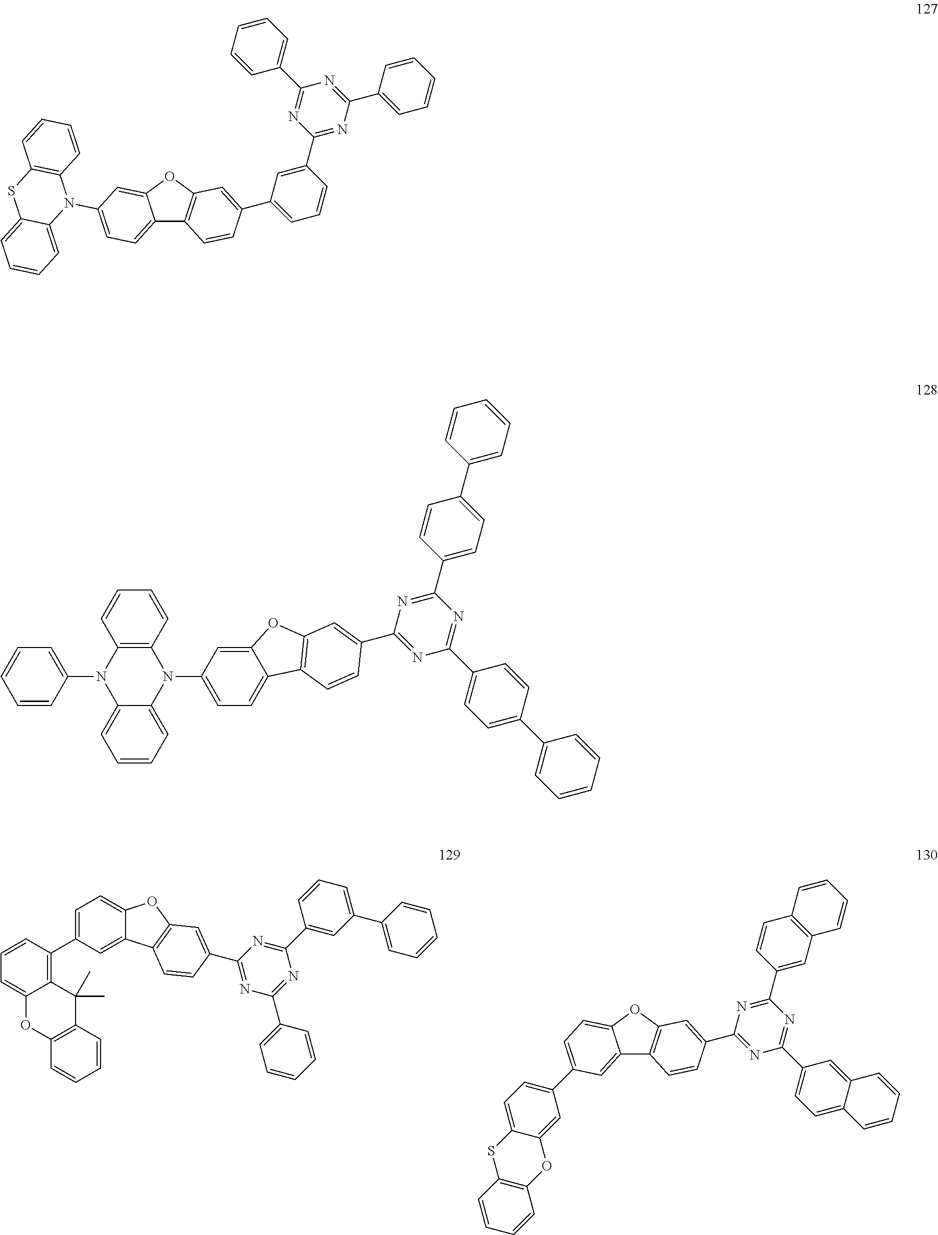 [SMILES: c1ccc(-c2nc(-c3ccccc3)nc(-c3cccc(-c4ccc5c(c4)oc4cc(N6c7ccccc7Sc7ccccc76)ccc45)c3)n2)cc1 ; c1ccc(-c2ccc(-c3nc(-c4ccc(-c5ccccc5)cc4)nc(-c4ccc5c(c4)oc4cc(N6c7ccccc7N(c7ccccc7)c7ccccc76)ccc45)n3)cc2)cc1 ; CC1(C)c2ccccc2Oc2cccc(-c3ccc4oc5cc(-c6nc(-c7ccccc7)nc(-c7cccc(-c8ccccc8)c7)n6)ccc5c4c3)c21 ; c1ccc2c(c1)Oc1cc(-c3ccc4oc5cc(-c6nc(-c7ccc8ccccc8c7)nc(-c7ccc8ccccc8c7)n6)ccc5c4c3)ccc1S2]

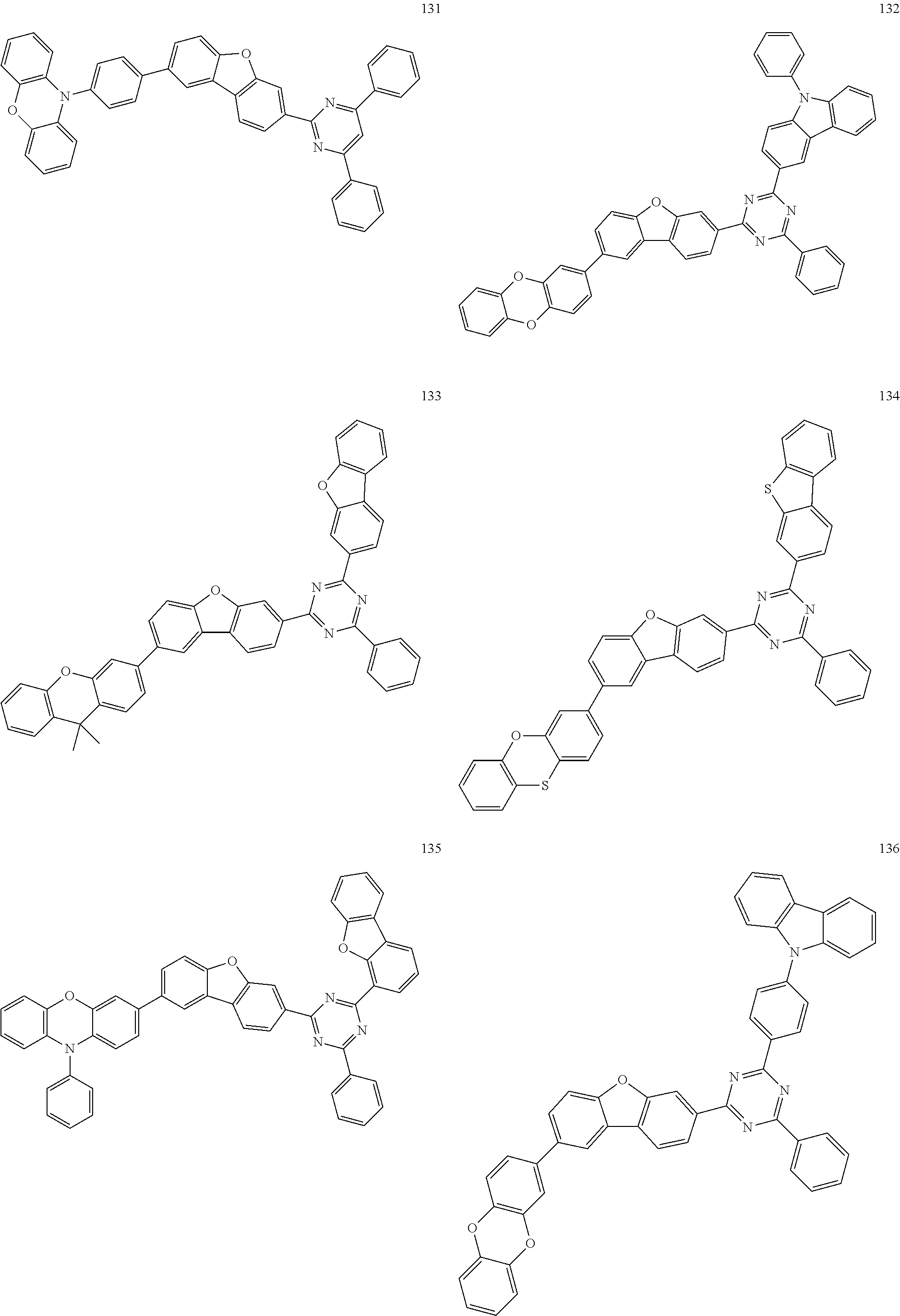

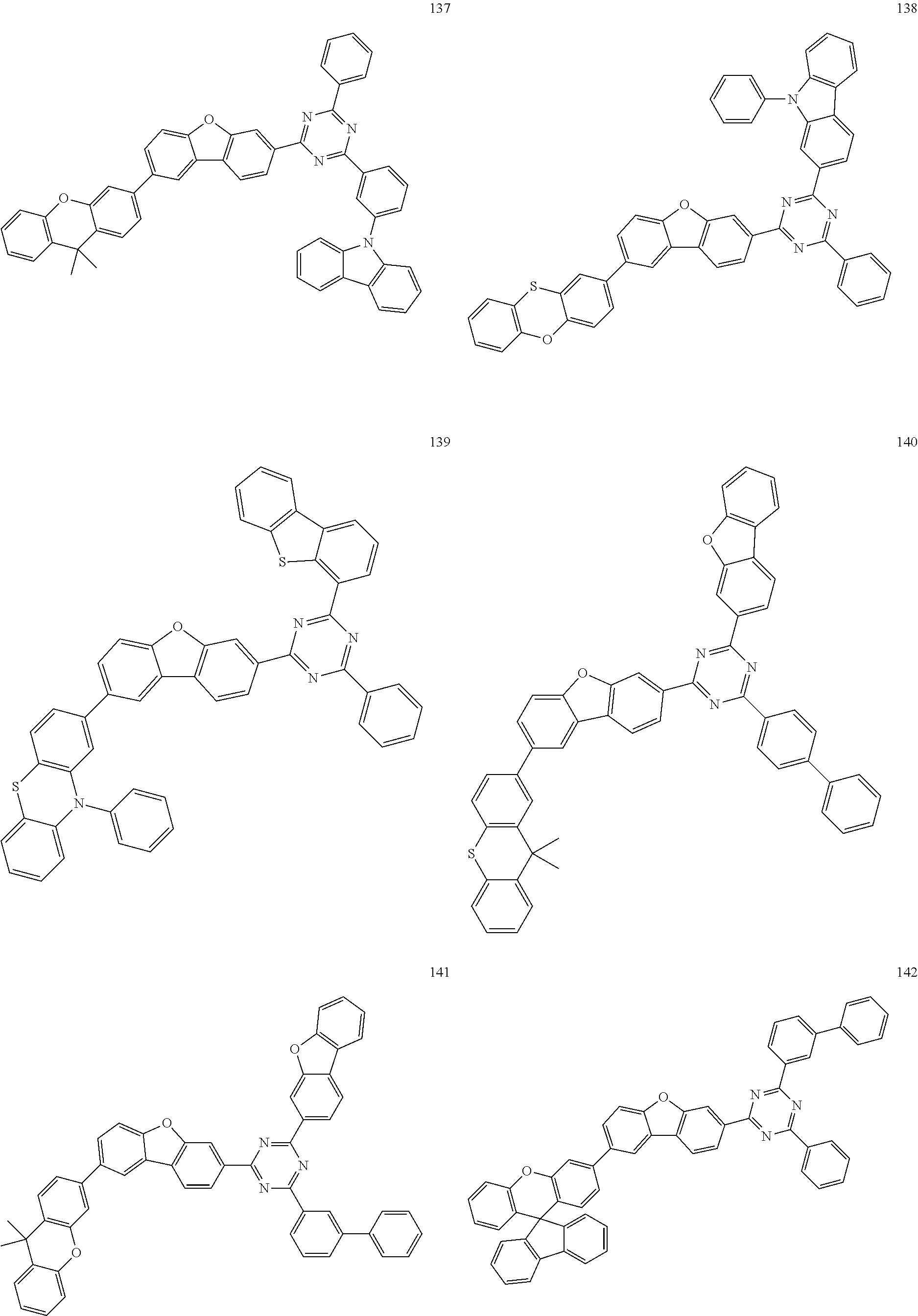

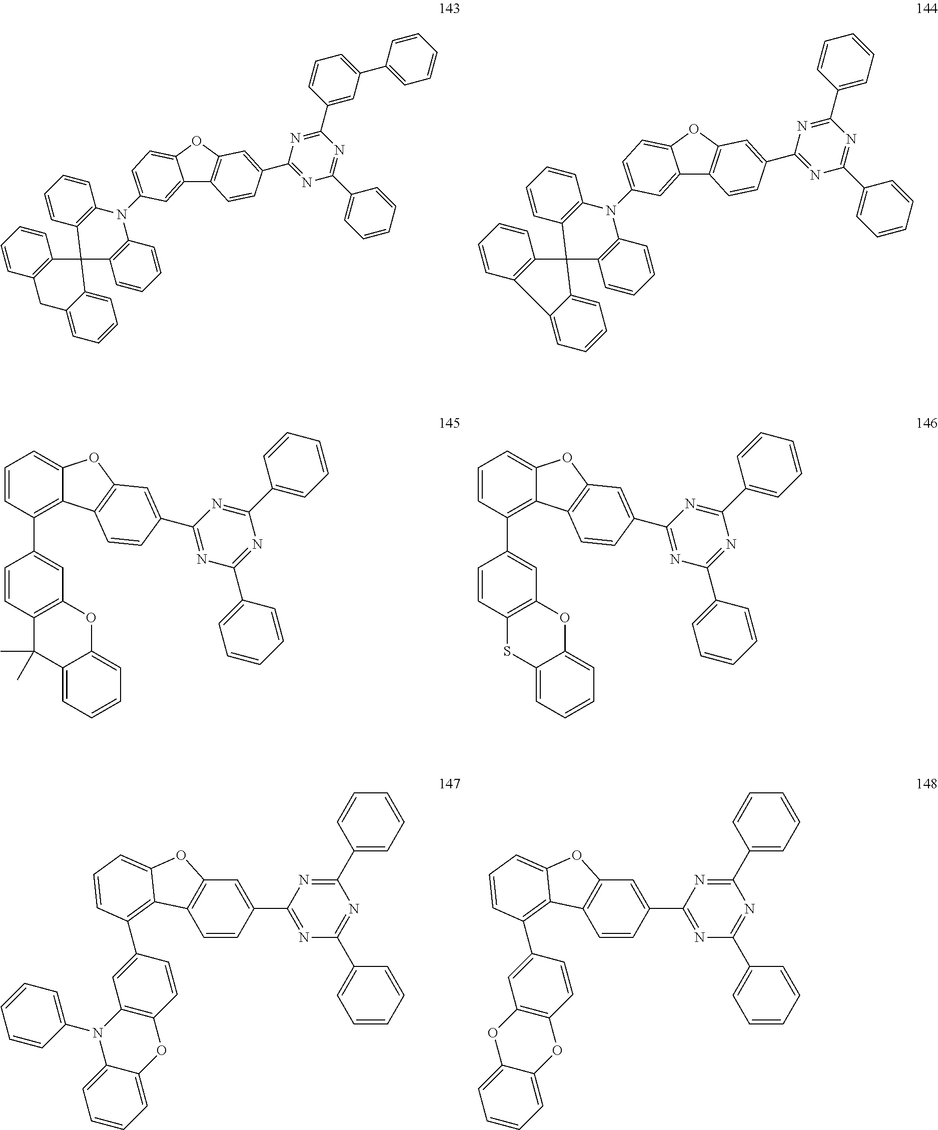

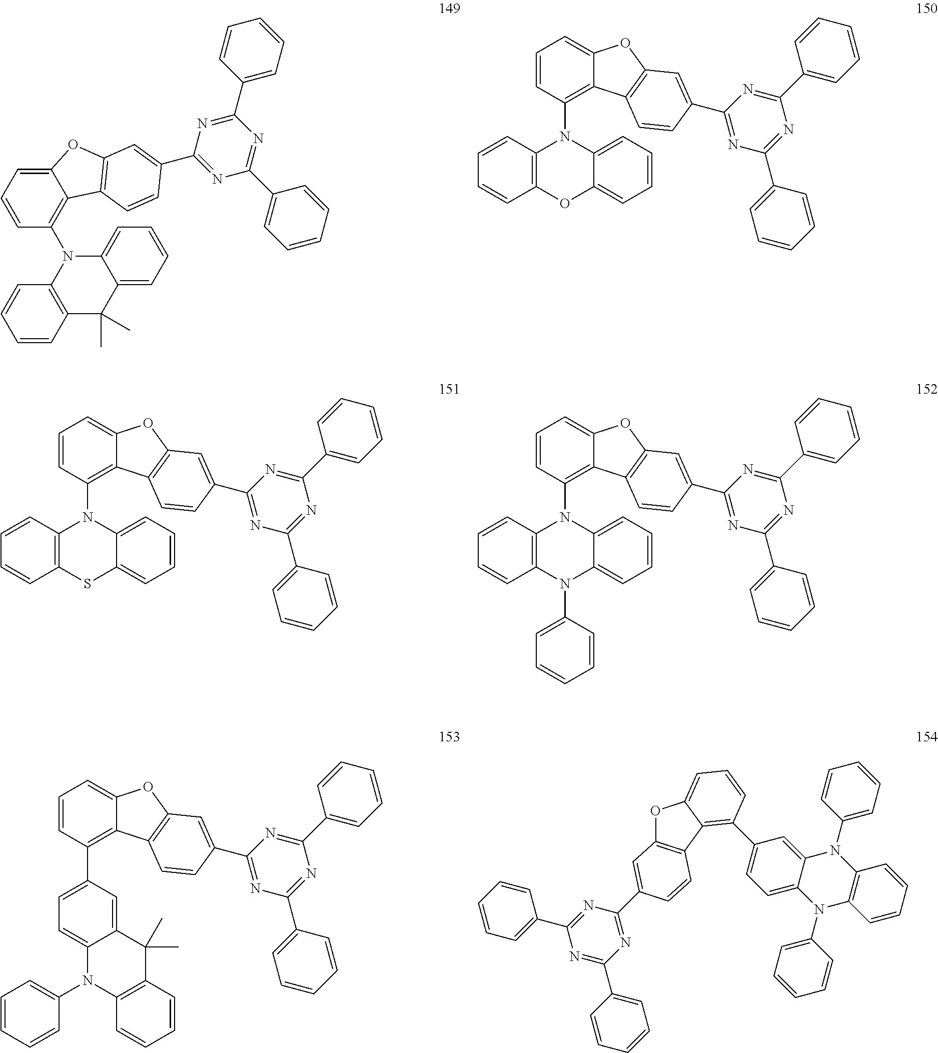

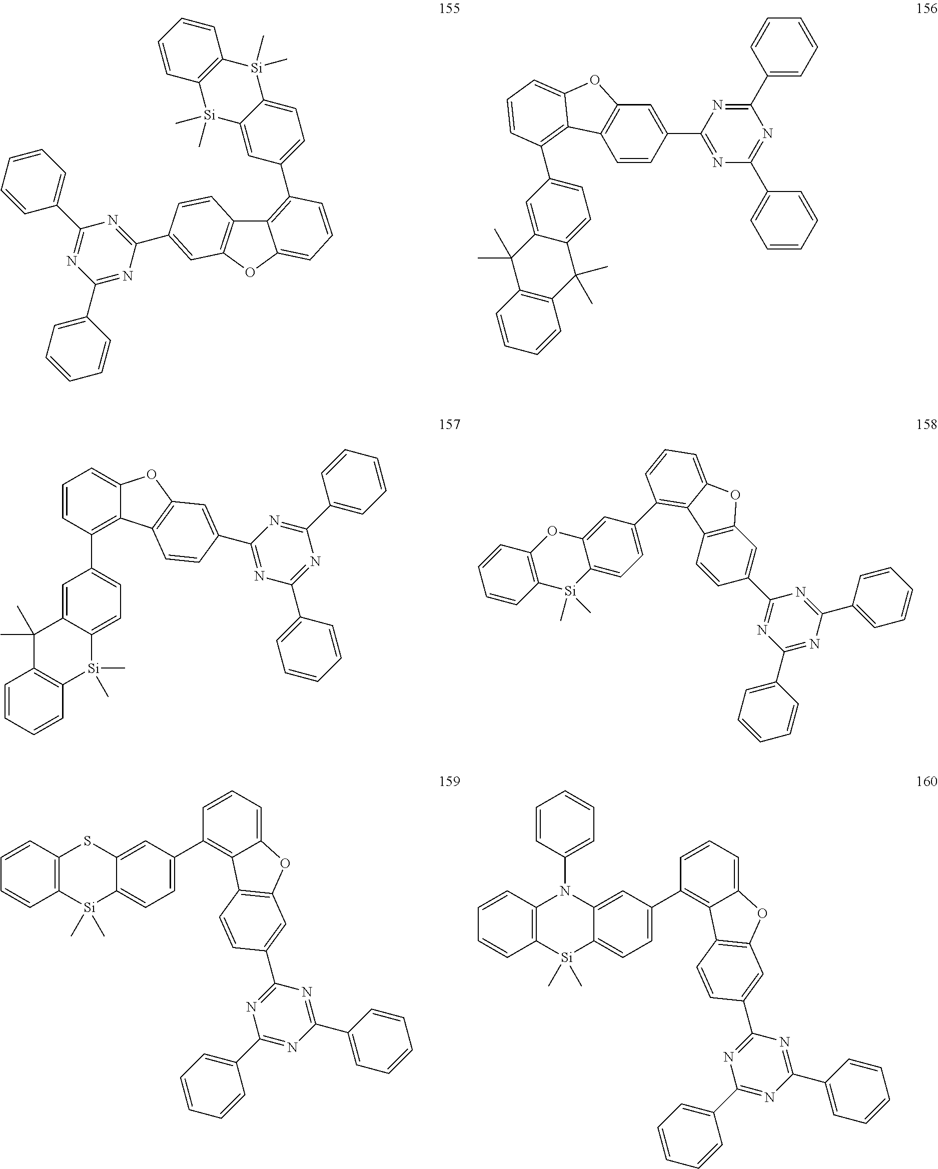

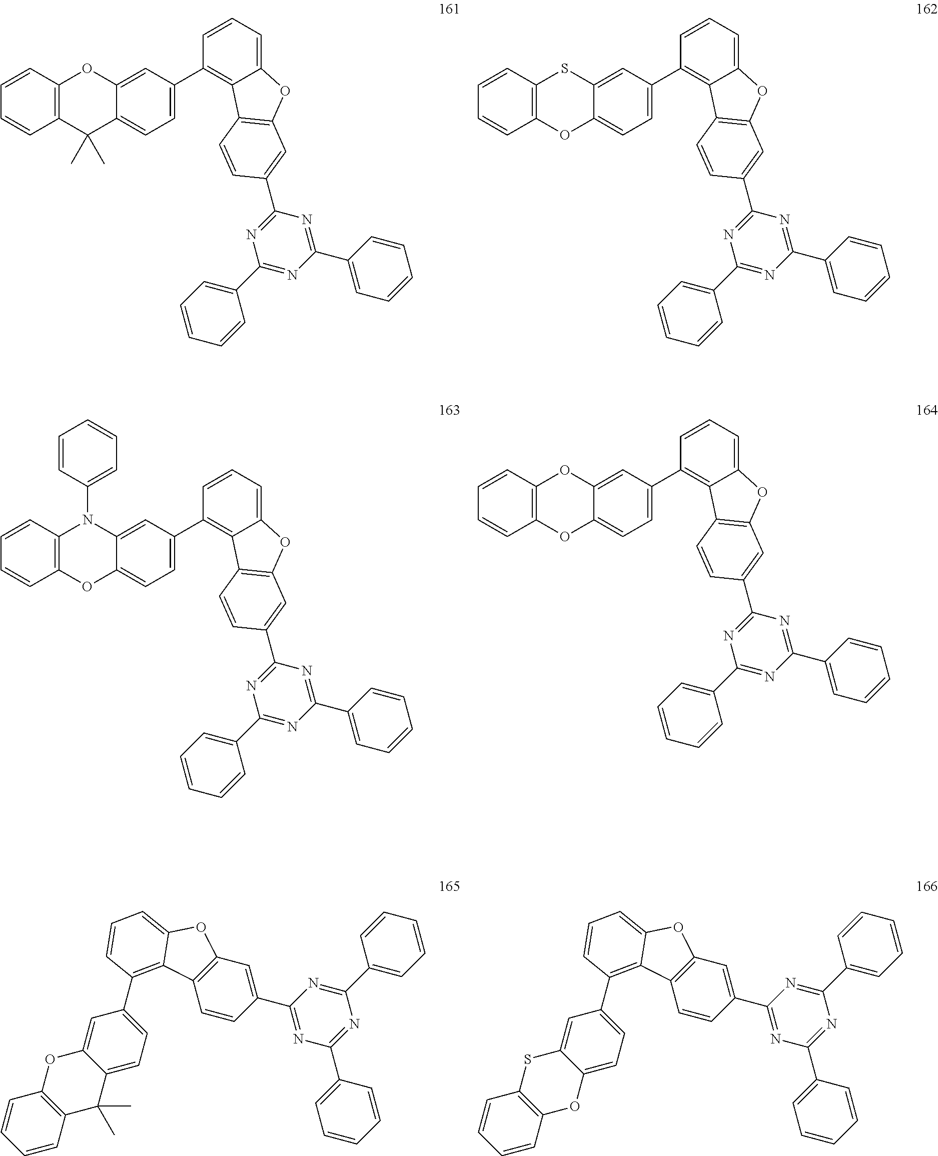 [SMILES: CC1(C)c2ccccc2Oc2cc(-c3cccc4oc5cc(-c6nc(-c7ccccc7)nc(-c7ccccc7)n6)ccc5c34)ccc21 ; c1ccc(-c2nc(-c3ccccc3)nc(-c3ccc4c(c3)oc3cccc(-c5ccc6c(c5)Sc5ccccc5O6)c34)n2)cc1 ; c1ccc(-c2nc(-c3ccccc3)nc(-c3ccc4c(c3)oc3cccc(-c5ccc6c(c5)N(c5ccccc5)c5ccccc5O6)c34)n2)cc1 ; c1ccc(-c2nc(-c3ccccc3)nc(-c3ccc4c(c3)oc3cccc(-c5ccc6c(c5)Oc5ccccc5O6)c34)n2)cc1 ; CC1(C)c2ccccc2Oc2cc(-c3cccc4oc5cc(-c6nc(-c7ccccc7)nc(-c7ccccc7)n6)ccc5c34)ccc21 ; c1ccc(-c2nc(-c3ccccc3)nc(-c3ccc4c(c3)oc3cccc(-c5ccc6c(c5)Sc5ccccc5O6)c34)n2)cc1]

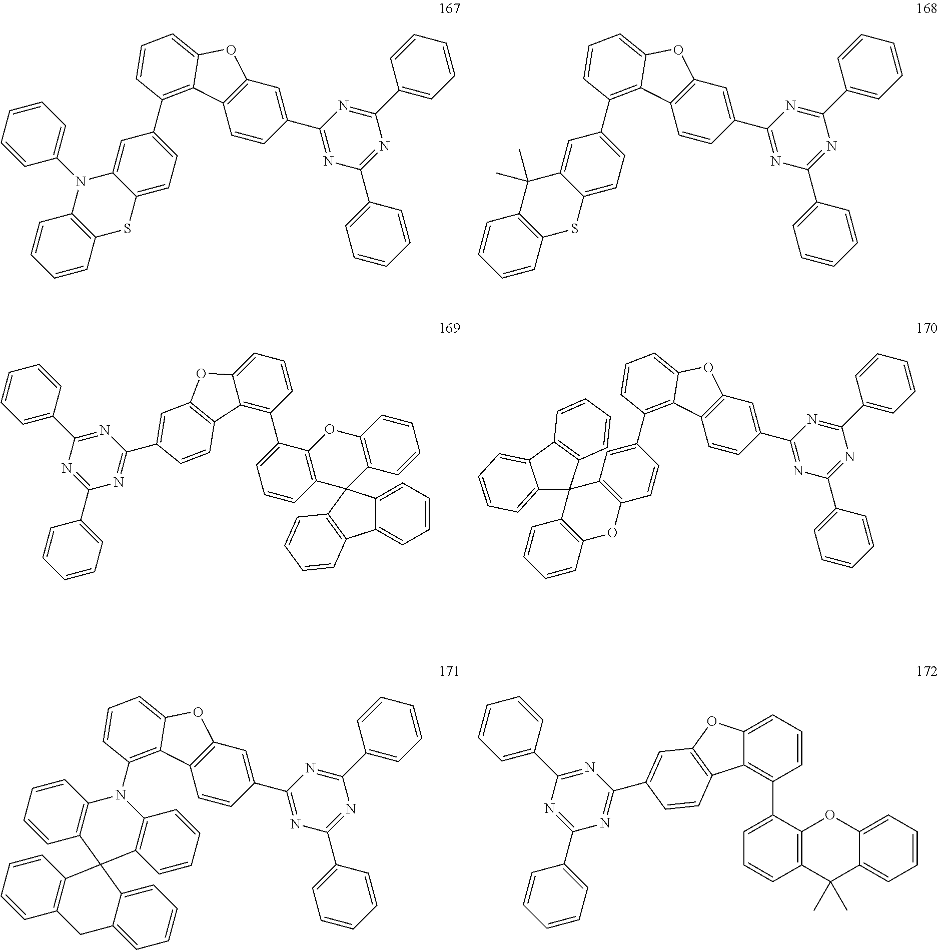

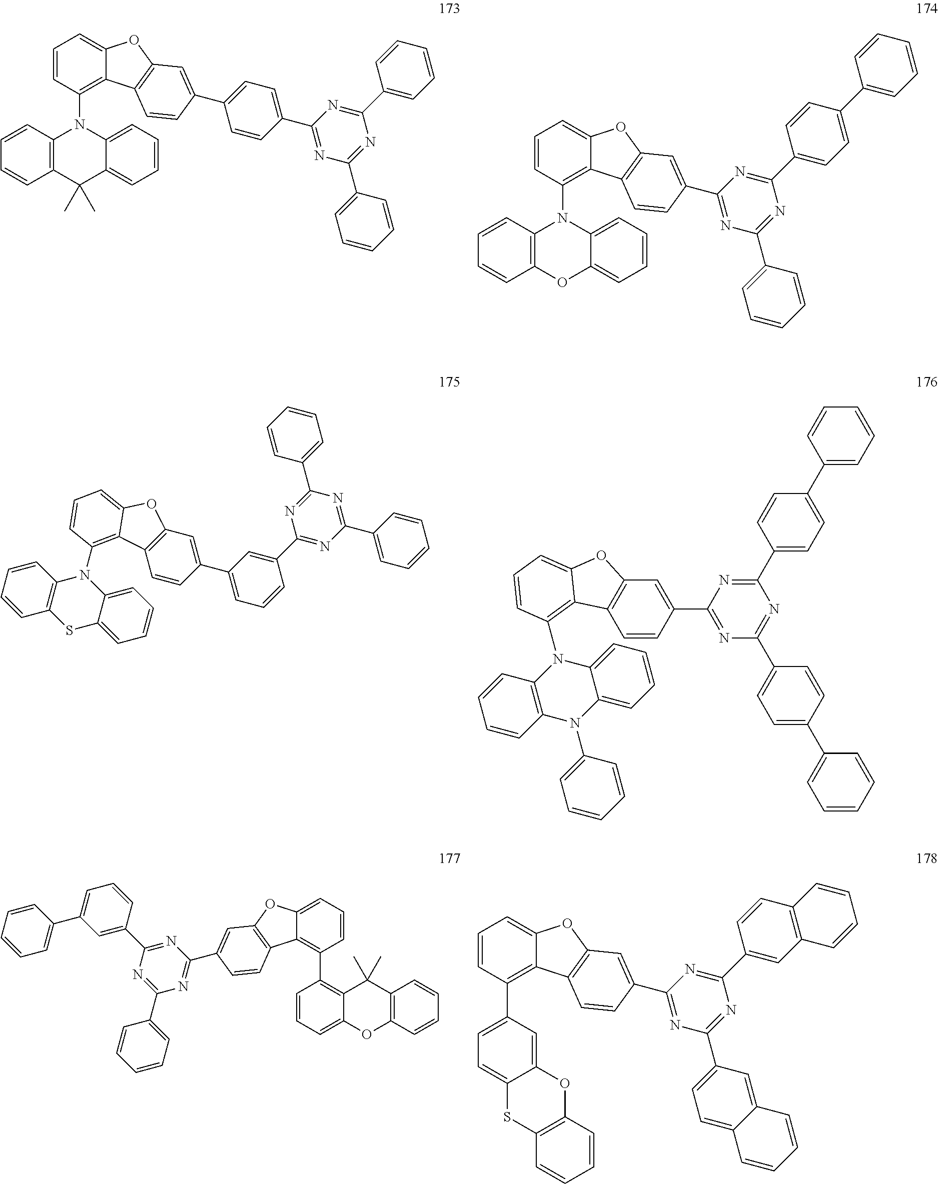

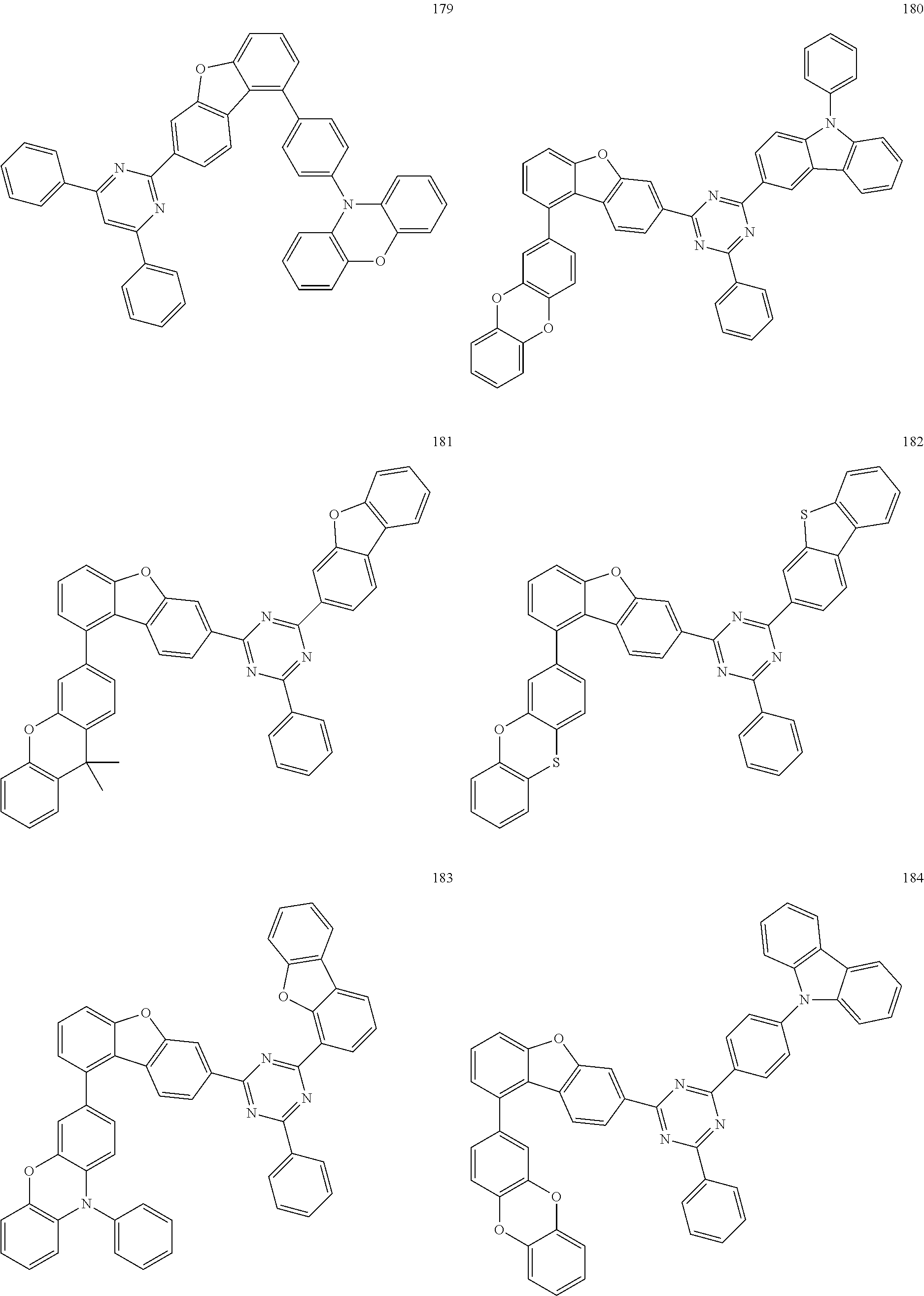

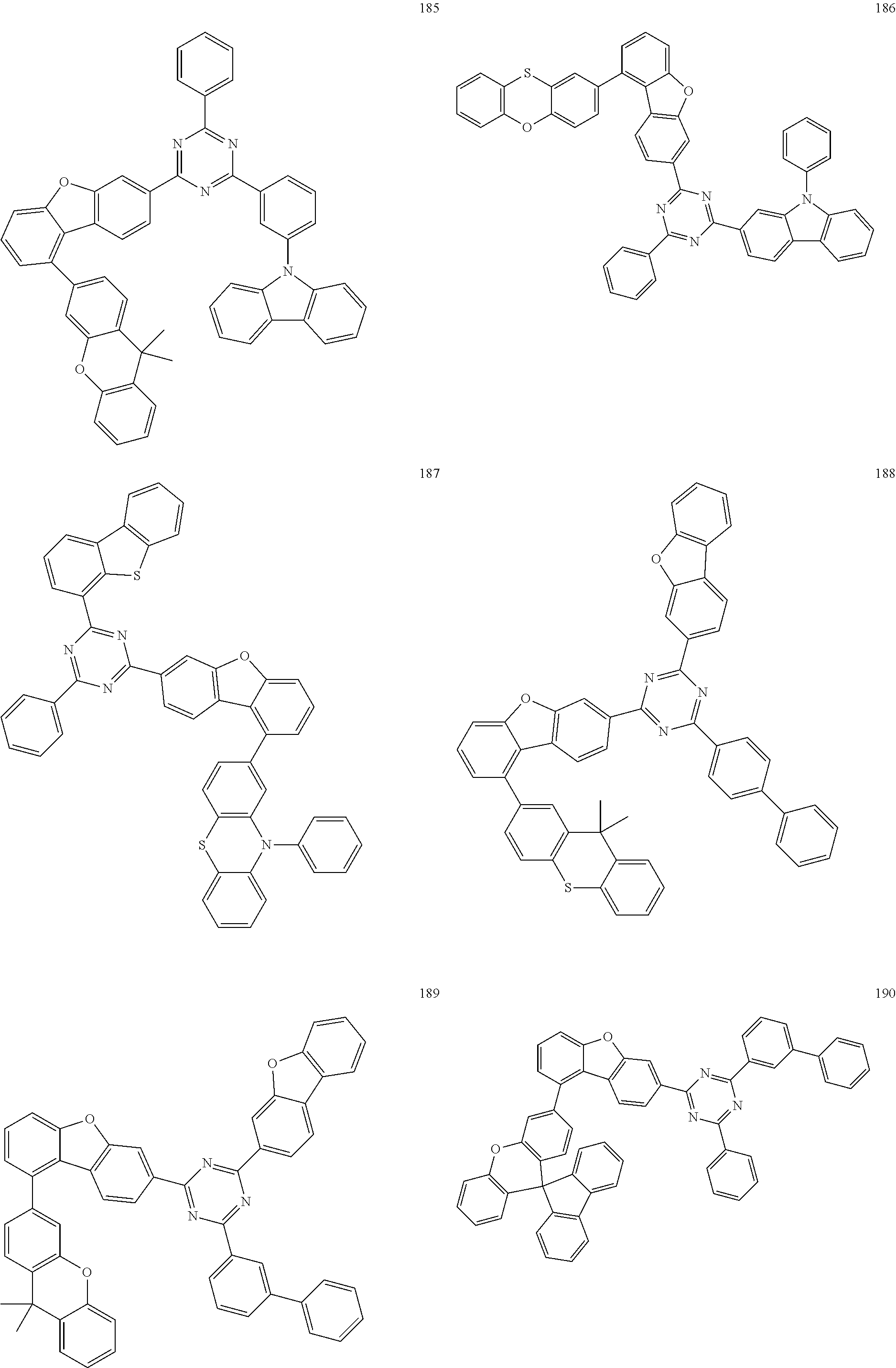

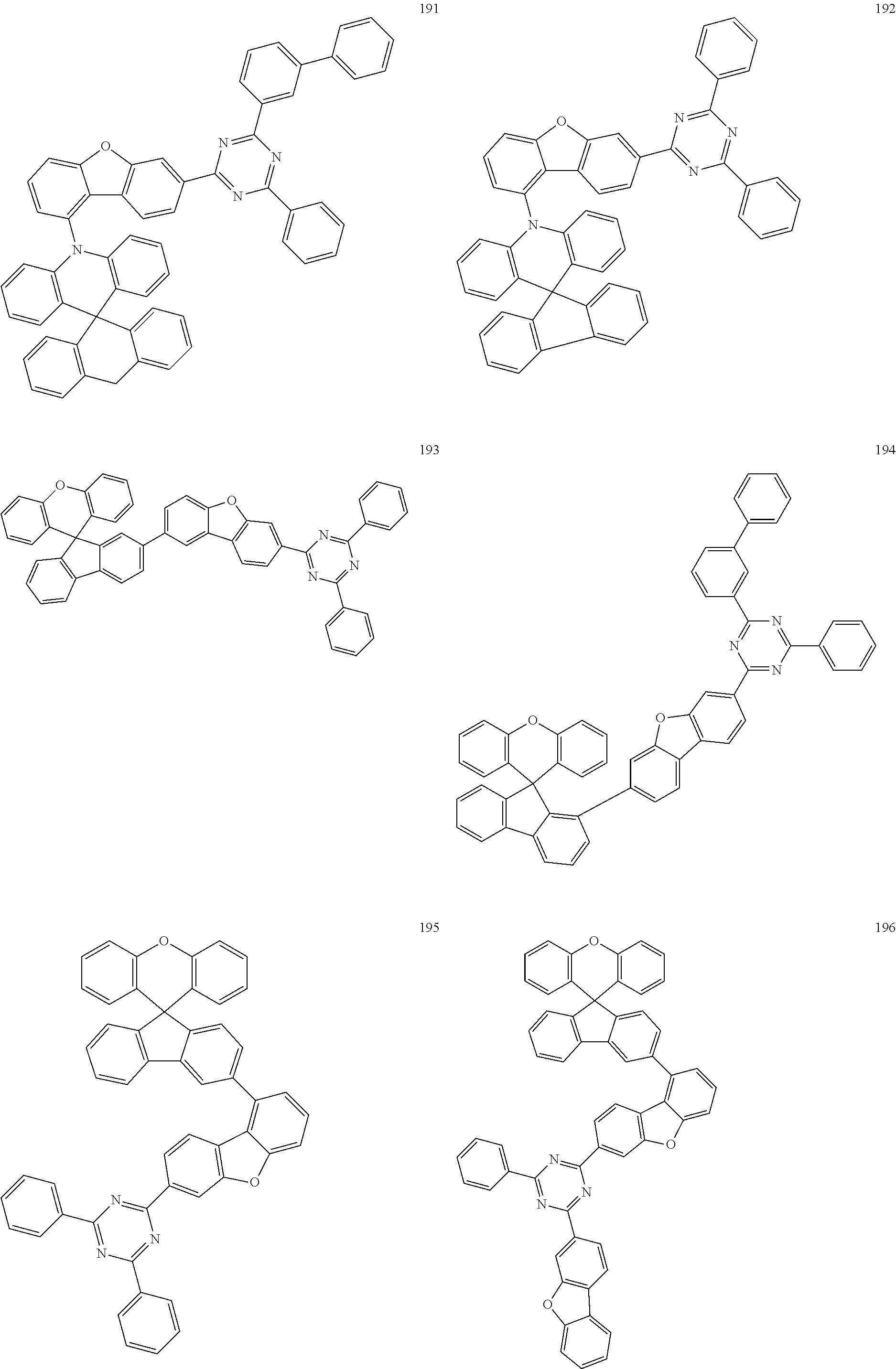

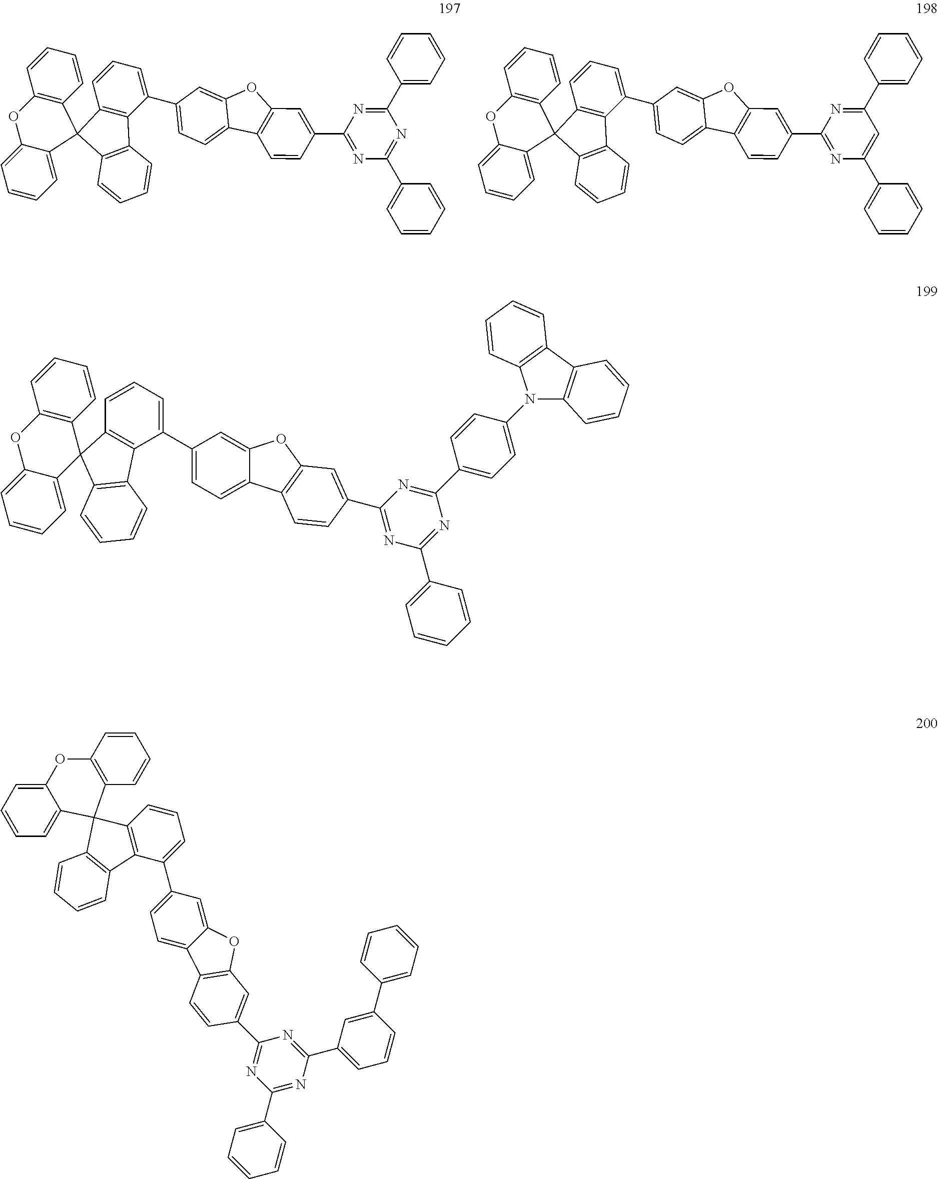 [SMILES: c1ccc(-c2nc(-c3ccccc3)nc(-c3ccc4c(c3)oc3cc(-c5cccc6c5-c5ccccc5C65c6ccccc6Oc6ccccc65)ccc34)n2)cc1 ; c1ccc(-c2cc(-c3ccccc3)nc(-c3ccc4c(c3)oc3cc(-c5cccc6c5-c5ccccc5C65c6ccccc6Oc6ccccc65)ccc34)n2)cc1 ; c1ccc(-c2nc(-c3ccc(-n4c5ccccc5c5ccccc54)cc3)nc(-c3ccc4c(c3)oc3cc(-c5cccc6c5-c5ccccc5C65c6ccccc6Oc6ccccc65)ccc34)n2)cc1 ; c1ccc(-c2cccc(-c3nc(-c4ccccc4)nc(-c4ccc5c(c4)oc4cc(-c6cccc7c6-c6ccccc6C76c7ccccc7Oc7ccccc76)ccc45)n3)c2)cc1]

In addition, by introducing various substituents to the structure of Chemical Formula 1, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

In addition, one embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound according to Chemical Formula 1.

Specific descriptions on the heterocyclic compound represented by Chemical Formula 1 are the same as the descriptions provided above.

In one embodiment of the present application, the first electrode may be an anode, and the second electrode may be a cathode.

In another embodiment, the first electrode may be a cathode, and the second electrode may be an anode.

In one embodiment of the present application, the organic light emitting device may be a blue organic light emitting device, and the heterocyclic compound according to Chemical Formula 1 may be used as a material of the blue organic light emitting device. For example, the heterocyclic compound according to Chemical Formula 1 may be comprised in a host material of a blue light emitting layer of the blue organic light emitting device.

In one embodiment of the present application, the organic light emitting device may be a green organic light emitting device, and the heterocyclic compound according to Chemical Formula 1 may be used as a material of the green organic light emitting device. For example, the heterocyclic compound according to Chemical Formula 1 may be included in a host material of a green light emitting layer of the green organic light emitting device.

In one embodiment of the present application, the organic light emitting device may be a red organic light emitting device, and the heterocyclic compound according to Chemical Formula 1 may be used as a material of the red organic light emitting device. For example, the heterocyclic compound according to Chemical Formula 1 may be included in a host material of a red light emitting layer of the red organic light emitting device.

The organic light emitting device of the present disclosure may be manufactured using common organic light emitting device manufacturing methods and materials except that one or more of the organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may be formed in a multilayer structure in which two or more organic material layers are laminated. For example, the organic light emitting device of the present disclosure may have a structure comprising a hole injection layer, a hole transfer layer, a light emitting layer, an electron transfer layer, an electron injection layer and the like as the organic material layer. However, the structure of the organic light emitting device is not limited thereto, and may comprise a smaller number of organic material layers.

In the organic light emitting device of the present disclosure, the organic material layer may comprise a light emitting layer, and the light emitting layer may comprise the heterocyclic compound.

In another organic light emitting device, the organic material layer comprises a light emitting layer, the light emitting layer comprises a host material, and the host material may comprise the heterocyclic compound.

As another example, the organic material layer comprising the heterocyclic compound comprises the heterocyclic compound represented by Chemical Formula 1 as a host, and an iridium-based dopant may be used therewith.

In the organic light emitting device of the present disclosure, the organic material layer comprises an electron injection layer or an electron transfer layer, and the electron transfer layer or the electron injection layer may comprise the heterocyclic compound.

In another organic light emitting device, the organic material layer comprises an electron blocking layer or a hole blocking layer, and the electron blocking layer or the hole blocking layer may comprise the heterocyclic compound.

The organic light emitting device of the present disclosure may further comprise one, two or more layers selected from the group consisting of a light emitting layer, a hole injection layer, a hole transfer layer, an electron injection layer, an electron transfer layer, an electron blocking layer and a hole blocking layer.

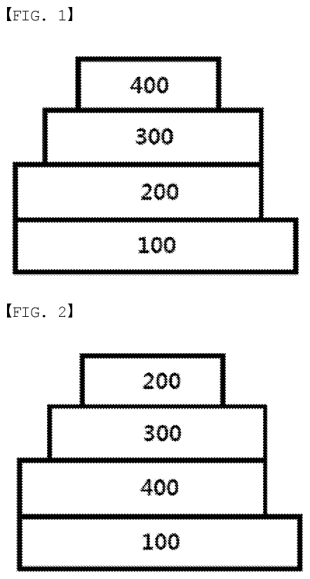

FIG. 1 to FIG. 3 illustrate a lamination order of electrodes and organic material layers of an organic light emitting device according to one embodiment of the present application. However, the scope of the present application is not limited to these diagrams, and structures of organic light emitting devices known in the art may also be used in the present application.

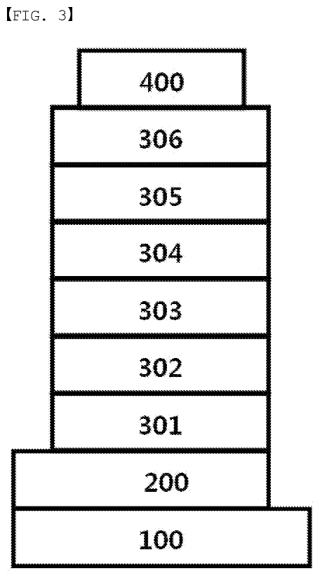

FIG. 1 illustrates an organic light emitting device in which an anode (200), an organic material layer (300) and a cathode (400) are consecutively laminated on a substrate (100) However, the structure is not limited to such a structure, and as illustrated in FIG. 2, an organic light emitting device in which a cathode, an organic material layer and an anode are consecutively laminated on a substrate may also be obtained.

FIG. 3 illustrates a case of the organic material layer being a multilayer. The organic light emitting device according to FIG. 3 comprises a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, layers other than the light emitting layer may not be included, and other necessary functional layers may be further added.

The organic material layer comprising the compound of Chemical Formula 1 may further comprise other materials as necessary.

In the organic light emitting device according to one embodiment of the present application, materials other than the compound of Chemical Formula 1 are illustrated below, however, these are for illustrative purposes only and not for limiting the scope of the present application, and may be replaced by materials known in the art.

As the anode material, materials having relatively large work function may be used, and transparent conductive oxides, metals, conductive polymers or the like may be used. Specific examples of the anode material comprise metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the cathode material, materials having relatively small work function may be used, and metals, metal oxides, conductive polymers or the like may be used. Specific examples of the cathode material comprise metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrene-sulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection material, LiF is typically used in the art, however, the present application is not limited thereto.

As the light emitting material, red, green or blue light emitting materials may be used, and as necessary, two or more light emitting materials may be mixed and used. Herein, two or more light emitting materials may be used by being deposited as individual sources of supply or by being premixed and deposited as one source of supply. In addition, fluorescent materials may also be used as the light emitting material, however, phosphorescent materials may also be used. As the light emitting material, materials emitting light by bonding electrons and holes injected from an anode and a cathode, respectively, may be used alone, however, materials having a host material and a dopant material involving in light emission together may also be used.

When mixing light emitting material hosts, same series hosts may be mixed, or different series hosts may be mixed. For example, any two or more types of materials among n-type host materials or p-type host materials may be selected and used as a host material of a light emitting layer.

The organic light emitting device according to one embodiment of the present application may be a top-emission type, a bottom-emission type or a dual-emission type depending on the materials used.

The heterocyclic compound according to one embodiment of the present application may also be used in an organic electronic device comprising an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

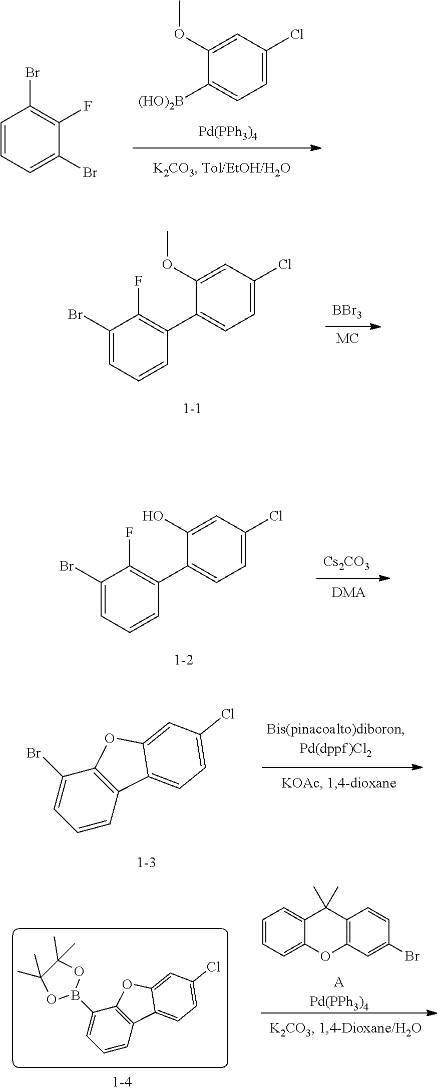

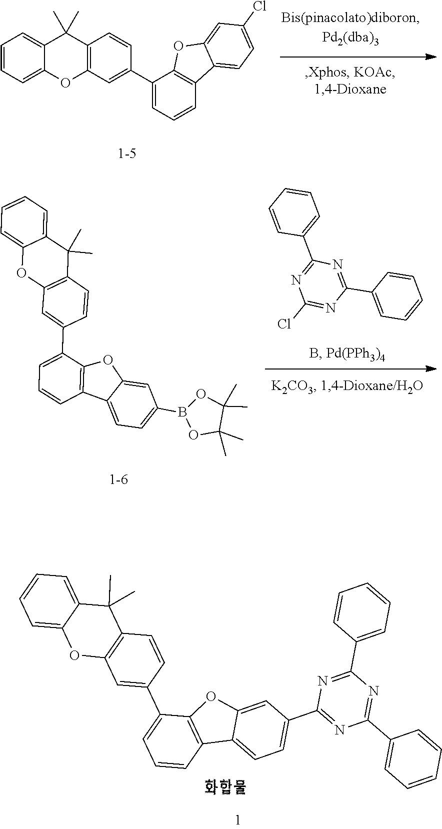 [SMILES: CC1(C)c2ccccc2Oc2cc(-c3cccc4c3oc3cc(Cl)ccc34)ccc21 ; CC1(C)c2ccccc2Oc2cc(-c3cccc4c3oc3cc(B5OC(C)(C)C(C)(C)O5)ccc34)ccc21 ; Clc1nc(-c2ccccc2)nc(-c2ccccc2)n1 ; CC1(C)c2ccccc2Oc2cc(-c3cccc4c3oc3cc(-c5nc(-c6ccccc6)nc(-c6ccccc6)n5)ccc34)ccc21]

Preparation of Compound 1-1

In a one-neck round bottom flask (one neck r.b.f), a mixture of 1,3-dibromo-2-fluorobenzene (50 g, 197 mmol), (4-chloro-2-methoxyphenyl)boronic acid (44 g, 236.4 mmol), tetrakis(triphenylphosphine)palladium(0) (22.7 g, 19.7 mmol), potassium carbonate (5.4 g, 39.4 mol) and toluene/ethanol/water (800 ml/160 ml/160 ml) was refluxed at 110° C. The result was extracted with dichloromethane and dried with MgSO₄. The result was silica gel filtered and then concentrated to obtain Compound 1-1 (63 g, 98%).

Preparation of Compound 1-2

In a one-neck round bottom flask (one neck r.b.f), a mixture of 3-bromo-4′-chloro-2-fluoro-2′-methoxy-1,1′-biphenyl (63 g, 200 mmol) and methylene chloride (MC) (1000 ml) was cooled to a temperature of 0° C., BBr₃ (38 mL, 400 mmol) was added dropwise thereto, and, after raising the temperature to room temperature, the result was stirred for 2 hours. The reaction was terminated with distilled water, and the result was extracted with dichloromethane and dried with MgSO₄. The result went through column purification (MC:HX=1:2) to obtain Compound 1-2 (75 g, 80%).

Preparation of Compound 1-3

In a one-neck round bottom flask (one neck r.b.f), a mixture of 3′-bromo-4-chloro-2′-fluoro-[1,1′-biphenyl]-2-ol (75 g, 248.7 mmol), Cs₂CO₃ (438.7 g, 1243.5 mmol) and dimethylacetamide (750 ml) was stirred at 120° C. The result was cooled, then filtered, and, after removing the solvent of the filtrate, went through column purification (HX:MC=5:1) to obtain Compound 1-3 (79.5 g, 88%).

Preparation of Compound 1-4

In a one-neck round bottom flask (one neck r.b.f), a mixture of 6-bromo-3-chlorodibenzo[b,d]furan) (79.5 g, 282 mmol), bis(pinacolato)diboron (143 g, 564 mmol), Pd (dppf) Cl₂ (20 g, 28.2 mmol), potassium acetate (83 g, 846 mmol) and 1,4-dioxane (800 ml) was refluxed at 140° C. The result was extracted with dichloromethane, concentrated, and then treated with dichloromethane/MeOH to obtain Compound 1-4 (95.5 g, 97%).

Preparation of Compound 1-5

In a one-neck round bottom flask (one neck r.b.f), a mixture of 2-(7-chlorodibenzo[b,d]furan-4-yl)-4,4,5,5-tetramethyl-1,3,2-dioxaborolane (10 g, 30 mmol), 3-bromo-9,9-dimethyl-9H-xanthene (10 g, 36 mmol), tetrakis(triphenylphosphine)palladium(0) (3.5 g, 3 mmol), potassium carbonate (12.4 g, 90 mmol) and 1,4-dioxane/water (150 ml/30 ml) was refluxed for 3 hours at 120° C. The result was filtered at 120° C., and then washed with 1,4-dioxane, distilled water and MeOH to obtain Compound 1-5 (13.4 g, 92%).

Preparation of Compound 1-6

In a one-neck round bottom flask (one neck r.b.f), a mixture of 3-(7-chlorodibenzo[b,d]furan-4-yl)-9,9-dimethyl-9H-xanthene (10 g, 24.3 mmol), bis(pinacolato)diboron (12.3 g, 48 mmol), XPhos (2.3 g, 4.8 mmol), potassium acetate (7.1 g, 73 mmol), Pd₂(dba)₃ (2.2 g, 2.4 mmol) and 1,4-dioxane (100 ml) was refluxed at 140° C. The result was extracted with dichloromethane, concentrated, and then treated with dichloromethane/MeOH to obtain Compound 1-6 (12.7 g, 96%).

Preparation of Compound 1

In a one-neck round bottom flask (one neck r.b.f), a mixture of 2-(6-(9,9-dimethyl-9H-xanthen-3-yl)dibenzo[b,d]furan-3-yl)-4,4,5,5-tetramethyl-1,3,2-dioxaborolane (10 g, 20 mmol), 2-chloro-4,6-diphenyl-1,3,5-triazine (6.4 g, 24 mmol), tetrakis(triphenylphosphine)palladium(0) (2.3 g, 2 mmol), potassium carbonate (8.2 g, 60 mmol) and 1,4-dioxane/water (150 ml/30 ml) was refluxed for 3 hours at 120° C. The result was filtered at 120° C., and then washed with 1,4-dioxane, distilled water and MeOH to obtain Compound 1 (12.7 g, 95%).

The following compounds were synthesized in the same manner as in Preparation Example 1 except that Intermediates A and B of the following Table 1 were used instead of using A and B.

TABLE 1

| | | | | Yield |
|---|---|---|---|---|
| | | | | (%) |
| | | | | (1-4 |
| Compound | A | B | C | to C) |
| 2 | 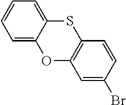 [SMILES: Brc1ccc2c(c1)Oc1ccccc1S2] | 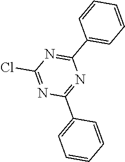 [SMILES: Clc1nc(-c2ccccc2)nc(-c2ccccc2)n1] | 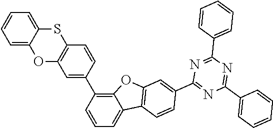 [SMILES: c1ccc(-c2nc(-c3ccccc3)nc(-c3ccc4c(c3)oc3c(-c5ccc6c(c5)Oc5ccccc5S6)cccc34)n2)cc1] | 71 |
| 3 | 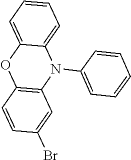 [SMILES: Brc1ccc2c(c1)N(c1ccccc1)c1ccccc1O2] | 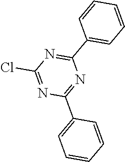 [SMILES: Clc1nc(-c2ccccc2)nc(-c2ccccc2)n1] | 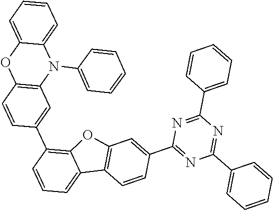 [SMILES: c1ccc(-c2nc(-c3ccccc3)nc(-c3ccc4c(c3)oc3c(-c5ccc6c(c5)N(c5ccccc5)c5ccccc5O6)cccc34)n2)cc1] | 74 |
| 4 | 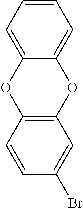 [SMILES: Brc1ccc2c(c1)Oc1ccccc1O2] | 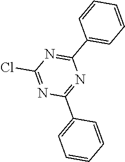 [SMILES: Clc1nc(-c2ccccc2)nc(-c2ccccc2)n1] | 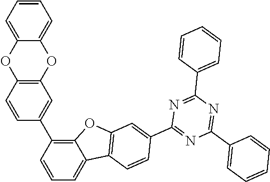 [SMILES: c1ccc(-c2nc(-c3ccccc3)nc(-c3ccc4c(c3)oc3c(-c5ccc6c(c5)Oc5ccccc5O6)cccc34)n2)cc1] | 67 |
| 9 | 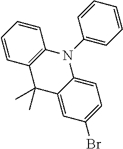 [SMILES: CC1(C)c2ccccc2N(c2ccccc2)c2ccc(Br)cc21] | 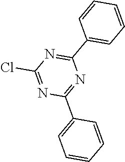 [SMILES: Clc1nc(-c2ccccc2)nc(-c2ccccc2)n1] | 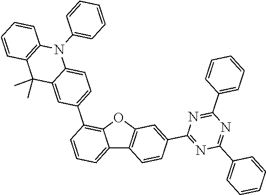 [SMILES: CC1(C)c2ccccc2N(c2ccccc2)c2ccc(-c3cccc4c3oc3cc(-c5nc(-c6ccccc6)nc(-c6ccccc6)n5)ccc34)cc21] | 66 |
| 10 | 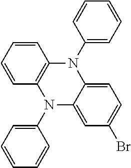 [SMILES: Brc1ccc2c(c1)N(c1ccccc1)c1ccccc1N2c1ccccc1] | 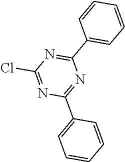 [SMILES: Clc1nc(-c2ccccc2)nc(-c2ccccc2)n1] | 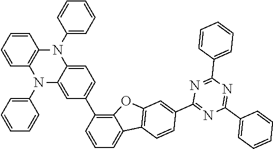 [SMILES: c1ccc(-c2nc(-c3ccccc3)nc(-c3ccc4c(c3)oc3c(-c5ccc6c(c5)N(c5ccccc5)c5ccccc5N6c5ccccc5)cccc34)n2)cc1] | 70 |
| 11 | 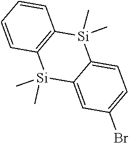 [SMILES: C[Si]1(C)c2ccccc2[Si](C)(C)c2cc(Br)ccc21] | 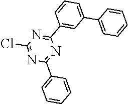 [SMILES: Clc1nc(-c2ccccc2)nc(-c2cccc(-c3ccccc3)c2)n1] | 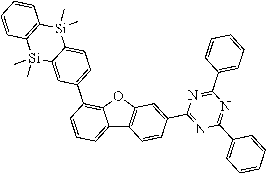 [SMILES: C[Si]1(C)c2ccccc2[Si](C)(C)c2cc(-c3cccc4c3oc3cc(-c5nc(-c6ccccc6)nc(-c6ccccc6)n5)ccc34)ccc21] | 66 |
| 12 | 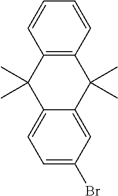 | 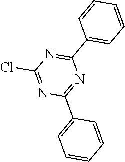 [SMILES: Clc1nc(-c2ccccc2)nc(-c2ccccc2)n1] | 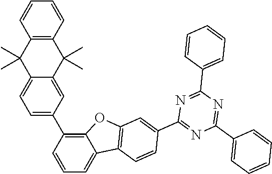 [SMILES: CC1(C)c2ccccc2C(C)(C)c2cc(-c3cccc4c3oc3cc(-c5nc(-c6ccccc6)nc(-c6ccccc6)n5)ccc34)ccc21] | 78 |
| 13 | 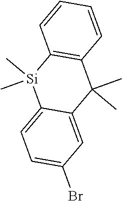 | 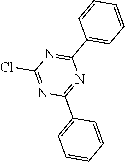 [SMILES: Clc1nc(-c2ccccc2)nc(-c2ccccc2)n1] | 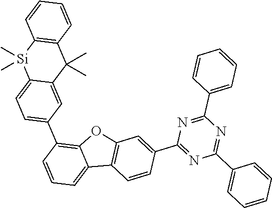 [SMILES: CC1(C)c2ccccc2[Si](C)(C)c2ccc(-c3cccc4c3oc3cc(-c5nc(-c6ccccc6)nc(-c6ccccc6)n5)ccc34)cc21] | 62 |
| 14 | 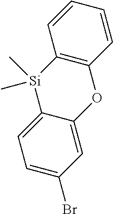 [SMILES: C[Si]1(C)c2ccccc2Oc2cc(Br)ccc21] | 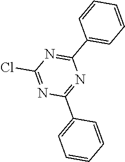 [SMILES: Clc1nc(-c2ccccc2)nc(-c2ccccc2)n1] | 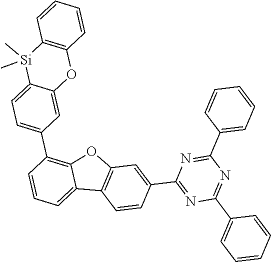 [SMILES: C[Si]1(C)c2ccccc2Oc2cc(-c3cccc4c3oc3cc(-c5nc(-c6ccccc6)nc(-c6ccccc6)n5)ccc34)ccc21] | 81 |
| 15 | 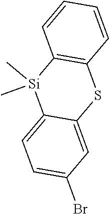 [SMILES: C[Si]1(C)c2ccccc2Sc2cc(Br)ccc21] | 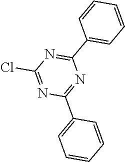 [SMILES: Clc1nc(-c2ccccc2)nc(-c2ccccc2)n1] | 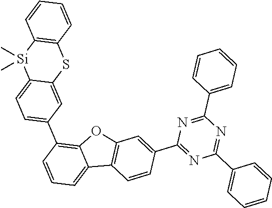 [SMILES: C[Si]1(C)c2ccccc2Sc2cc(-c3cccc4c3oc3cc(-c5nc(-c6ccccc6)nc(-c6ccccc6)n5)ccc34)ccc21] | 79 |
| 16 | 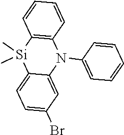 [SMILES: C[Si]1(C)c2ccccc2N(c2ccccc2)c2cc(Br)ccc21] | 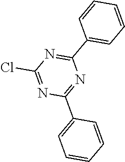 [SMILES: Clc1nc(-c2ccccc2)nc(-c2ccccc2)n1] | 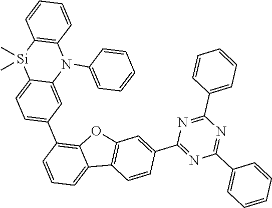 [SMILES: C[Si]1(C)c2ccccc2N(c2ccccc2)c2cc(-c3cccc4c3oc3cc(-c5nc(-c6ccccc6)nc(-c6ccccc6)n5)ccc34)ccc21] | 76 |
| 17 | 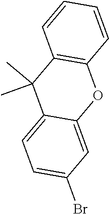 [SMILES: CC1(C)c2ccccc2Oc2cc(Br)ccc21] | 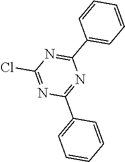 [SMILES: Clc1nc(-c2ccccc2)nc(-c2ccccc2)n1] | 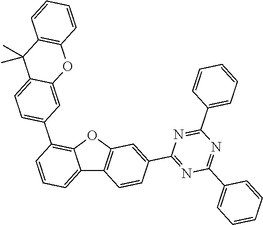 [SMILES: CC1(C)c2ccccc2Oc2cc(-c3cccc4c3oc3cc(-c5nc(-c6ccccc6)nc(-c6ccccc6)n5)ccc34)ccc21] | 65 |
| 18 | 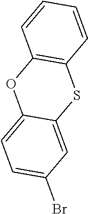 [SMILES: Brc1ccc2c(c1)Sc1ccccc1O2] | 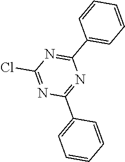 [SMILES: Clc1nc(-c2ccccc2)nc(-c2ccccc2)n1] | 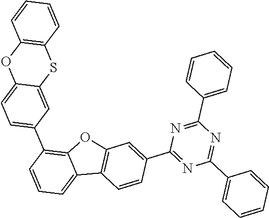 [SMILES: c1ccc(-c2nc(-c3ccccc3)nc(-c3ccc4c(c3)oc3c(-c5ccc6c(c5)Sc5ccccc5O6)cccc34)n2)cc1] | 69 |
| 19 | 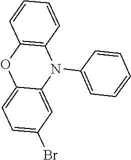 [SMILES: Brc1ccc2c(c1)N(c1ccccc1)c1ccccc1O2] | 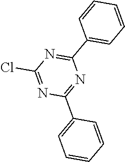 [SMILES: Clc1nc(-c2ccccc2)nc(-c2ccccc2)n1] | 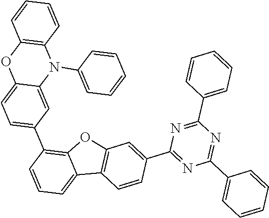 [SMILES: c1ccc(-c2nc(-c3ccccc3)nc(-c3ccc4c(c3)oc3c(-c5ccc6c(c5)N(c5ccccc5)c5ccccc5O6)cccc34)n2)cc1] | 72 |
| 20 | 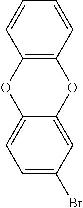 [SMILES: Brc1ccc2c(c1)Oc1ccccc1O2] | 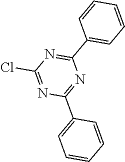 [SMILES: Clc1nc(-c2ccccc2)nc(-c2ccccc2)n1] | 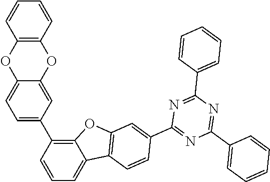 [SMILES: c1ccc(-c2nc(-c3ccccc3)nc(-c3ccc4c(c3)oc3c(-c5ccc6c(c5)Oc5ccccc5O6)cccc34)n2)cc1] | 68 |
| 21 | 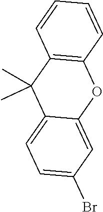 [SMILES: CC1(C)c2ccccc2Oc2cc(Br)ccc21] | 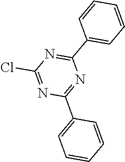 [SMILES: Clc1nc(-c2ccccc2)nc(-c2ccccc2)n1] | 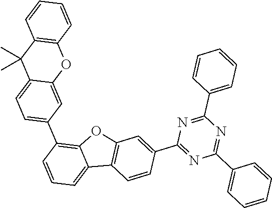 [SMILES: CC1(C)c2ccccc2Oc2cc(-c3cccc4c3oc3cc(-c5nc(-c6ccccc6)nc(-c6ccccc6)n5)ccc34)ccc21] | 74 |
| 22 | 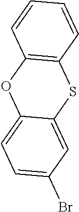 [SMILES: Brc1ccc2c(c1)Sc1ccccc1O2] | 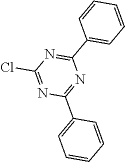 [SMILES: Clc1nc(-c2ccccc2)nc(-c2ccccc2)n1] | 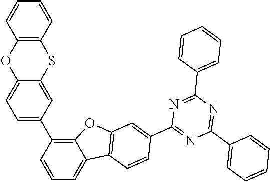 [SMILES: c1ccc(-c2nc(-c3ccccc3)nc(-c3ccc4c(c3)oc3c(-c5ccc6c(c5)Sc5ccccc5O6)cccc34)n2)cc1] | 75 |
| 23 | 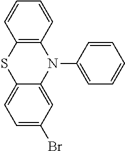 [SMILES: Brc1ccc2c(c1)N(c1ccccc1)c1ccccc1S2] | 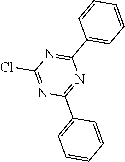 [SMILES: Clc1nc(-c2ccccc2)nc(-c2ccccc2)n1] | 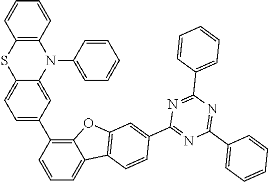 [SMILES: c1ccc(-c2nc(-c3ccccc3)nc(-c3ccc4c(c3)oc3c(-c5ccc6c(c5)N(c5ccccc5)c5ccccc5S6)cccc34)n2)cc1] | 70 |
| 24 | 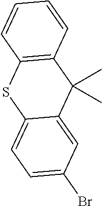 [SMILES: CC1(C)c2ccccc2Sc2ccc(Br)cc21] | 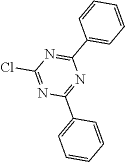 [SMILES: Clc1nc(-c2ccccc2)nc(-c2ccccc2)n1] | 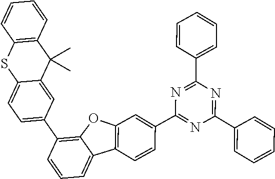 [SMILES: CC1(C)c2ccccc2Sc2ccc(-c3cccc4c3oc3cc(-c5nc(-c6ccccc6)nc(-c6ccccc6)n5)ccc34)cc21] | 70 |
| 25 | 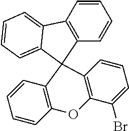 [SMILES: Brc1cccc2c1Oc1ccccc1C21c2ccccc2-c2ccccc21] | 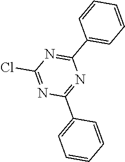 [SMILES: Clc1nc(-c2ccccc2)nc(-c2ccccc2)n1] | 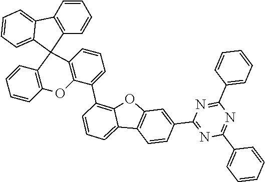 [SMILES: c1ccc(-c2nc(-c3ccccc3)nc(-c3ccc4c(c3)oc3c(-c5cccc6c5Oc5ccccc5C65c6ccccc6-c6ccccc65)cccc34)n2)cc1] | 78 |
| 26 | 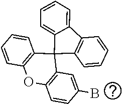 | 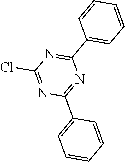 [SMILES: Clc1nc(-c2ccccc2)nc(-c2ccccc2)n1] | 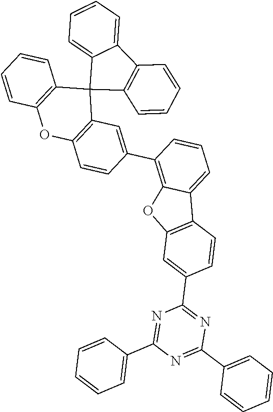 [SMILES: c1ccc(-c2nc(-c3ccccc3)nc(-c3ccc4c(c3)oc3c(-c5ccc6c(c5)C5(c7ccccc7O6)c6ccccc6-c6ccccc65)cccc34)n2)cc1] | 79 |
| 28 | 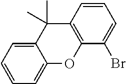 [SMILES: CC1(C)c2ccccc2Oc2c(Br)cccc21] | 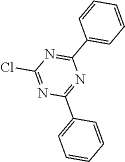 [SMILES: Clc1nc(-c2ccccc2)nc(-c2ccccc2)n1] | 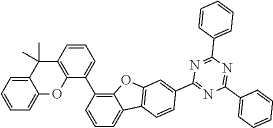 [SMILES: CC1(C)c2ccccc2Oc2c(-c3cccc4c3oc3cc(-c5nc(-c6ccccc6)nc(-c6ccccc6)n5)ccc34)cccc21] | 83 |
| 33 | 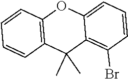 [SMILES: CC1(C)c2ccccc2Oc2cccc(Br)c21] | 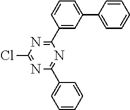 [SMILES: Clc1nc(-c2ccccc2)nc(-c2cccc(-c3ccccc3)c2)n1] | 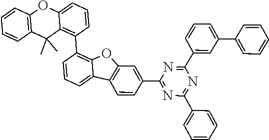 [SMILES: CC1(C)c2ccccc2Oc2cccc(-c3cccc4c3oc3cc(-c5nc(-c6ccccc6)nc(-c6cccc(-c7ccccc7)c6)n5)ccc34)c21] | 88 |
| 34 | 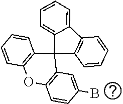 | 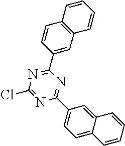 [SMILES: Clc1nc(-c2ccc3ccccc3c2)nc(-c2ccc3ccccc3c2)n1] | 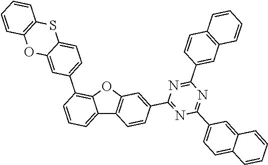 [SMILES: c1ccc2c(c1)Oc1cc(-c3cccc4c3oc3cc(-c5nc(-c6ccc7ccccc7c6)nc(-c6ccc7ccccc7c6)n5)ccc34)ccc1S2] | 88 |
| 35 | 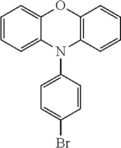 [SMILES: Brc1ccc(N2c3ccccc3Oc3ccccc32)cc1] | 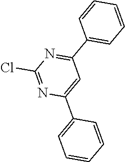 [SMILES: Clc1nc(-c2ccccc2)cc(-c2ccccc2)n1] | 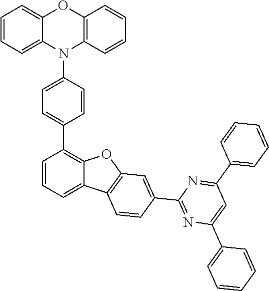 [SMILES: c1ccc(-c2cc(-c3ccccc3)nc(-c3ccc4c(c3)oc3c(-c5ccc(N6c7ccccc7Oc7ccccc76)cc5)cccc34)n2)cc1] | 82 |
| 36 | 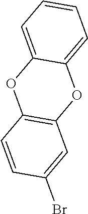 [SMILES: Brc1ccc2c(c1)Oc1ccccc1O2] | 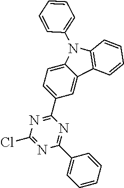 [SMILES: Clc1nc(-c2ccccc2)nc(-c2ccc3c(c2)c2ccccc2n3-c2ccccc2)n1] | 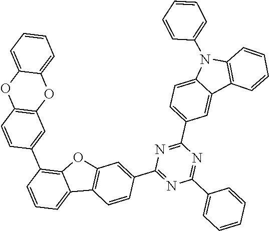 [SMILES: c1ccc(-c2nc(-c3ccc4c(c3)oc3c(-c5ccc6c(c5)Oc5ccccc5O6)cccc34)nc(-c3ccc4c(c3)c3ccccc3n4-c3ccccc3)n2)cc1] | 75 |
| 37 | 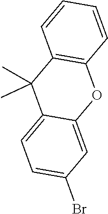 [SMILES: CC1(C)c2ccccc2Oc2cc(Br)ccc21] | 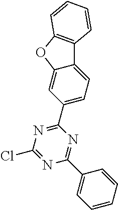 [SMILES: Clc1nc(-c2ccccc2)nc(-c2ccc3c(c2)oc2ccccc23)n1] | 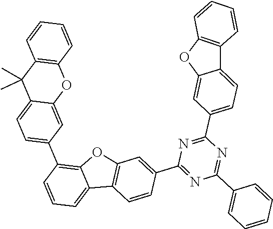 [SMILES: CC1(C)c2ccccc2Oc2cc(-c3cccc4c3oc3cc(-c5nc(-c6ccccc6)nc(-c6ccc7c(c6)oc6ccccc67)n5)ccc34)ccc21] | 77 |
| 38 | 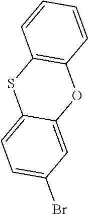 [SMILES: Brc1ccc2c(c1)Oc1ccccc1S2] | 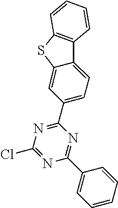 [SMILES: Clc1nc(-c2ccccc2)nc(-c2ccc3c(c2)sc2ccccc23)n1] | 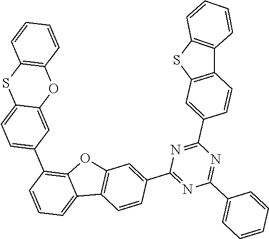 [SMILES: c1ccc(-c2nc(-c3ccc4c(c3)oc3c(-c5ccc6c(c5)Oc5ccccc5S6)cccc34)nc(-c3ccc4c(c3)sc3ccccc34)n2)cc1] | 78 |
| 39 | 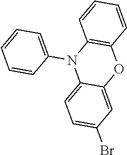 [SMILES: Brc1ccc2c(c1)Oc1ccccc1N2c1ccccc1] | 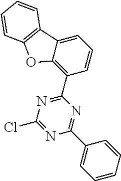 [SMILES: Clc1nc(-c2ccccc2)nc(-c2cccc3c2oc2ccccc23)n1] | 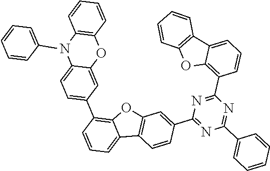 [SMILES: c1ccc(-c2nc(-c3ccc4c(c3)oc3c(-c5ccc6c(c5)Oc5ccccc5N6c5ccccc5)cccc34)nc(-c3cccc4c3oc3ccccc34)n2)cc1] | 77 |
| 40 | 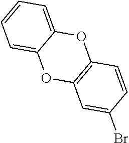 [SMILES: Brc1ccc2c(c1)Oc1ccccc1O2] | 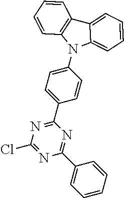 [SMILES: Clc1nc(-c2ccccc2)nc(-c2ccc(-n3c4ccccc4c4ccccc43)cc2)n1] | 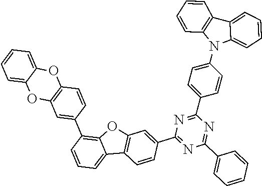 [SMILES: c1ccc(-c2nc(-c3ccc(-n4c5ccccc5c5ccccc54)cc3)nc(-c3ccc4c(c3)oc3c(-c5ccc6c(c5)Oc5ccccc5O6)cccc34)n2)cc1] | 79 |
| 41 | 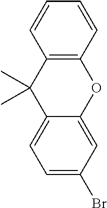 [SMILES: CC1(C)c2ccccc2Oc2cc(Br)ccc21] | 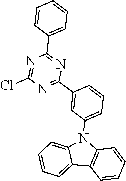 [SMILES: Clc1nc(-c2ccccc2)nc(-c2cccc(-n3c4ccccc4c4ccccc43)c2)n1] | 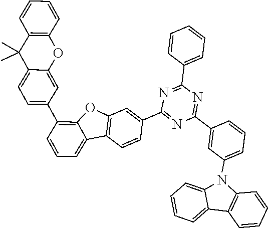 [SMILES: CC1(C)c2ccccc2Oc2cc(-c3cccc4c3oc3cc(-c5nc(-c6ccccc6)nc(-c6cccc(-n7c8ccccc8c8ccccc87)c6)n5)ccc34)ccc21] | 82 |
| 42 | 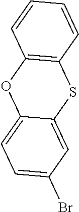 [SMILES: Brc1ccc2c(c1)Sc1ccccc1O2] | 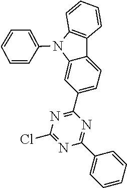 [SMILES: Clc1nc(-c2ccccc2)nc(-c2ccc3c4ccccc4n(-c4ccccc4)c3c2)n1] | 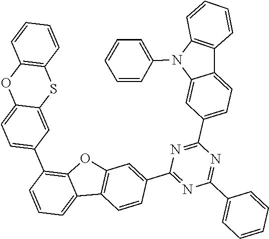 [SMILES: c1ccc(-c2nc(-c3ccc4c(c3)oc3c(-c5ccc6c(c5)Sc5ccccc5O6)cccc34)nc(-c3ccc4c5ccccc5n(-c5ccccc5)c4c3)n2)cc1] | 83 |
| 43 | 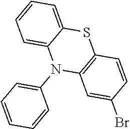 [SMILES: Brc1ccc2c(c1)N(c1ccccc1)c1ccccc1S2] | 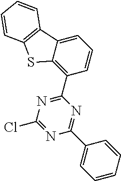 [SMILES: Clc1nc(-c2ccccc2)nc(-c2cccc3c2sc2ccccc23)n1] | 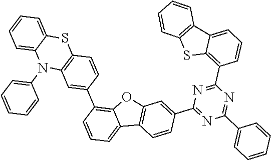 [SMILES: c1ccc(-c2nc(-c3ccc4c(c3)oc3c(-c5ccc6c(c5)N(c5ccccc5)c5ccccc5S6)cccc34)nc(-c3cccc4c3sc3ccccc34)n2)cc1] | 85 |
| 44 | 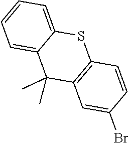 [SMILES: CC1(C)c2ccccc2Sc2ccc(Br)cc21] | 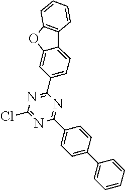 [SMILES: Clc1nc(-c2ccc(-c3ccccc3)cc2)nc(-c2ccc3c(c2)oc2ccccc23)n1] | 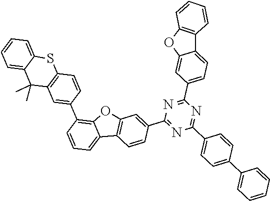 [SMILES: CC1(C)c2ccccc2Sc2ccc(-c3cccc4c3oc3cc(-c5nc(-c6ccc(-c7ccccc7)cc6)nc(-c6ccc7c(c6)oc6ccccc67)n5)ccc34)cc21] | 75 |
| 45 | 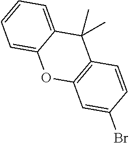 [SMILES: CC1(C)c2ccccc2Oc2cc(Br)ccc21] | 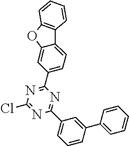 [SMILES: Clc1nc(-c2cccc(-c3ccccc3)c2)nc(-c2ccc3c(c2)oc2ccccc23)n1] | 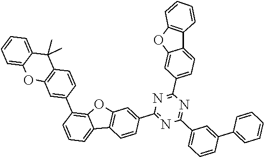 [SMILES: CC1(C)c2ccccc2Oc2cc(-c3cccc4c3oc3cc(-c5nc(-c6cccc(-c7ccccc7)c6)nc(-c6ccc7c(c6)oc6ccccc67)n5)ccc34)ccc21] | 76 |
| 46 | 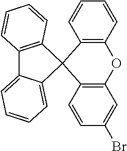 [SMILES: Brc1ccc2c(c1)Oc1ccccc1C21c2ccccc2-c2ccccc21] | 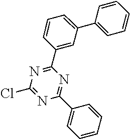 [SMILES: Clc1nc(-c2ccccc2)nc(-c2cccc(-c3ccccc3)c2)n1] | 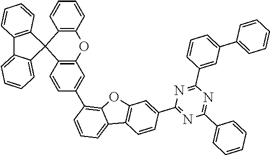 [SMILES: c1ccc(-c2cccc(-c3nc(-c4ccccc4)nc(-c4ccc5c(c4)oc4c(-c6ccc7c(c6)Oc6ccccc6C76c7ccccc7-c7ccccc76)cccc45)n3)c2)cc1] | 76 |

### [Preparation Example 2] Preparation of Compound 5(F)

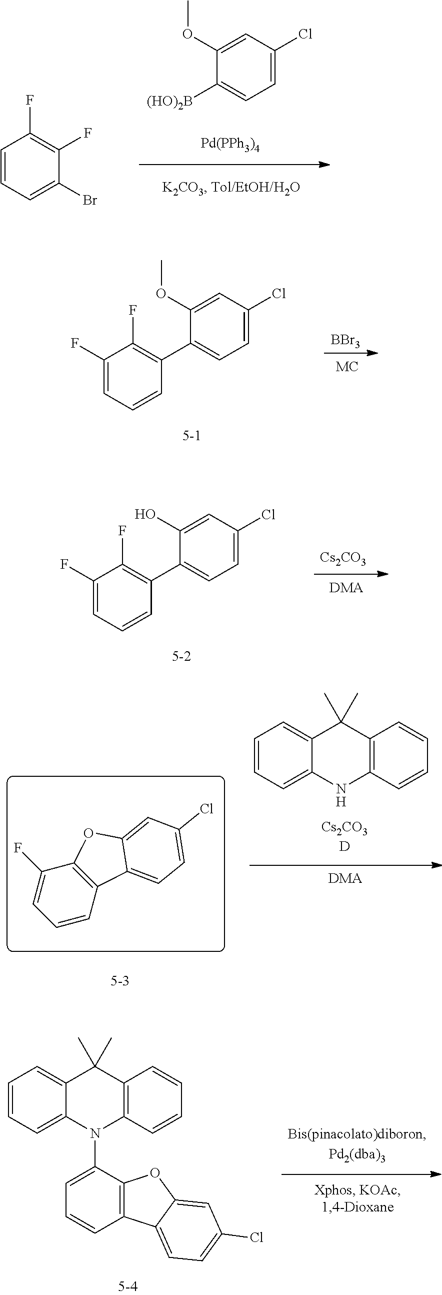

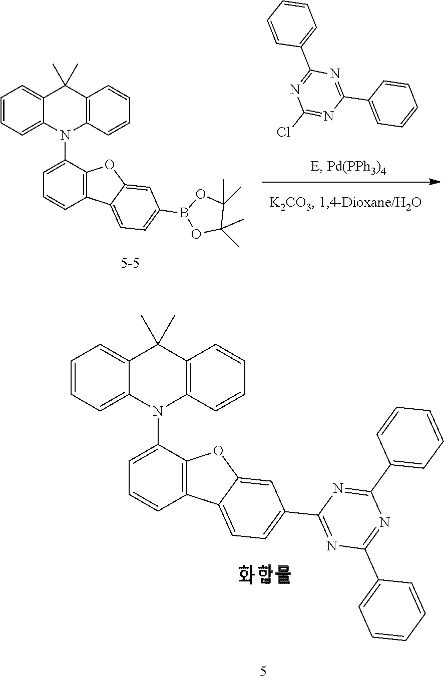 [SMILES: CC1(C)c2ccccc2N(c2cccc3c2oc2cc(B4OC(C)(C)C(C)(C)O4)ccc23)c2ccccc21 ; Clc1nc(-c2ccccc2)nc(-c2ccccc2)n1 ; CC1(C)c2ccccc2N(c2cccc3c2oc2cc(-c4nc(-c5ccccc5)nc(-c5ccccc5)n4)ccc23)c2ccccc21]

Preparation of Compound 5-1

In a one-neck round bottom flask (one neck r.b.f), a mixture of 1-bromo-2,3-difluorobenzene (50 g, 259 mmol), (4-chloro-2-methoxyphenyl)boronic acid (57.7 g, 310 mmol), tetrakis(triphenylphosphine)palladium(0) (29 g, 25.9 mmol), potassium carbonate (71.5 g, 51.8 mol) and toluene/ethanol/water (800 ml/160 ml/160 ml) was refluxed at 110° C. The result was extracted with dichloromethane and dried with MgSO₄. The result was silica gel filtered and then concentrated to obtain Compound 5-1 (65 g, 99%).

Preparation of Compound 5-2

In a one-neck round bottom flask (one neck r.b.f), a mixture of 4′-chloro-2,3-difluoro-2′-methoxy-1,1′-biphenyl (65 g, 255 mmol) and MC (1000 ml) was cooled to a temperature of 0° C., BBr₃ (48 mL, 500 mmol) was added dropwise thereto, and, after raising the temperature to room temperature, the result was stirred for 2 hours.

The reaction was terminated with distilled water, and the result was extracted with dichloromethane and dried with MgSO₄. The result went through column purification (MC:HX=1:2) to obtain Compound 5-2 (49 g, 80%).

Preparation of Compound 5-3

In a one-neck round bottom flask (one neck r.b.f), a mixture of 4-chloro-2′,3′-difluoro-[1,1′-biphenyl]-2-ol (49 g, 203 mmol), Cs₂CO₃ (331 g, 1018 mmol) and dimethylacetamide (500 ml) was stirred at 120° C. The result was cooled, then filtered, and, after removing the solvent of the filtrate, went through column purification (HX:MC=5:1) to obtain Compound 5-3 (50.1 g, 88%).

Preparation of Compound 5-4

In a one-neck round bottom flask (one neck r.b.f), a mixture of 3-chloro-6-fluorodibenzo[b,d]furan (10 g, 45 mmol), 9,9-dimethyl-9,10-dihydroacridine (11.4 g, 54.3 mmol), Cs₂CO₃ (31.7 g, 90 mmol) and dimethylacetamide (100 ml) was refluxed for 12 hours at 170° C. The result was cooled, then filtered, and, after removing the solvent of the filtrate, went through column purification (HX:MC=4:1) to obtain Compound 5-4 (27.5 g, 67%).

Preparation of Compound 5-5

In a one-neck round bottom flask (one neck r.b.f), a mixture of 10-(7-chlorodibenzo[b,d]furan-4-yl)-9,9-dimethyl-9,10-dihydroacridine (10 g, 24.4 mmol), bis(pinacolato)diboron (12.3 g, 48.8 mmol), XPhos (2.3 g, 4.8 mmol), potassium acetate (7.1 g, 73.2 mmol), Pd₂(dba)₃ (2.2 g, 2.4 mmol) and 1,4-dioxane (100 ml) was refluxed at 140° C. The result was extracted with dichloromethane, concentrated, and then treated with dichloromethane/MeOH to obtain Compound 5-5 (12.5 g, 98%).

Preparation of Compound 5

In a one-neck round bottom flask (one neck r.b.f), a mixture of 9,9-dimethyl-10-(7-(4,4,5,5-tetramethyl-1,3,2-dioxaborolan-2-yl)dibenzo[b,d]furan-4-yl)-9,10-dihydroacridine (10 g, 20 mmol), 2-chloro-4,6-diphenyl-1,3,5-triazine (6.4 g, 24 mmol), tetrakis(triphenylphosphine)palladium(0) (2.3 g, 2 mmol), potassium carbonate (8.3 g, 60 mmol) and 1,4-dioxane/water (150 ml/30 ml) was refluxed for 3 hours at 120° C. The result was filtered at 120° C., and then washed with 1,4-dioxane, distilled water and MeOH to obtain Compound 5 (14.8 g, 82%).

The following compounds were synthesized in the same manner as in Preparation Example 2 except that Intermediates D and E of the following Table 2 were used instead of using D and E.

TABLE 2

| | | | | Yield |
|---|---|---|---|---|
| | | | | (%) |
| | | | | (5-3 |
| Compound | D | E | F | to F) |
| 6 | 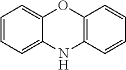 [SMILES: c1ccc2c(c1)Nc1ccccc1O2] | 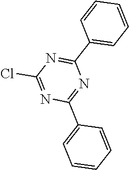 [SMILES: Clc1nc(-c2ccccc2)nc(-c2ccccc2)n1] | 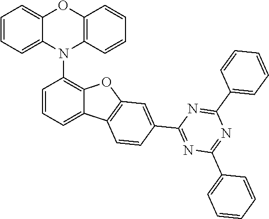 [SMILES: c1ccc(-c2nc(-c3ccccc3)nc(-c3ccc4c(c3)oc3c(N5c6ccccc6Oc6ccccc65)cccc34)n2)cc1] | 75 |
| 7 | 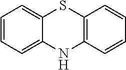 [SMILES: c1ccc2c(c1)Nc1ccccc1S2] | 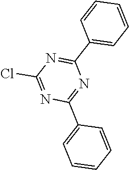 [SMILES: Clc1nc(-c2ccccc2)nc(-c2ccccc2)n1] | 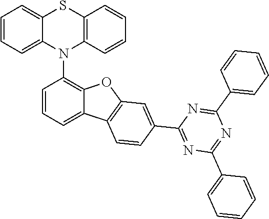 [SMILES: c1ccc(-c2nc(-c3ccccc3)nc(-c3ccc4c(c3)oc3c(N5c6ccccc6Sc6ccccc65)cccc34)n2)cc1] | 86 |
| 8 | 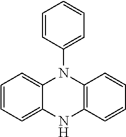 [SMILES: c1ccc(N2c3ccccc3Nc3ccccc32)cc1] | 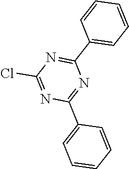 [SMILES: Clc1nc(-c2ccccc2)nc(-c2ccccc2)n1] | 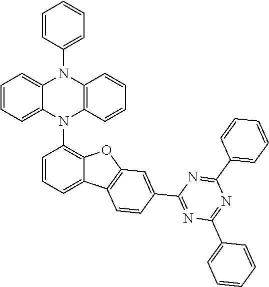 [SMILES: c1ccc(-c2nc(-c3ccccc3)nc(-c3ccc4c(c3)oc3c(N5c6ccccc6N(c6ccccc6)c6ccccc65)cccc34)n2)cc1] | 73 |
| 27 | 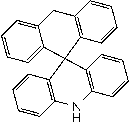 [SMILES: c1ccc2c(c1)Cc1ccccc1C21c2ccccc2Nc2ccccc21] | 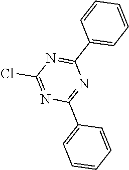 [SMILES: Clc1nc(-c2ccccc2)nc(-c2ccccc2)n1] | 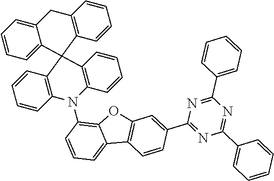 [SMILES: c1ccc(-c2nc(-c3ccccc3)nc(-c3ccc4c(c3)oc3c(N5c6ccccc6C6(c7ccccc7Cc7ccccc76)c6ccccc65)cccc34)n2)cc1] | 78 |
| 29 | 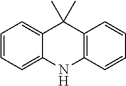 [SMILES: CC1(C)c2ccccc2Nc2ccccc21] | 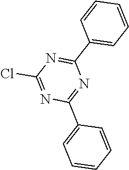 [SMILES: Clc1nc(-c2ccccc2)nc(-c2ccccc2)n1] | 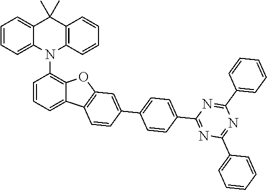 [SMILES: CC1(C)c2ccccc2N(c2cccc3c2oc2cc(-c4ccc(-c5nc(-c6ccccc6)nc(-c6ccccc6)n5)cc4)ccc23)c2ccccc21] | 77 |
| 30 | 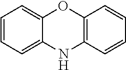 [SMILES: c1ccc2c(c1)Nc1ccccc1O2] | 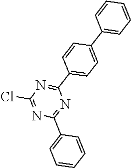 [SMILES: Clc1nc(-c2ccccc2)nc(-c2ccc(-c3ccccc3)cc2)n1] | 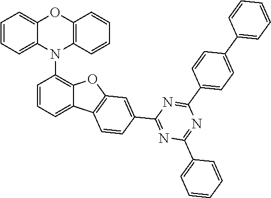 [SMILES: c1ccc(-c2ccc(-c3nc(-c4ccccc4)nc(-c4ccc5c(c4)oc4c(N6c7ccccc7Oc7ccccc76)cccc45)n3)cc2)cc1] | 79 |
| 31 | 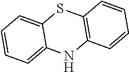 [SMILES: c1ccc2c(c1)Nc1ccccc1S2] | 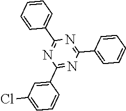 [SMILES: Clc1cccc(-c2nc(-c3ccccc3)nc(-c3ccccc3)n2)c1] | 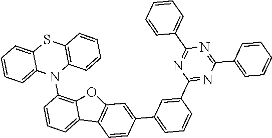 [SMILES: c1ccc(-c2nc(-c3ccccc3)nc(-c3cccc(-c4ccc5c(c4)oc4c(N6c7ccccc7Sc7ccccc76)cccc45)c3)n2)cc1] | 63 |
| 32 | 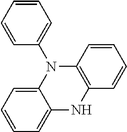 [SMILES: c1ccc(N2c3ccccc3Nc3ccccc32)cc1] | 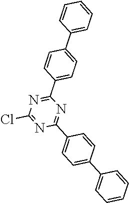 [SMILES: Clc1nc(-c2ccc(-c3ccccc3)cc2)nc(-c2ccc(-c3ccccc3)cc2)n1] | 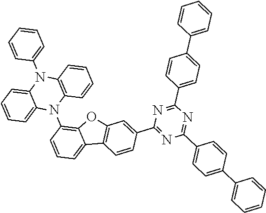 [SMILES: c1ccc(-c2ccc(-c3nc(-c4ccc(-c5ccccc5)cc4)nc(-c4ccc5c(c4)oc4c(N6c7ccccc7N(c7ccccc7)c7ccccc76)cccc45)n3)cc2)cc1] | 78 |
| 47 | 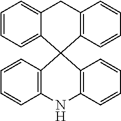 [SMILES: c1ccc2c(c1)Cc1ccccc1C21c2ccccc2Nc2ccccc21] | 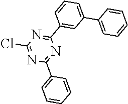 [SMILES: Clc1nc(-c2ccccc2)nc(-c2cccc(-c3ccccc3)c2)n1] | 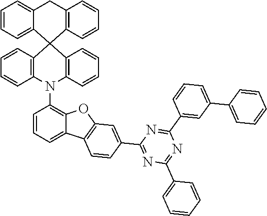 [SMILES: c1ccc(-c2cccc(-c3nc(-c4ccccc4)nc(-c4ccc5c(c4)oc4c(N6c7ccccc7C7(c8ccccc8Cc8ccccc87)c7ccccc76)cccc45)n3)c2)cc1] | 77 |
| 48 | 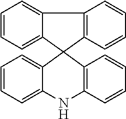 [SMILES: c1ccc2c(c1)Nc1ccccc1C21c2ccccc2-c2ccccc21] | 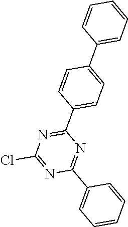 [SMILES: Clc1nc(-c2ccccc2)nc(-c2ccc(-c3ccccc3)cc2)n1] | 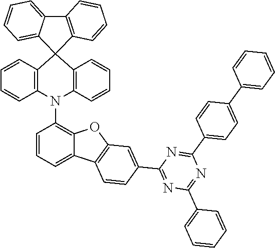 [SMILES: c1ccc(-c2ccc(-c3nc(-c4ccccc4)nc(-c4ccc5c(c4)oc4c(N6c7ccccc7C7(c8ccccc8-c8ccccc87)c7ccccc76)cccc45)n3)cc2)cc1] | 75 |

### [Preparation Example 3] Preparation of Compound 49(G)

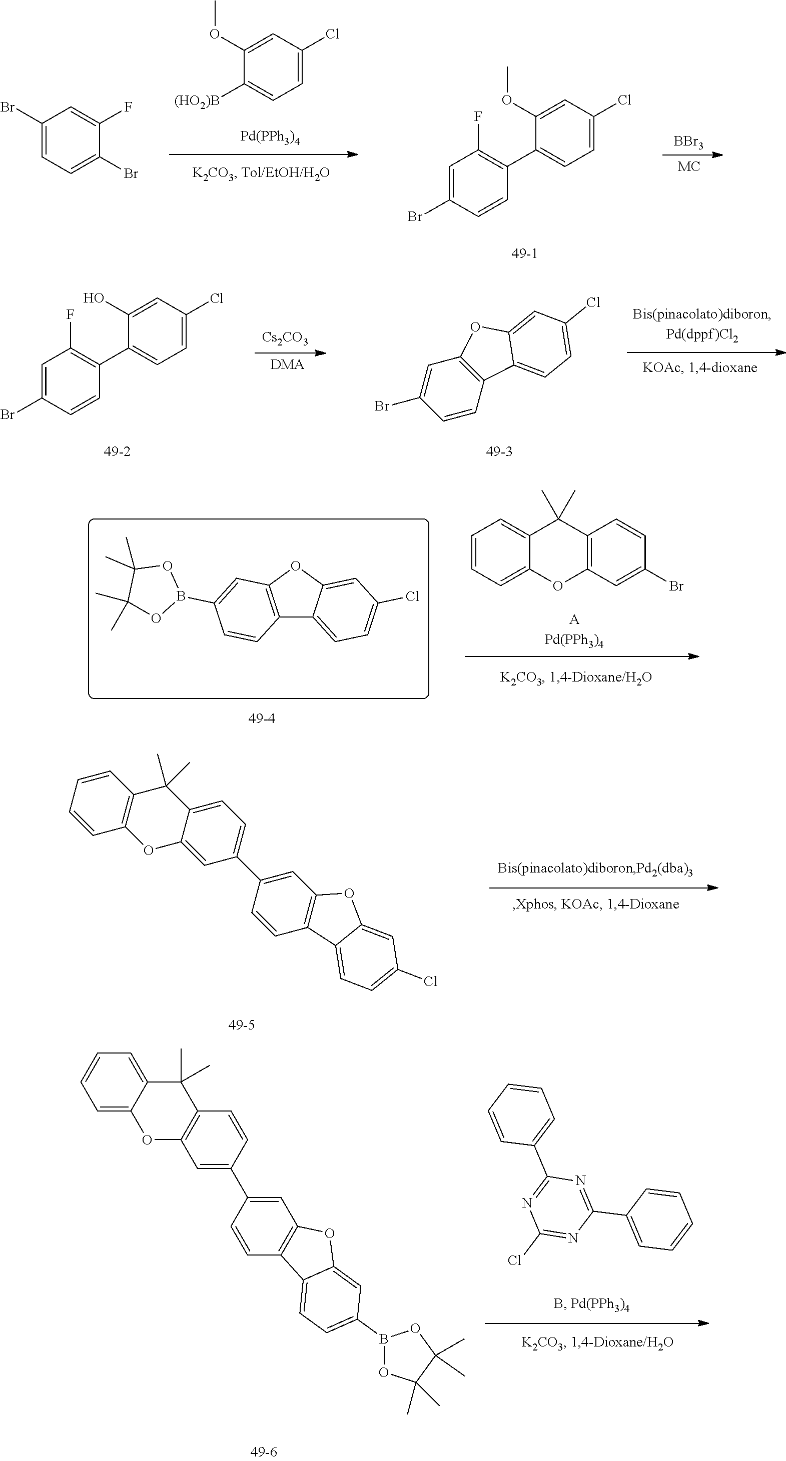

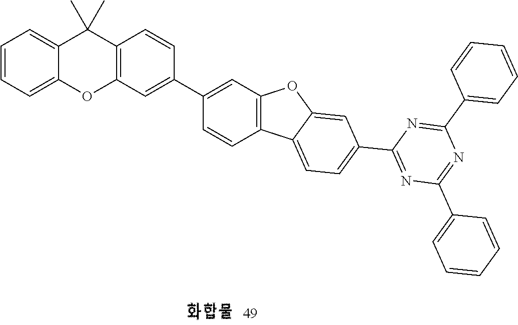 [SMILES: CC1(C)c2ccccc2Oc2cc(-c3ccc4c(c3)oc3cc(-c5nc(-c6ccccc6)nc(-c6ccccc6)n5)ccc34)ccc21]

Target Compound 49 (G) (12.7 g, 95%) was obtained in the same manner as in Preparation of Compound 1 of Preparation Example 1 except that 1,4-dibromo-2-fluorobenzene was used instead of 1,3-dibromo-2-fluorobenzene

The following compounds were synthesized in the same manner as in Preparation Example 3 except that Intermediates A and B of the following Table 3 were used instead of using A and B.

TABLE 3

| | | | | Yield |
|---|---|---|---|---|
| | | | | (%) |
| | | | | (49-4 |
| Compound | A | B | C | to C) |
| 50 | 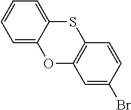 [SMILES: Brc1ccc2c(c1)Oc1ccccc1S2] | 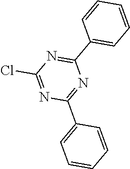 [SMILES: Clc1nc(-c2ccccc2)nc(-c2ccccc2)n1] | 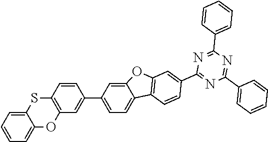 [SMILES: c1ccc(-c2nc(-c3ccccc3)nc(-c3ccc4c(c3)oc3cc(-c5ccc6c(c5)Oc5ccccc5S6)ccc34)n2)cc1] | 71 |
| 51 | 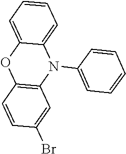 [SMILES: Brc1ccc2c(c1)N(c1ccccc1)c1ccccc1O2] | 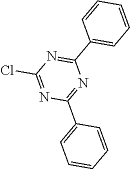 [SMILES: Clc1nc(-c2ccccc2)nc(-c2ccccc2)n1] | 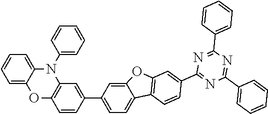 [SMILES: c1ccc(-c2nc(-c3ccccc3)nc(-c3ccc4c(c3)oc3cc(-c5ccc6c(c5)N(c5ccccc5)c5ccccc5O6)ccc34)n2)cc1] | 74 |
| 52 | 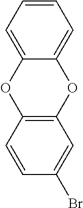 [SMILES: Brc1ccc2c(c1)Oc1ccccc1O2] | 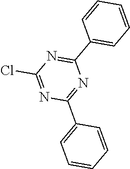 [SMILES: Clc1nc(-c2ccccc2)nc(-c2ccccc2)n1] | 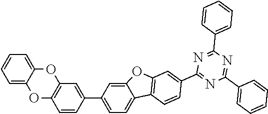 [SMILES: c1ccc(-c2nc(-c3ccccc3)nc(-c3ccc4c(c3)oc3cc(-c5ccc6c(c5)Oc5ccccc5O6)ccc34)n2)cc1] | 76 |
| 57 | 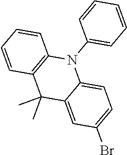 [SMILES: CC1(C)c2ccccc2N(c2ccccc2)c2ccc(Br)cc21] | 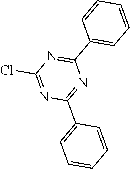 [SMILES: Clc1nc(-c2ccccc2)nc(-c2ccccc2)n1] | 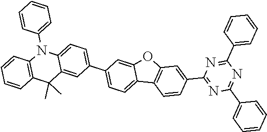 [SMILES: CC1(C)c2ccccc2N(c2ccccc2)c2ccc(-c3ccc4c(c3)oc3cc(-c5nc(-c6ccccc6)nc(-c6ccccc6)n5)ccc34)cc21] | 66 |
| 58 | 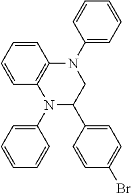 [SMILES: Brc1ccc(C2CN(c3ccccc3)c3ccccc3N2c2ccccc2)cc1] | 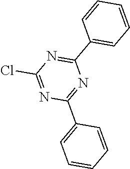 [SMILES: Clc1nc(-c2ccccc2)nc(-c2ccccc2)n1] | 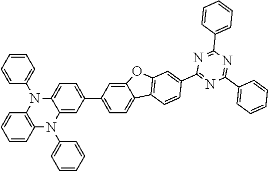 [SMILES: c1ccc(-c2nc(-c3ccccc3)nc(-c3ccc4c(c3)oc3cc(-c5ccc6c(c5)N(c5ccccc5)c5ccccc5N6c5ccccc5)ccc34)n2)cc1] | 75 |
| 59 | 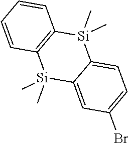 [SMILES: C[Si]1(C)c2ccccc2[Si](C)(C)c2cc(Br)ccc21] | 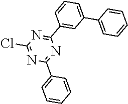 [SMILES: Clc1nc(-c2ccccc2)nc(-c2cccc(-c3ccccc3)c2)n1] | 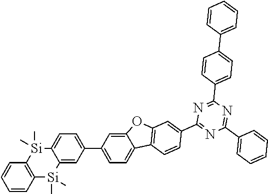 [SMILES: C[Si]1(C)c2ccccc2[Si](C)(C)c2cc(-c3ccc4c(c3)oc3cc(-c5nc(-c6ccccc6)nc(-c6ccc(-c7ccccc7)cc6)n5)ccc34)ccc21] | 70 |

### [Preparation Example 4] Preparation of Compound 97 (H)

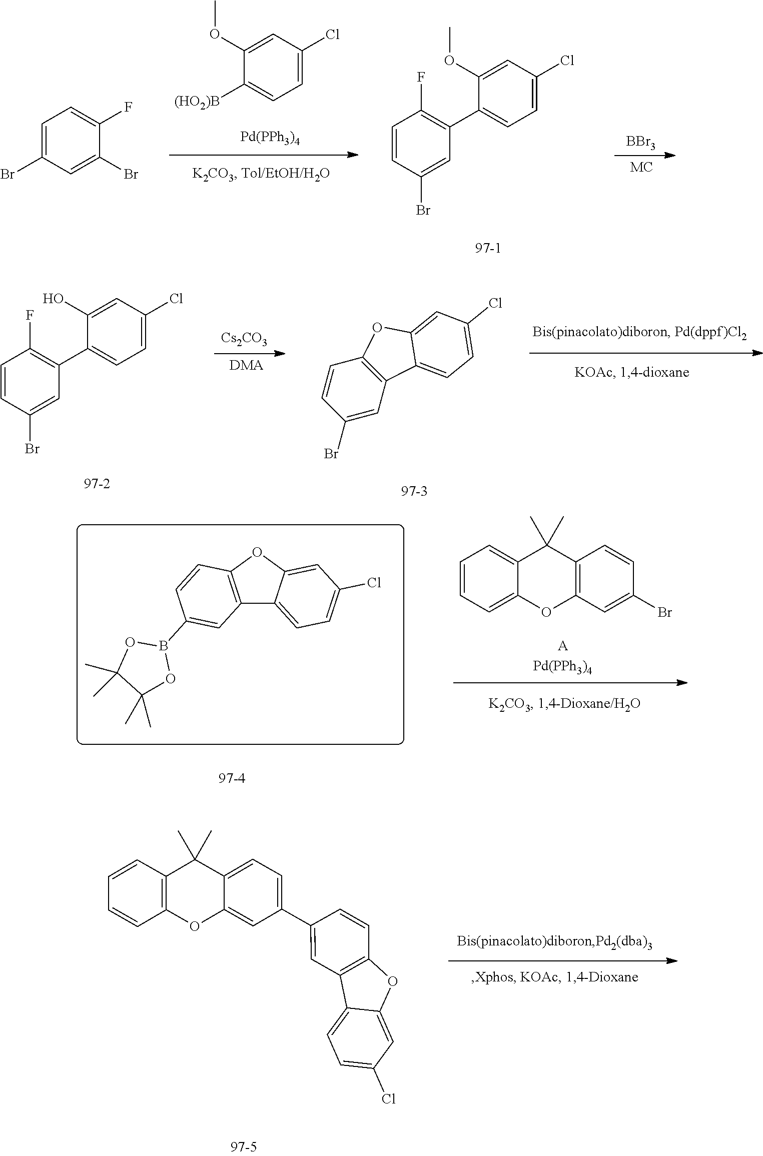

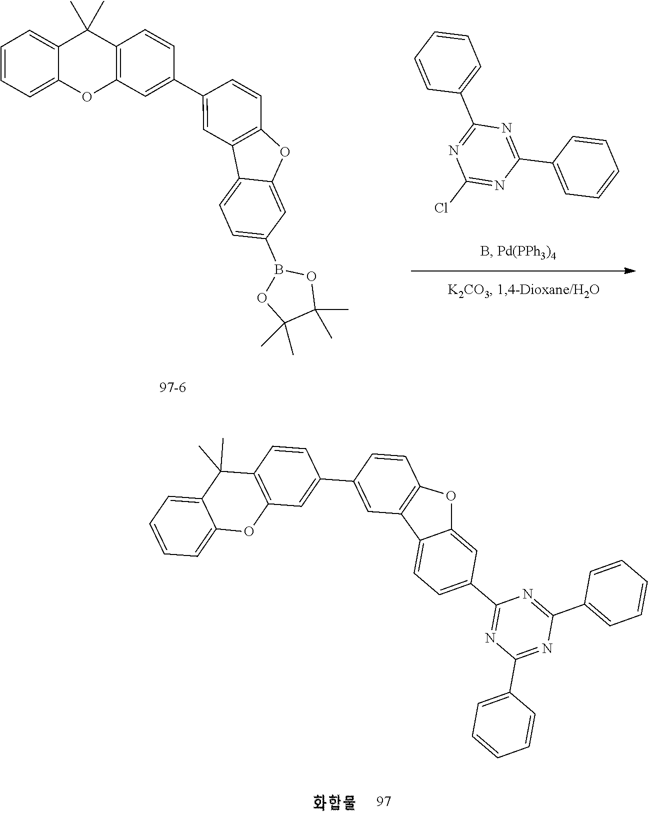 [SMILES: CC1(C)c2ccccc2Oc2cc(-c3ccc4oc5cc(B6OC(C)(C)C(C)(C)O6)ccc5c4c3)ccc21 ; Clc1nc(-c2ccccc2)nc(-c2ccccc2)n1 ; CC1(C)c2ccccc2Oc2cc(-c3ccc4oc5cc(-c6nc(-c7ccccc7)nc(-c7ccccc7)n6)ccc5c4c3)ccc21]

Target Compound 97(H) (12.7 g, 95%) was obtained in the same manner as in Preparation of Compound 1 of Preparation Example 1 except that 2,4-dibromo-1-fluorobenzene was used instead of 1,3-dibromo-2-fluorobenzene.

The following compounds were synthesized in the same manner as in Preparation Example 4 except that Intermediates A and B of the following Table 4 were used instead of using A and B.

TABLE 4

| | | | | Yield |
|---|---|---|---|---|
| | | | | (%) |
| | | | | (97-4 |
| Compound | A | B | C | to C) |
| 133 | 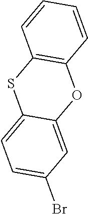 [SMILES: Brc1ccc2c(c1)Oc1ccccc1S2] | 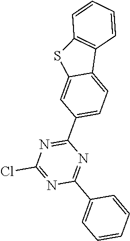 [SMILES: Clc1nc(-c2ccccc2)nc(-c2ccc3c(c2)sc2ccccc23)n1] | 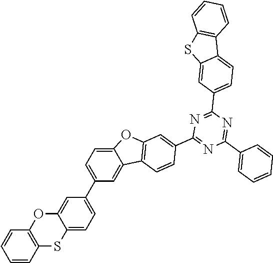 [SMILES: c1ccc(-c2nc(-c3ccc4c(c3)oc3ccc(-c5ccc6c(c5)Oc5ccccc5S6)cc34)nc(-c3ccc4c(c3)sc3ccccc34)n2)cc1] | 78 |
| 134 | 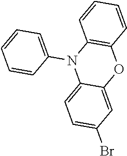 [SMILES: Brc1ccc2c(c1)Oc1ccccc1N2c1ccccc1] | 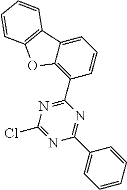 [SMILES: Clc1nc(-c2ccccc2)nc(-c2cccc3c2oc2ccccc23)n1] | 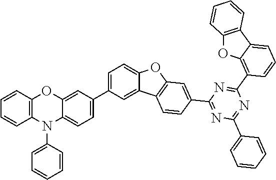 [SMILES: c1ccc(-c2nc(-c3ccc4c(c3)oc3ccc(-c5ccc6c(c5)Oc5ccccc5N6c5ccccc5)cc34)nc(-c3cccc4c3oc3ccccc34)n2)cc1] | 77 |
| 135 | 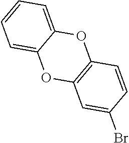 [SMILES: Brc1ccc2c(c1)Oc1ccccc1O2] | 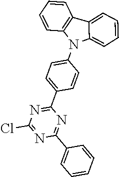 [SMILES: Clc1nc(-c2ccccc2)nc(-c2ccc(-n3c4ccccc4c4ccccc43)cc2)n1] | 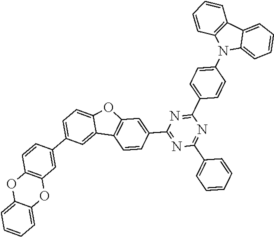 [SMILES: c1ccc(-c2nc(-c3ccc(-n4c5ccccc5c5ccccc54)cc3)nc(-c3ccc4c(c3)oc3ccc(-c5ccc6c(c5)Oc5ccccc5O6)cc34)n2)cc1] | 79 |
| 136 | 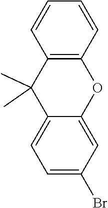 [SMILES: CC1(C)c2ccccc2Oc2cc(Br)ccc21] | 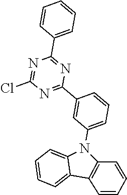 [SMILES: Clc1nc(-c2ccccc2)nc(-c2cccc(-n3c4ccccc4c4ccccc43)c2)n1] | 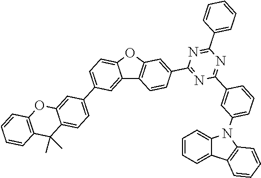 [SMILES: CC1(C)c2ccccc2Oc2cc(-c3ccc4oc5cc(-c6nc(-c7ccccc7)nc(-c7cccc(-n8c9ccccc9c9ccccc98)c7)n6)ccc5c4c3)ccc21] | 82 |
| 137 | 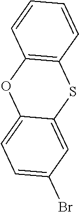 [SMILES: Brc1ccc2c(c1)Sc1ccccc1O2] | 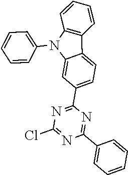 [SMILES: Clc1nc(-c2ccccc2)nc(-c2ccc3c4ccccc4n(-c4ccccc4)c3c2)n1] | 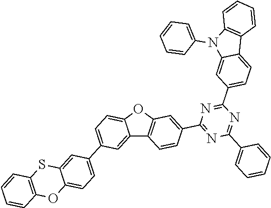 [SMILES: c1ccc(-c2nc(-c3ccc4c(c3)oc3ccc(-c5ccc6c(c5)Sc5ccccc5O6)cc34)nc(-c3ccc4c5ccccc5n(-c5ccccc5)c4c3)n2)cc1] | 83 |
| 138 | 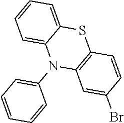 [SMILES: Brc1ccc2c(c1)N(c1ccccc1)c1ccccc1S2] | 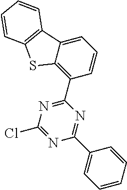 [SMILES: Clc1nc(-c2ccccc2)nc(-c2cccc3c2sc2ccccc23)n1] | 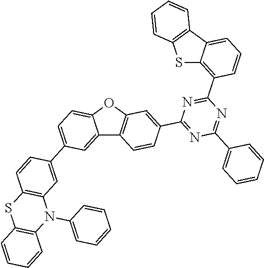 [SMILES: c1ccc(-c2nc(-c3ccc4c(c3)oc3ccc(-c5ccc6c(c5)N(c5ccccc5)c5ccccc5S6)cc34)nc(-c3cccc4c3sc3ccccc34)n2)cc1] | 85 |
| 139 | 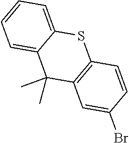 [SMILES: CC1(C)c2ccccc2Sc2ccc(Br)cc21] | 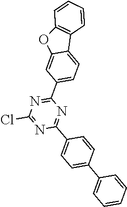 [SMILES: Clc1nc(-c2ccc(-c3ccccc3)cc2)nc(-c2ccc3c(c2)oc2ccccc23)n1] | 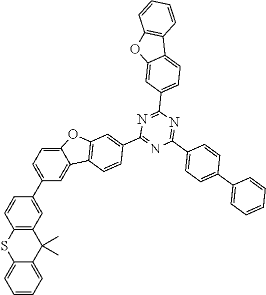 [SMILES: CC1(C)c2ccccc2Sc2ccc(-c3ccc4oc5cc(-c6nc(-c7ccc(-c8ccccc8)cc7)nc(-c7ccc8c(c7)oc7ccccc78)n6)ccc5c4c3)cc21] | 75 |

### [Preparation Example 5] Preparation of Compound 145 (I)

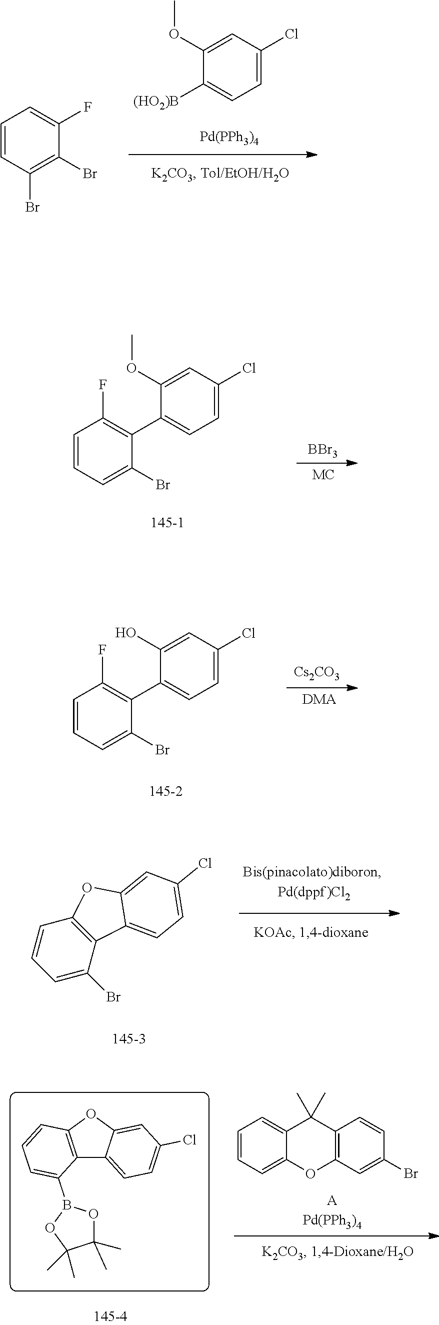

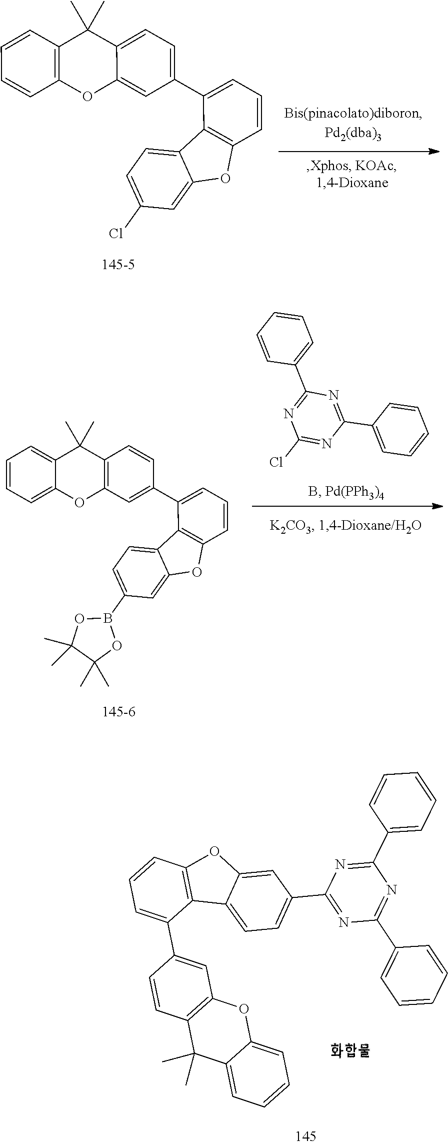 [SMILES: CC1(C)c2ccccc2Oc2cc(-c3cccc4oc5cc(Cl)ccc5c34)ccc21 ; CC1(C)c2ccccc2Oc2cc(-c3cccc4oc5cc(B6OC(C)(C)C(C)(C)O6)ccc5c34)ccc21 ; Clc1nc(-c2ccccc2)nc(-c2ccccc2)n1 ; CC1(C)c2ccccc2Oc2cc(-c3cccc4oc5cc(-c6nc(-c7ccccc7)nc(-c7ccccc7)n6)ccc5c34)ccc21]

Target Compound 145(I) (12.7 g, 95%) was obtained in the same manner as in Preparation of Compound 1 of Preparation Example 1 except that 1,2-dibromo-3-fluorobenzene was used instead of 1,3-dibromo-2-fluorobenzene.

The following compounds were synthesized in the same manner as in Preparation Example 5 except that Intermediates A and B of the following Table 5 were used instead of using A and B.

TABLE 5

| | | | | Yield |
|---|---|---|---|---|
| | | | | (%) |
| | | | | (145-4 |
| Compound | A | B | C | to C) |
| 172 | 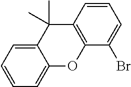 [SMILES: CC1(C)c2ccccc2Oc2c(Br)cccc21] | 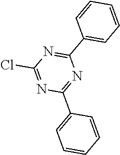 [SMILES: Clc1nc(-c2ccccc2)nc(-c2ccccc2)n1] | 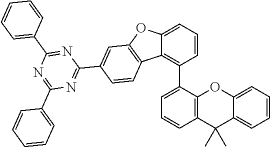 [SMILES: CC1(C)c2ccccc2Oc2c(-c3cccc4oc5cc(-c6nc(-c7ccccc7)nc(-c7ccccc7)n6)ccc5c34)cccc21] | 83 |
| 177 | 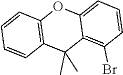 [SMILES: CC1(C)c2ccccc2Oc2cccc(Br)c21] | 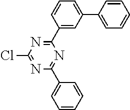 [SMILES: Clc1nc(-c2ccccc2)nc(-c2cccc(-c3ccccc3)c2)n1] | 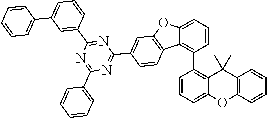 [SMILES: CC1(C)c2ccccc2Oc2cccc(-c3cccc4oc5cc(-c6nc(-c7ccccc7)nc(-c7cccc(-c8ccccc8)c7)n6)ccc5c34)c21] | 88 |
| 178 | 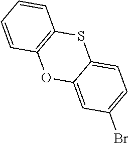 [SMILES: Brc1ccc2c(c1)Oc1ccccc1S2] | 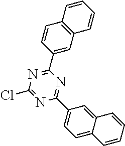 [SMILES: Clc1nc(-c2ccc3ccccc3c2)nc(-c2ccc3ccccc3c2)n1] | 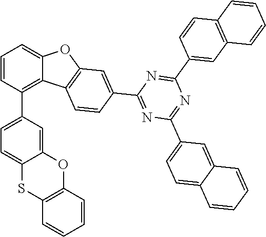 [SMILES: c1ccc2c(c1)Oc1cc(-c3cccc4oc5cc(-c6nc(-c7ccc8ccccc8c7)nc(-c7ccc8ccccc8c7)n6)ccc5c34)ccc1S2] | 88 |
| 179 | 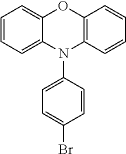 [SMILES: Brc1ccc(N2c3ccccc3Oc3ccccc32)cc1] | 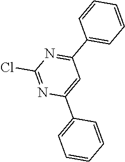 [SMILES: Clc1nc(-c2ccccc2)cc(-c2ccccc2)n1] | 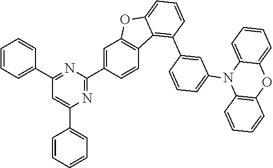 [SMILES: c1ccc(-c2cc(-c3ccccc3)nc(-c3ccc4c(c3)oc3cccc(-c5cccc(N6c7ccccc7Oc7ccccc76)c5)c34)n2)cc1] | 82 |
| 180 | 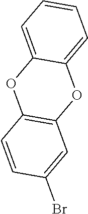 [SMILES: Brc1ccc2c(c1)Oc1ccccc1O2] | 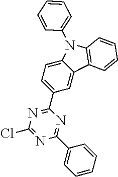 [SMILES: Clc1nc(-c2ccccc2)nc(-c2ccc3c(c2)c2ccccc2n3-c2ccccc2)n1] | 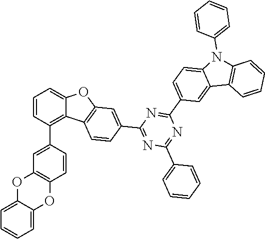 [SMILES: c1ccc(-c2nc(-c3ccc4c(c3)oc3cccc(-c5ccc6c(c5)Oc5ccccc5O6)c34)nc(-c3ccc4c(c3)c3ccccc3n4-c3ccccc3)n2)cc1] | 75 |
| 181 | 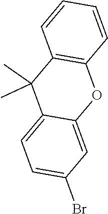 [SMILES: CC1(C)c2ccccc2Oc2cc(Br)ccc21] | 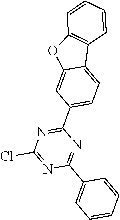 [SMILES: Clc1nc(-c2ccccc2)nc(-c2ccc3c(c2)oc2ccccc23)n1] | 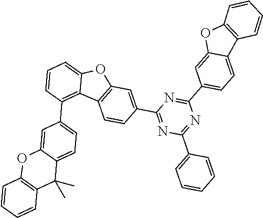 [SMILES: CC1(C)c2ccccc2Oc2cc(-c3cccc4oc5cc(-c6nc(-c7ccccc7)nc(-c7ccc8c(c7)oc7ccccc78)n6)ccc5c34)ccc21] | 77 |

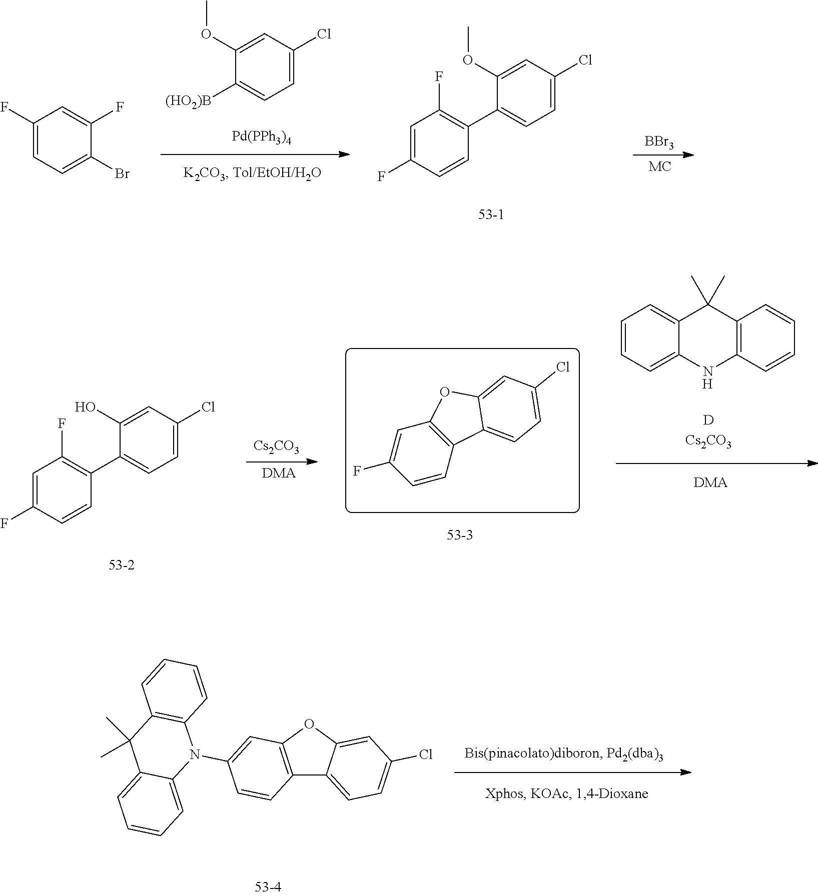

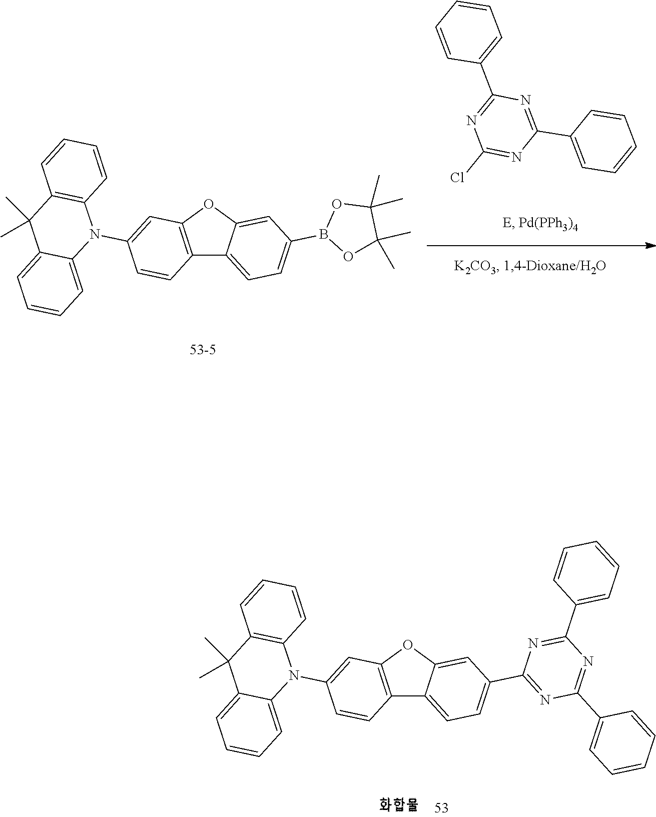

Target Compound 53 (J) was obtained in the same manner as in Preparation Example 2 except that 1-bromo-2,4-difluorobenzene was used instead of 1-bromo-2,3-difluorobenzene.

The following compounds were synthesized in the same manner as in Preparation Example 6 except that Intermediates D and E of the following Table 6 were used instead of using D and E.

TABLE 6

| | | | | Yield |
|---|---|---|---|---|
| | | | | (%) |
| | | | | (53-3 |
| Compound | D | E | F | to F) |
| 54 | 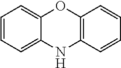 [SMILES: c1ccc2c(c1)Nc1ccccc1O2] | 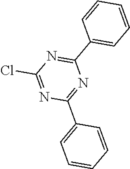 [SMILES: Clc1nc(-c2ccccc2)nc(-c2ccccc2)n1] | 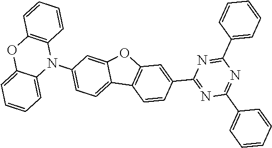 [SMILES: c1ccc(-c2nc(-c3ccccc3)nc(-c3ccc4c(c3)oc3cc(N5c6ccccc6Oc6ccccc65)ccc34)n2)cc1] | 75 |
| 75 | 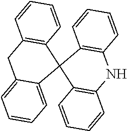 [SMILES: c1ccc2c(c1)Cc1ccccc1C21c2ccccc2Nc2ccccc21] | 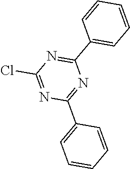 [SMILES: Clc1nc(-c2ccccc2)nc(-c2ccccc2)n1] | 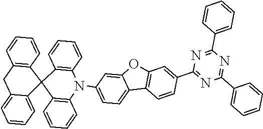 [SMILES: c1ccc(-c2nc(-c3ccccc3)nc(-c3ccc4c(c3)oc3cc(N5c6ccccc6C6(c7ccccc7Cc7ccccc76)c6ccccc65)ccc34)n2)cc1] | 86 |
| 96 | 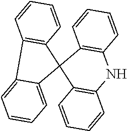 [SMILES: c1ccc2c(c1)Nc1ccccc1C21c2ccccc2-c2ccccc21] | 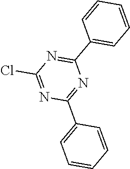 [SMILES: Clc1nc(-c2ccccc2)nc(-c2ccccc2)n1] | 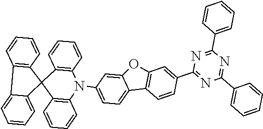 [SMILES: c1ccc(-c2nc(-c3ccccc3)nc(-c3ccc4c(c3)oc3cc(N5c6ccccc6C6(c7ccccc7-c7ccccc76)c6ccccc65)ccc34)n2)cc1] | 73 |

### [Preparation Example 7] Preparation of Compound 101(K)

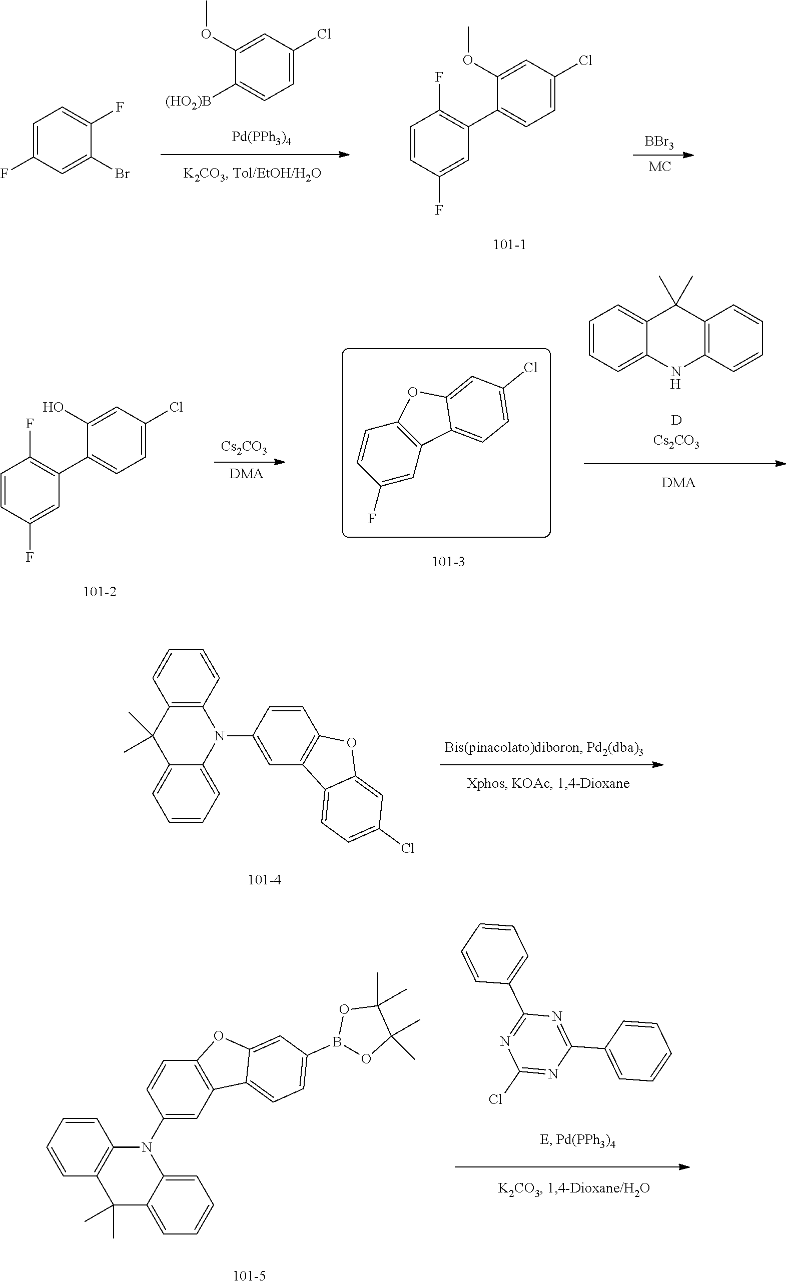

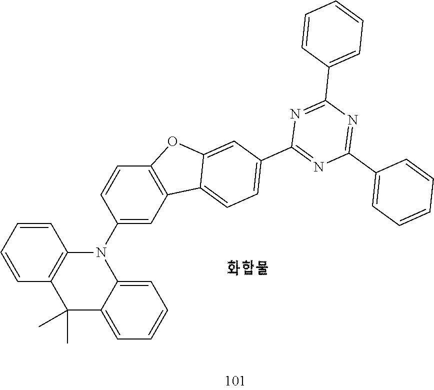 [SMILES: CC1(C)c2ccccc2N(c2ccc3oc4cc(-c5nc(-c6ccccc6)nc(-c6ccccc6)n5)ccc4c3c2)c2ccccc21]

Target Compound 101 (K) was obtained in the same manner as in Preparation Example 2 except that 2-bromo-1,4-difluorobenzene was used instead of 1-bromo-2,3-difluorobenzene.

The following compounds were synthesized in the same manner as in Preparation Example 7 except that Intermediates D and E of the following Table 7 were used instead of using D and E.

TABLE 7

| | | | | Yield |
|---|---|---|---|---|
| | | | | (%) |
| | | | | (101-3 |
| Compound | D | E | F | to F) |
| 103 | 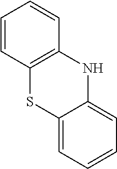 [SMILES: c1ccc2c(c1)Nc1ccccc1S2] | 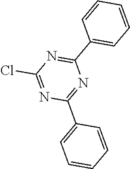 [SMILES: Clc1nc(-c2ccccc2)nc(-c2ccccc2)n1] | 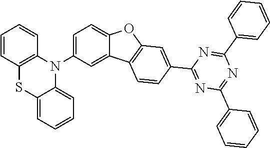 [SMILES: c1ccc(-c2nc(-c3ccccc3)nc(-c3ccc4c(c3)oc3ccc(N5c6ccccc6Sc6ccccc65)cc34)n2)cc1] | 75 |
| 123 | 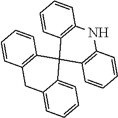 [SMILES: c1ccc2c(c1)Cc1ccccc1C21c2ccccc2Nc2ccccc21] | 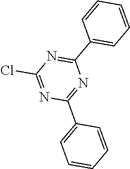 [SMILES: Clc1nc(-c2ccccc2)nc(-c2ccccc2)n1] | 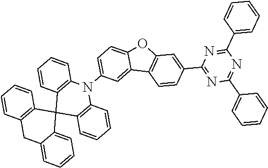 [SMILES: c1ccc(-c2nc(-c3ccccc3)nc(-c3ccc4c(c3)oc3ccc(N5c6ccccc6C6(c7ccccc7Cc7ccccc76)c6ccccc65)cc34)n2)cc1] | 86 |
| 128 | 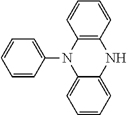 [SMILES: c1ccc(N2c3ccccc3Nc3ccccc32)cc1] | 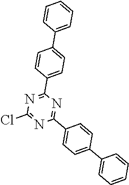 [SMILES: Clc1nc(-c2ccc(-c3ccccc3)cc2)nc(-c2ccc(-c3ccccc3)cc2)n1] | 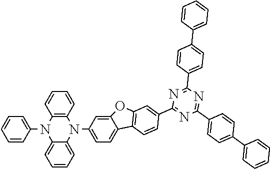 [SMILES: c1ccc(-c2ccc(-c3nc(-c4ccc(-c5ccccc5)cc4)nc(-c4ccc5c(c4)oc4cc(N6c7ccccc7N(c7ccccc7)c7ccccc76)ccc45)n3)cc2)cc1] | 73 |

### [Preparation Example 8] Preparation of Compound 149(L)

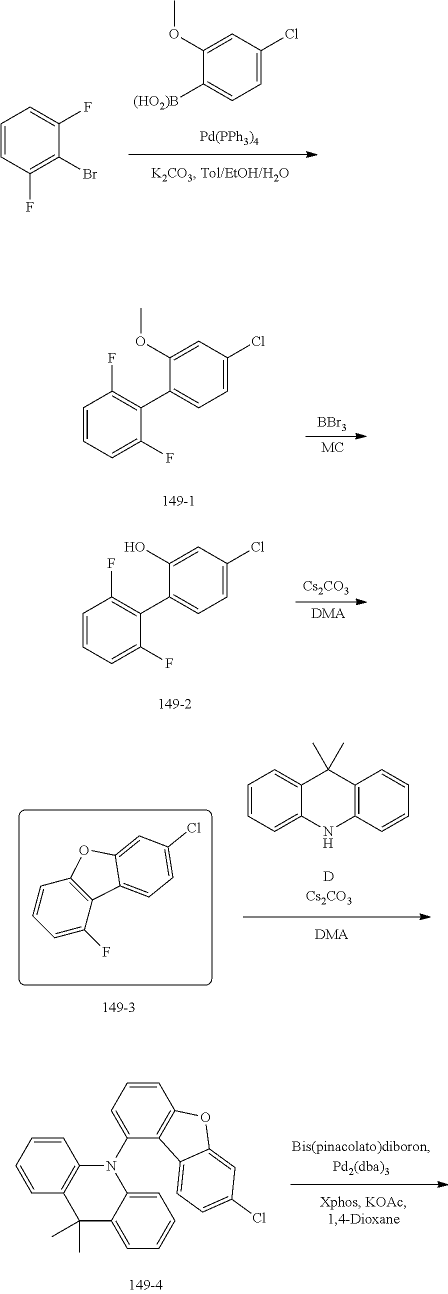

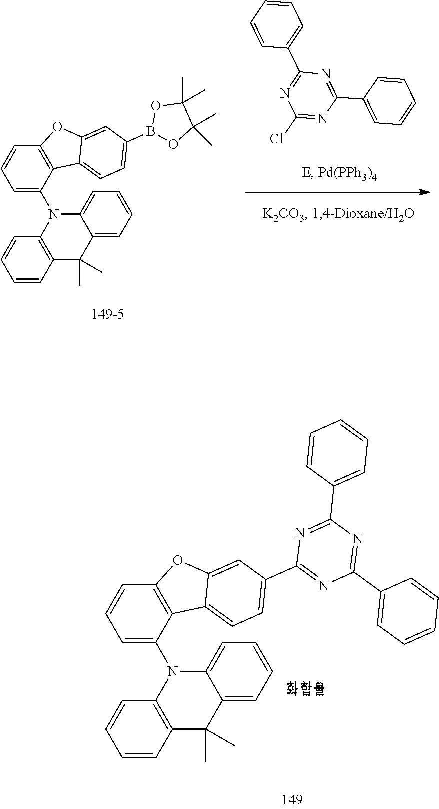 [SMILES: CC1(C)c2ccccc2N(c2cccc3oc4cc(B5OC(C)(C)C(C)(C)O5)ccc4c23)c2ccccc21 ; Clc1nc(-c2ccccc2)nc(-c2ccccc2)n1 ; CC1(C)c2ccccc2N(c2cccc3oc4cc(-c5nc(-c6ccccc6)nc(-c6ccccc6)n5)ccc4c23)c2ccccc21]

Target Compound 149(L) was obtained in the same manner as in Preparation Example 2 except that 2-bromo-1,3-difluorobenzene was used instead of 1-bromo-2,3-difluorobenzene.

The following compounds were synthesized in the same manner as in Preparation Example 8 except that Intermediates D and E of the following Table 8 were used instead of using D and E.

TABLE 8

| | | | | Yield |
|---|---|---|---|---|
| | | | | (%) |
| | | | | (5-3 |
| Compound | D | E | F | to F) |
| 150 | 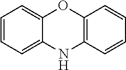 [SMILES: c1ccc2c(c1)Nc1ccccc1O2] | 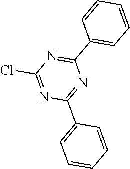 [SMILES: Clc1nc(-c2ccccc2)nc(-c2ccccc2)n1] | 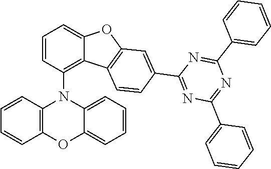 [SMILES: c1ccc(-c2nc(-c3ccccc3)nc(-c3ccc4c(c3)oc3cccc(N5c6ccccc6Oc6ccccc65)c34)n2)cc1] | 75 |
| 151 | 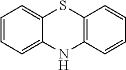 [SMILES: c1ccc2c(c1)Nc1ccccc1S2] | 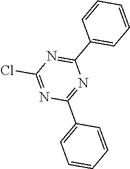 [SMILES: Clc1nc(-c2ccccc2)nc(-c2ccccc2)n1] | 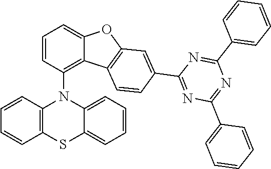 [SMILES: c1ccc(-c2nc(-c3ccccc3)nc(-c3ccc4c(c3)oc3cccc(N5c6ccccc6Sc6ccccc65)c34)n2)cc1] | 86 |
| 152 | 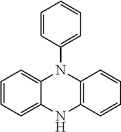 [SMILES: c1ccc(N2c3ccccc3Nc3ccccc32)cc1] | 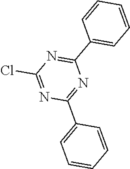 [SMILES: Clc1nc(-c2ccccc2)nc(-c2ccccc2)n1] | 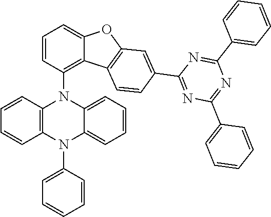 [SMILES: c1ccc(-c2nc(-c3ccccc3)nc(-c3ccc4c(c3)oc3cccc(N5c6ccccc6N(c6ccccc6)c6ccccc65)c34)n2)cc1] | 73 |

Compounds 1 to 200 other than the compounds described in Tables 1 to 8 were also prepared using the same methods described in the preparation examples described above.

The synthesis identification data for the compounds prepared above are as described in the following [Table 9] and [Table 10].

TABLE 9

| Compound | FD-Mass | Compound | FD-Mass |
|---|---|---|---|
| 1 | m/z = 607.70 (C42H29N3O2 = 607.23) | 2 | m/z = 597.68 (C39H23N3O2S = 597.15) |
| 3 | m/z = 656.73 (C45H28N4O2 = 656.22) | 4 | m/z = 581.62 (C39H23N3O3 = 581.17) |
| 5 | m/z = 606.71 (C42H30N4O = 606.24) | 6 | m/z = 580.63 (C39H24N4O2 = 580.19) |
| 7 | m/z = 596.70 (C39H24N4OS = 596.17) | 8 | m/z = 655.74 (C45H29N5O = 655.24) |
| 9 | m/z = 682.81 (C48H34N4O = 682.27) | 10 | m/z = 731.84 (C51H33N5O = 731.27) |
| 11 | m/z = 665.93 (C43H35N3OSi2 = 665.23) | 12 | m/z = 633.78 (C45H35N3O = 633.28) |
| 13 | m/z = 649.85 (C44H35N3OSi = 649.25) | 14 | m/z = 623.77 (C41H29N3O2Si = 623.20) |
| 15 | m/z = 639.84 (C41H29N3OSSi = 639.18) | 16 | m/z = 698.88 (C47H34N4OSi = 698.25) |
| 17 | m/z = 607.70 (C42H29N3O2 = 607.23) | 18 | m/z = 597.68 (C39H23N3O2S = 597.15) |
| 19 | m/z = 656.73 (C45H28N4O2 = 656.22) | 20 | m/z = 581.62 (C39H23N3O3 = 581.17) |
| 21 | m/z = 607.70 (C42H29N3O2 = 607.23) | 22 | m/z = 597.68 (C39H23N3O2S = 597.15) |
| 23 | m/z = 672.80 (C45H28N4OS = 672.20) | 24 | m/z = 623.76 (C42H29N3OS = 623.20) |
| 25 | m/z = 729.82 (C52H31N3O2 = 729.24) | 26 | m/z = 729.82 (C52H31N3O2 = 729.24) |
| 27 | m/z = 742.86 (C53H34N4O = 742.27) | 28 | m/z = 607.70 (C42H29N3O2 = 607.23) |
| 29 | m/z = 682.81 (C48H34N4O = 682.27) | 30 | m/z = 656.73 (C45H28N4O2 = 656.22) |
| 31 | m/z = 672.80 (C45H28N4OS = 672.20) | 32 | m/z = 807.94 (C57H37N5O = 807.30) |
| 33 | m/z = 683.79 (C51H3ON4OS = 683.26) | 34 | m/z = 697.80 (C47H27N3O2S = 697.18) |
| 35 | m/z = 655.74 (C46H29N3O2 = 655.23) | 36 | m/z = 746.81 (C51H30N4O3 = 746.23) |
| 37 | m/z = 697.78 (C48H31N3O3 = 697.24) | 38 | m/z = 703.83 (C45H25N3O2S2 = 703.14) |
| 39 | m/z = 746.81 (C51H30N4O3 = 746.23) | 40 | m/z = 746.81 (C51H30N4O3 = 746.23) |
| 41 | m/z = 772.89 (C54H36N4O2 = 772.28) | 42 | m/z = 762.87 (C51H30N4O2S = 762.21) |
| 43 | m/z = 778.94 (C51H30N4OS2 = 778.19) | 44 | m/z = 789.94 (C54H35N3O2S = 789.24) |
| 45 | m/z = 773.87 (C54H35N3O3 = 773.27) | 46 | m/z = 805.92 (C58H35N3O2 = 805.27) |
| 47 | m/z = 818.96 (C59H38N4O = 818.30) | 48 | m/z = 805.92 (C58H35N3O2 = 805.27) |
| 49 | m/z = 607.70 (C42H29N3O2 = 607.23) | 50 | m/z = 597.68 (C39H23N3O2S = 597.15) |
| 51 | m/z = 656.73 (C45H28N4O2 = 656.22) | 52 | m/z = 581.62 (C39H23N3O3 = 581.17) |
| 53 | m/z = 606.71 (C42H30N4O = 606.24) | 54 | m/z = 580.63 (C39H24N4O2 = 580.19) |
| 55 | m/z = 596.70 (C39H24N4OS = 596.17) | 56 | m/z = 655.74 (C45H29N5O = 655.24) |
| 57 | m/z = 682.81 (C48H34N4O = 682.27) | 58 | m/z = 731.84 (C51H33N5O = 731.27) |
| 59 | m/z = 665.93 (C43H35N3OSi2 = 665.23) | 60 | m/z = 633.78 (C45H35N3O = 633.28) |
| 61 | m/z = 649.85 (C44H35N3OSi = 649.25) | 62 | m/z = 623.77 (C41H29N3O2Si = 623.20) |
| 63 | m/z = 639.84 (C41H29N3OSSi = 639.18) | 64 | m/z = 698.88 (C47H34N4OSi = 698.25) |
| 65 | m/z = 607.70 (C42H29N3O2 = 607.23) | 66 | m/z = 597.68 (C39H23N3O2S = 597.15) |
| 67 | m/z = 656.73 (C45H28N4O2 = 656.22) | 68 | m/z = 581.62 (C39H23N3O3 = 581.17) |
| 69 | m/z = 607.70 (C42H29N3O2 = 607.23) | 70 | m/z = 597.68 (C39H23N3O2S = 597.15) |
| 71 | m/z = 672.80 (C45H28N4OS = 672.20) | 72 | m/z = 623.76 (C42H29N3OS = 623.20) |
| 73 | m/z = 729.82 (C52H31N3O2 = 729.24) | 74 | m/z = 729.82 (C52H31N3O2 = 729.24) |
| 75 | m/z = 742.86 (C53H34N4O = 742.27) | 76 | m/z = 607.70 (C42H29N3O2 = 607.23) |
| 77 | m/z = 682.81 (C48H34N4O = 682.27) | 78 | m/z = 656.73 (C45H28N4O2 = 656.22) |
| 79 | m/z = 672.80 (C45H28N4OS = 672.20) | 80 | m/z = 807.94 (C57H37N5O = 807.30) |
| 81 | m/z = 683.79 (C51H3ON4OS = 683.26) | 82 | m/z = 697.80 (C47H27N3O2S = 697.18) |
| 83 | m/z = 655.74 (C46H29N3O2 = 655.23) | 84 | m/z = 746.81 (C51H30N4O3 = 746.23) |
| 85 | m/z = 697.78 (C48H31N3O3 = 697.24) | 86 | m/z = 703.83 (C45H25N3O2S2 = 703.14) |
| 87 | m/z = 746.81 (C51H30N4O3 = 746.23) | 88 | m/z = 746.81 (C51H30N4O3 = 746.23) |
| 89 | m/z = 772.89 (C54H36N4O2 = 772.28) | 90 | m/z = 762.87 (C51H30N4O2S = 762.21) |
| 91 | m/z = 778.94 (C51H30N4OS2 = 778.19) | 92 | m/z = 789.94 (C54H35N3O2S = 789.24) |
| 93 | m/z = 773.87 (C54H35N3O3 = 773.27) | 94 | m/z = 805.92 (C58H35N3O2 = 805.27) |
| 95 | m/z = 818.96 (C59H38N4O = 818.30) | 96 | m/z = 805.92 (C58H35N3O2 = 805.27) |
| 97 | m/z = 607.70 (C42H29N3O2 = 607.23) | 98 | m/z = 597.68 (C39H23N3O2S = 597.15) |
| 99 | m/z = 656.73 (C45H28N4O2 = 656.22) | 100 | m/z = 581.62 (C39H23N3O3 = 581.17) |
| 101 | m/z = 606.71 (C42H30N4O = 606.24) | 102 | m/z = 580.63 (C39H24N4O2 = 580.19) |
| 103 | m/z = 596.70 (C39H24N4OS = 596.17) | 104 | m/z = 655.74 (C45H29N5O = 655.24) |
| 105 | m/z = 682.81 (C48H34N4O = 682.27) | 106 | m/z = 731.84 (C51H33N5O = 731.27) |
| 107 | m/z = 665.93 (C43H35N3OSi2 = 665.23) | 108 | m/z = 633.78 (C45H35N3O = 633.28) |
| 109 | m/z = 649.85 (C44H35N3OSi = 649.25) | 110 | m/z = 623.77 (C41H29N3O2Si = 623.20) |
| 111 | m/z = 639.84 (C41H29N3OSSi = 639.18) | 112 | m/z = 698.88 (C47H34N4OSi = 698.25) |
| 113 | m/z = 607.70 (C42H29N3O2 = 607.23) | 114 | m/z = 597.68 (C39H23N3O2S = 597.15) |
| 115 | m/z = 656.73 (C45H28N4O2 = 656.22) | 116 | m/z = 581.62 (C39H23N3O3 = 581.17) |
| 117 | m/z = 607.70 (C42H29N3O2 = 607.23) | 118 | m/z = 597.68 (C39H23N3O2S = 597.15) |
| 119 | m/z = 672.80 (C45H28N4OS = 672.20) | 120 | m/z = 623.76 (C42H29N3OS = 623.20) |
| 121 | m/z = 729.82 (C52H31N3O2 = 729.24) | 122 | m/z = 729.82 (C52H31N3O2 = 729.24) |
| 123 | m/z = 742.86 (C53H34N4O = 742.27) | 124 | m/z = 607.70 (C42H29N3O2 = 607.23) |
| 125 | m/z = 682.81 (C48H34N4O = 682.27) | 126 | m/z = 656.73 (C45H28N4O2 = 656.22) |
| 127 | m/z = 672.80 (C45H28N4OS = 672.20) | 128 | m/z = 807.94 (C57H37N5O = 807.30) |
| 129 | m/z = 683.79 (C51H3ON4OS = 683.26) | 130 | m/z = 697.80 (C47H27N3O2S = 697.18) |
| 131 | m/z = 655.74 (C46H29N3O2 = 655.23) | 132 | m/z = 746.81 (C51H30N4O3 = 746.23) |
| 133 | m/z = 697.78 (C48H31N3O3 = 697.24) | 134 | m/z = 703.83 (C45H25N3O2S2 = 703.14) |
| 135 | m/z = 746.81 (C51H30N4O3 = 746.23) | 136 | m/z = 746.81 (C51H30N4O3 = 746.23) |
| 137 | m/z = 772.89 (C54H36N4O2 = 772.28) | 138 | m/z = 762.87 (C51H30N4O2S = 762.21) |
| 139 | m/z = 778.94 (C51H30N4OS2 = 778.19) | 140 | m/z = 789.94 (C54H35N3O2S = 789.24) |
| 141 | m/z = 773.87 (C54H35N3O3 = 773.27) | 142 | m/z = 805.92 (C58H35N3O2 = 805.27) |
| 143 | m/z = 818.96 (C59H38N4O = 818.30) | 144 | m/z = 805.92 (C58H35N3O2 = 805.27) |
| 145 | m/z = 607.70 (C42H29N3O2 = 607.23) | 146 | m/z = 597.68 (C39H23N3O2S = 597.15) |
| 147 | m/z = 656.73 (C45H28N4O2 = 656.22) | 148 | m/z = 581.62 (C39H23N3O3 = 581.17) |
| 149 | m/z = 606.71 (C42H30N4O = 606.24) | 150 | m/z = 580.63 (C39H24N4O2 = 580.19) |
| 151 | m/z = 596.70 (C39H24N4OS = 596.17) | 152 | m/z = 655.74 (C45H29N5O = 655.24) |
| 153 | m/z = 682.81 (C48H34N4O = 682.27) | 154 | m/z = 731.84 (C51H33N5O = 731.27) |
| 155 | m/z = 665.93 (C43H35N3OSi2 = 665.23) | 156 | m/z = 633.78 (C45H35N3O = 633.28) |
| 157 | m/z = 649.85 (C44H35N3OSi = 649.25) | 158 | m/z = 623.77 (C41H29N3O2Si = 623.20) |
| 159 | m/z = 639.84 (C41H29N3OSSi = 639.18) | 160 | m/z = 698.88 (C47H34N4OSi = 698.25) |
| 161 | m/z = 607.70 (C42H29N3O2 = 607.23) | 162 | m/z = 597.68 (C39H23N3O2S = 597.15) |
| 163 | m/z = 656.73 (C45H28N4O2 = 656.22) | 164 | m/z = 581.62 (C39H23N3O3 = 581.17) |
| 165 | m/z = 607.70 (C42H29N3O2 = 607.23) | 166 | m/z = 597.68 (C39H23N3O2S = 597.15) |
| 167 | m/z = 672.80 (C45H28N4OS = 672.20) | 168 | m/z = 623.76 (C42H29N3OS = 623.20) |
| 169 | m/z = 729.82 (C52H31N3O2 = 729.24) | 170 | m/z = 729.82 (C52H31N3O2 = 729.24) |
| 171 | m/z = 742.86 (C53H34N4O = 742.27) | 172 | m/z = 607.70 (C42H29N3O2 = 607.23) |
| 173 | m/z = 682.81 (C48H34N4O = 682.27) | 174 | m/z = 656.73 (C45H28N4O2 = 656.22) |
| 175 | m/z = 672.80 (C45H28N4OS = 672.20) | 176 | m/z = 807.94 (C57H37N5O = 807.30) |
| 177 | m/z = 683.79 (C51H3ON4OS = 683.26) | 178 | m/z = 697.80 (C47H27N3O2S = 697.18) |
| 179 | m/z = 655.74 (C46H29N3O2 = 655.23) | 180 | m/z = 746.81 (C51H30N4O3 = 746.23) |
| 181 | m/z = 697.78 (C48H31N3O3 = 697.24) | 182 | m/z = 703.83 (C45H25N3O2S2 = 703.14) |
| 183 | m/z = 746.81 (C51H30N4O3 = 746.23) | 184 | m/z = 746.81 (C51H30N4O3 = 746.23) |
| 185 | m/z = 772.89 (C54H36N4O2 = 772.28) | 186 | m/z = 762.87 (C51H30N4O2S = 762.21) |
| 187 | m/z = 778.94 (C51H30N4OS2 = 778.19) | 188 | m/z = 789.94 (C54H35N3O2S = 789.24) |
| 189 | m/z = 773.87 (C54H35N3O3 = 773.27) | 190 | m/z = 805.92 (C58H35N3O2 = 805.27) |
| 191 | m/z = 818.96 (C59H38N4O = 818.30) | 192 | m/z = 805.92 (C58H35N3O2 = 805.27) |
| 193 | m/z = 729.82 (C52H31N3O2 = 729.82) | 194 | m/z = 805.92 (C58H35N3O2 = 805.27_ |
| 196 | m/z = 729.82 (C52H31N3O2 = 729.82) | 197 | m/z = 819.90 (C58H33N3O3 = 819.25) |
| 198 | m/z = 729.82(C52H31N3O2 = 729.24) | 199 | m/z = 728.83 (C53H32N2O2 = 728.25) |
| 199 | m/z = 895.01 (C64H38N4O2 = 894.30) | 200 | m/z = 805.92 (C58H35N3O2 = 805.27) |

TABLE 10

| Compound | ¹H NMR (CDCl₃, 300 Mz ) |
|---|---|
| 1 | δ = 8.28(m, 4H), 7.95(d, 1H), 7.85-7.75(m, 3H), 7.64(d, 1H), 7.51- |
| | 7.23(m, 12H), 7.09(m, 2H), 1.72 (s, 6H) |
| 7 | δ = 8.28(m, 4H), 7.95(d, 1H), 7.75(d, 1H), 7.64(s, 1H), 7.51-7.41(m, |
| | 6H), 7.25-7.16(m, 7H), 7.07-6.97(m, 3H0, 6.39(m, 1H) |
| 12 | δ = 8.28(m, 4H), 7.95(d, 1H), 7.85-7.75(m, 3H), 7.64(d, 1H), 7.53- |
| | 7.51(m, 5H), 7.41-7.31(m, 7H), 7.19(m, 2H), 1.72(s, 12H) |
| 18 | δ = 8.28(m, 4H), 7.95(d, 1H), 7.85-7.75(m, 3H), 7.64(s, 1H), 7.51- |
| | 7.37(m, 10H), 7.15(m, 1H), 7.00-6.94(m, 3H) |
| 46 | δ = 8.288.24(m, 3H), 7.95(d, 1H), 7.87-7.70(m, 6H), 7.57-7.19(m, |
| | 23H), 7.05(m, 2H) |
| 51 | δ = 8.28(m, 4H), 7.95(d, 2H), 7.75(d, 2H), 7.64(s, 2H), 7.51-7.41(m, |
| | 6H), 7.20(m, 2H), 7.11(m, 1H), 6.95-6.77(m, 6H), 6.63-6.59(m, 3H) |
| 57 | δ = 8.28(m, 4H), 7.95(d, 2H), 7.81-7.73(m, 2H), 7.75(d, 2H), 7.64- |
| | 7.61(m, 3H), 7.51-7.36(m, 7H), 7.20(m, 2H), 7.05-7.04(m, 2H), 7.03- |
| | 6.55(m, 4H), 1.72(s, 6H) |
| 68 | δ = 8.28(m, 4H), 7.95(d, 2H), 7.75(m, 2H), 7.64(s, 2H), 7.51-7.41(m, |
| | 8H), 7.16-7.13(m, 3H), 6.82(m, 2H) |
| 86 | δ = 8.45(d, 1H), 8.28(m, 2H), 8.11-8.08(m, 2H), 7.98-7.95(m, 3H), |
| | 7.82-7.75 (m, 3H), 7.64 (s, 2H), 7.51-7.31 (m, 9H), 7.15 (m, 1H), 6.97- |
| | 6.94(m, 2H) |
| 93 | δ = 8.24(m, 1H), 7.95-7.89(m, 4H), 7.75-7.23(m, 22H), 7.09(m, 2H), |
| | 1.72(s, 6H) |
| 101 | δ = 8.28(m, 4H), 7.95(d, 1H), 7.75(d, 1H), 7.65-7.64(m, 2H), 7.51- |
| | 7.41(m, 7H), 7.05-7.02(m, 4H), 6.73(m, 2H), 6.55(m, 2H), 6.39(m, |
| | 1H), 1.72 (s, 6H) |
| 111 | δ = 8.28(m, 4H), 7.95(d, 1H), 7.81-7.71(m, 4H), 7.64-7.63(m, 2H), |
| | 7.51-7.32(m, 1H), 7.11(m, 1H), 0.66(s, 6H) |
| 118 | δ = 8.28(m, 4H), 7.95(d, 1H), 7.81-7.71(m, 4H), 7.64(s, 1H), 7.51- |
| | 7.37(m, 9H), 7.15(m, 1H), 7.00-6.94(m, 3H) |
| 131 | δ = 8.28 (m, 2H), 7.95-7.89 (m, 3H), 7.81-7.64 (m, 8H), 7.51 (m, 2H), |
| | 7.43-7.23(m, 8H), 7.09(m, 2H), 1.72(s, 6H) |
| 143 | δ = 8.28(m, 4H), 7.95(m, 1H), 7.87(m, 2H), 7.75(m, 1H), 7.65-7.64(m, |
| | 2H), 7.51-7.28(m, 13H), 7.01(m, 4H), 6.69(m, 2H), 6.51(m, 2H), |
| | 6.39(m, 1H) |
| 148 | δ = 8.28 (m, 4H), 7.95 (m, 1H), 7.75 (m, 2H), 7.64-7.62 (m, 2H), 7.51- |
| | 7.44(m, 9H), 7.16-7.13(m, 3H), 6.82(d, 2H) |
| 150 | δ = 8.28(m, 4H), 7.95(m, 1H), 7.75(m, 1H), 7.64(s, 1H), 7.51-7.41(m, |
| | 6H), 7.13(m, 1H), 7.02(m, 1H), 6.92-6.89(m, 4H), 6.77(m, 2H), |
| | 6.59(m, 2H), 6.33(m, 1H) |
| 158 | δ = 8.28(m, 4H), 7.95(m, 1H), 7.75(m, 2H), 7.64-7.61(m, 4H), 7.51- |
| | 7.42(m, 10H), 7.24(m, 1H), 7.09(m, 1H), 0.66(s, 6H) |
| 161 | δ = 8.28(m, 4H), 7.95(m, 1H), 7.75(m, 2H), 7.64-7.62(m, 2H), 7.51- |
| | 7.41(m, 8H), 7.32-7.09(m, 4H), 7.08(m, 2H), 1.72(s, 6H) |
| 162 | δ = 8.28(m, 4H), 7.95(d, 1H), 7.75(d, 2H), 7.64-7.62(m, 2H), 7.51- |
| | 7.38(m, 10H), 7.15(m, 1H), 7.00-6.94 (m, 3H) |

### Example

1) Manufacture of Organic Light Emitting Device

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino]triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. The light emitting layer was deposited to 400 Å using a compound described in the following Table 11 as a host, and, as a green phosphorescent dopant, doping Ir(ppy)₃ (tris(2-phenylpyridine)iridium) to the host by 7%. After that, BCP was deposited to 60 Å as a hole blocking layer, and Alq₃ was deposited to 200 Å thereon as an electron transfer layer. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured.

Meanwhile, all the organic compounds required to manufacture the OLED were vacuum sublimation purified under 10⁻⁸ torr to 10⁻⁶ torr for each material to be used in the OLED manufacture.

2) Driving Voltage and Light Emission Efficiency of Organic Electroluminescent Device

For each of the organic electroluminescent devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Properties of the organic electroluminescent devices of the present disclosure are as shown in Table 11.

TABLE 11

| | | Driving | Efficiency | Color Coordinate | Lifetime |
|---|---|---|---|---|---|
| | Compound | Voltage (V) | (cd/A) | (x, y) | (T₉₀) |
| Comparative | A | 5.72 | 46.7 | (0.273, 0.684) | 55 |
| Example 1 | | | | | |
| Comparative | B | 5.55 | 50.2 | (0.277, 0.674) | 65 |
| Example 2 | | | | | |
| Comparative | C | 5.23 | 59.2 | (0.271, 0.686) | 43 |
| Example 3 | | | | | |
| Comparative | D | 5.82 | 47.4 | (0.273, 0.682) | 55 |
| Example 4 | | | | | |
| Comparative | E | 5.99 | 48.7 | (0.278, 0.686 | 43 |
| Example 5 | | | | | |
| Comparative | F | 5.32 | 49.3 | (0.276, 0.683) | 61 |
| Example 6 | | | | | |
| Comparative | G | 5.63 | 49.5 | (0.276, 0.682) | 60 |
| Example 7 | | | | | |
| Example 1 | 1 | 4.01 | 70.2 | (0.277, 0.669) | 174 |
| Example 2 | 2 | 3.62 | 79.3 | (0.281, 0.679) | 169 |
| Example 3 | 3 | 3.66 | 75.2 | (0.280, 0.677) | 168 |
| Example 4 | 4 | 3.98 | 71.7 | (0.279, 0.676) | 154 |
| Example 5 | 9 | 4.64 | 67.2 | (0.272, 0.669) | 138 |
| Example 6 | 10 | 4.32 | 66.4 | (0.271, 0.671) | 140 |
| Example 7 | 11 | 3.88 | 77.2 | (0.275, 0.672) | 158 |
| Example 8 | 12 | 3.74 | 76.3 | (0.279, 0.675) | 162 |
| Example 9 | 13 | 3.84 | 70.4 | (0.280, 0.676) | 170 |
| Example 10 | 14 | 3.72 | 72.6 | (0.278, 0.672) | 169 |
| Example 11 | 15 | 4.53 | 65.4 | (0.283, 0.687) | 134 |
| Example 12 | 16 | 4.32 | 60.2 | (0.286, 0.686) | 138 |
| Example 13 | 17 | 4.42 | 66.4 | (0.274, 0.678) | 142 |
| Example 14 | 18 | 3.99 | 75.2 | (0.279, 0.677) | 169 |
| Example 15 | 19 | 3.88 | 74.2 | (0.278, 0.677) | 160 |
| Example 16 | 20 | 4.02 | 62.3 | (0.276, 0.671) | 152 |
| Example 17 | 21 | 4.46 | 66.4 | (0.284, 0.687) | 149 |
| Example 18 | 22 | 4.51 | 68.4 | (0.281, 0.684) | 153 |
| Example 19 | 23 | 4.21 | 70.2 | (0.283, 0.681) | 146 |
| Example 20 | 24 | 4.11 | 67.5 | (0.279, 0.681) | 146 |
| Example 21 | 25 | 4.08 | 68.7 | (0.280, 0.679) | 132 |
| Example 22 | 26 | 4.32 | 70.2 | (0.281, 0.678) | 137 |
| Example 23 | 28 | 3.86 | 68.2 | (0.278, 0.682) | 141 |
| Example 24 | 33 | 4.01 | 60.3 | (0.286, 0.692) | 132 |
| Example 25 | 34 | 3.85 | 68.2 | (0.274, 0.672) | 138 |
| Example 26 | 35 | 3.71 | 70.4 | (0.278, 0.678) | 139 |
| Example 27 | 36 | 4.11 | 68.4 | (0.277, 0.674) | 151 |
| Example 28 | 37 | 4.32 | 62.2 | (0.279, 0.674) | 143 |
| Example 29 | 38 | 4.23 | 69.3 | (0.278, 0.677) | 153 |
| Example 30 | 39 | 4.31 | 68.2 | (0.283, 0.677) | 141 |
| Example 31 | 40 | 4.18 | 66.4 | (0.273, 0.678) | 149 |
| Example 32 | 41 | 4.06 | 70.1 | (0.277, 0.668) | 158 |
| Example 33 | 42 | 4.02 | 63.4 | (0.271, 0.673) | 132 |
| Example 34 | 43 | 3.99 | 62.9 | (0.276, 0.671) | 138 |
| Example 35 | 44 | 4.12 | 64.2 | (0.272, 0.666) | 164 |
| Example 36 | 45 | 4.33 | 64.2 | (0.274, 0.667) | 124 |
| Example 37 | 46 | 4.23 | 63.4 | (0.276, 0.668) | 133 |
| Example 38 | 5 | 4.43 | 65.2 | (0.275, 0.669) | 139 |
| Example 39 | 6 | 4.58 | 67.3 | (0.275, 0.669) | 141 |
| Example 40 | 7 | 3.62 | 75.3 | (0.274, 0.671) | 170 |
| Example 41 | 8 | 3.85 | 72.3 | (0.274, 0.679) | 169 |
| Example 42 | 27 | 3.75 | 70.4 | (0.276, 0.670) | 171 |
| Example 43 | 29 | 3.93 | 68.3 | (0.280, 0.679) | 162 |
| Example 44 | 30 | 4.32 | 62.9 | (0.289, 0.669) | 142 |
| Example 45 | 31 | 3.89 | 73.4 | (0.276, 0.670) | 182 |
| Example 46 | 32 | 3.60 | 72.9 | (0.280, 0.673) | 173 |
| Example 47 | 47 | 3.63 | 79.3 | (0.281, 0.678) | 169 |
| Example 48 | 48 | 3.86 | 78.4 | (0.280, 0.678) | 166 |
| Example 49 | 49 | 4.72 | 63.4 | (0.278, 0.673) | 142 |
| Example 50 | 50 | 4.23 | 67.2 | (0.276, 0.672) | 139 |
| Example 51 | 51 | 3.90 | 79.9 | (0.277, 0.676) | 148 |
| Example 52 | 52 | 3.77 | 73.4 | (0.280, 0.674) | 167 |
| Example 53 | 57 | 3.92 | 79.7 | (0.281, 0.672) | 178 |
| Example 54 | 58 | 3.73 | 71.3 | (0.280, 0.674) | 178 |
| Example 55 | 59 | 4.33 | 69.1 | (0.282, 0.679) | 142 |
| Example 56 | 97 | 4.42 | 67.5 | (0.284, 0.680) | 149 |
| Example 57 | 133 | 4.21 | 61.9 | (0.277, 0.679) | 138 |
| Example 58 | 134 | 3.79 | 74.8 | (0.278, 0.679) | 172 |
| Example 59 | 135 | 3.99 | 78.3 | (0.279, 0.680) | 166 |
| Example 60 | 136 | 4.12 | 71.3 | (0.279, 0.678) | 171 |
| Example 61 | 137 | 4.32 | 69.7 | (0.282, 0.686) | 153 |
| Example 62 | 138 | 4.41 | 69.2 | (0.283, 0.685) | 159 |
| Example 63 | 139 | 4.29 | 80.4 | (0.284, 0.682) | 159 |
| Example 64 | 145 | 3.99 | 66.3 | (0.280, 0.680) | 141 |
| Example 65 | 172 | 3.91 | 70.4 | (0.285, 0.681) | 130 |
| Example 66 | 178 | 4.02 | 72.8 | (0.280, 0.679) | 121 |
| Example 67 | 179 | 3.88 | 69.4 | (0.277, 0.681) | 132 |
| Example 68 | 180 | 4.22 | 67.8 | (0.281, 0.688) | 148 |
| Example 69 | 181 | 3.79 | 66.6 | (0.277, 0.682) | 141 |
| Example 70 | 53 | 3.87 | 76.2 | (0.279, 0.680) | 149 |
| Example 71 | 54 | 4.19 | 65.2 | (0.279, 0.673) | 158 |
| Example 72 | 75 | 4.03 | 69.4 | (0.280, 0.671) | 155 |
| Example 73 | 96 | 4.24 | 73.3 | (0.276, 0.678) | 165 |
| Example 74 | 101 | 4.11 | 79.3 | (0.282, 0.676) | 152 |
| Example 75 | 103 | 4.32 | 65.2 | (0.276, 0.677) | 129 |
| Example 76 | 123 | 3.87 | 74.8 | (0.278, 0.670) | 138 |
| Example 77 | 128 | 4.23 | 67.7 | (0.279, 0.675) | 149 |
| Example 78 | 149 | 3.91 | 66.9 | (0.278, 0.674) | 171 |
| Example 79 | 150 | 4.23 | 62.3 | (0.277, 0.676) | 177 |
| Example 80 | 151 | 4.13 | 64.8 | (0.278, 0.673) | 132 |
| Example 81 | 152 | 4.42 | 62.9 | (0.280, 0.672) | 141 |

### Comparative Example 1

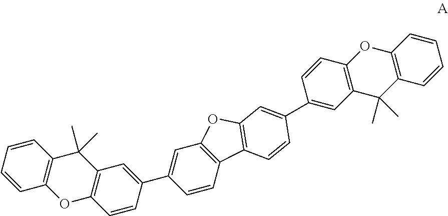 [SMILES: CC1(C)c2ccccc2Oc2ccc(-c3ccc4c(c3)oc3cc(-c5ccc6c(c5)C(C)(C)c5ccccc5O6)ccc34)cc21]

### Comparative Example 2

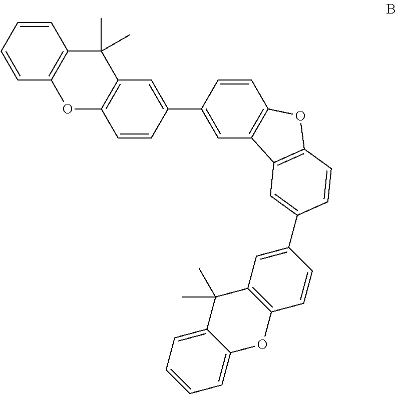 [SMILES: CC1(C)c2ccccc2Oc2ccc(-c3ccc4oc5ccc(-c6ccc7c(c6)C(C)(C)c6ccccc6O7)cc5c4c3)cc21]

### Comparative Example 3

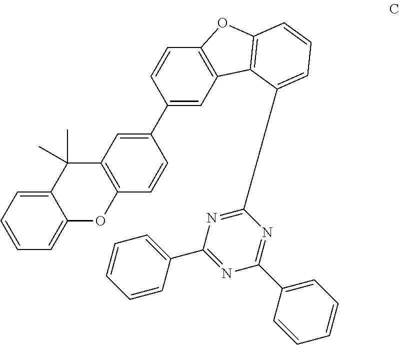 [SMILES: CC1(C)c2ccccc2Oc2ccc(-c3ccc4oc5cccc(-c6nc(-c7ccccc7)nc(-c7ccccc7)n6)c5c4c3)cc21]

### Comparative Example 4

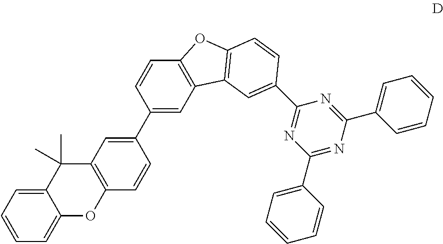 [SMILES: CC1(C)c2ccccc2Oc2ccc(-c3ccc4oc5ccc(-c6nc(-c7ccccc7)nc(-c7ccccc7)n6)cc5c4c3)cc21]

### Comparative Example 5

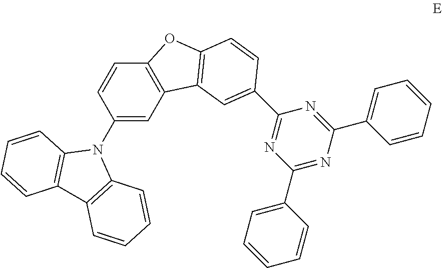 [SMILES: c1ccc(-c2nc(-c3ccccc3)nc(-c3ccc4oc5ccc(-n6c7ccccc7c7ccccc76)cc5c4c3)n2)cc1]

### Comparative Example 6

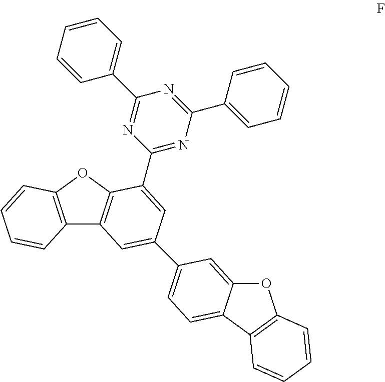 [SMILES: c1ccc(-c2nc(-c3ccccc3)nc(-c3cc(-c4ccc5c(c4)oc4ccccc45)cc4c3oc3ccccc34)n2)cc1]

### Comparative Example 7

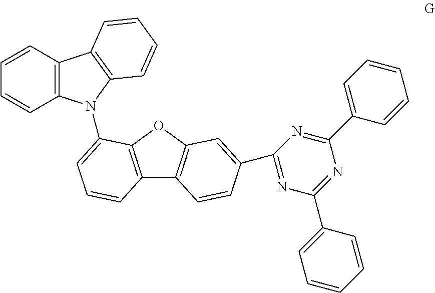 [SMILES: c1ccc(-c2nc(-c3ccccc3)nc(-c3ccc4c(c3)oc3c(-n5c6ccccc6c6ccccc65)cccc34)n2)cc1]

As seen from Table 11, it was identified that, by Ar of Chemical Formula 1 being represented by Chemical Formula 2 or Chemical Formula 3 in the compound according to the present application, electron distribution spread widely from the dibenzofuran core to the substituents of Ar resulting in a wide band gap and a high T1 value. In addition, it was identified that, by Ar of Chemical Formula 1 having the substituents of Chemical Formula 2 or Chemical Formula 3, a stable molecular structure was able to be maintained contributing to enhancement in the lifetime.

Accordingly, it was identified that, due to the properties of wide band gap and high T1 compared to cases of having carbazoles, the organic light emitting device had superior efficiency and low driving voltage in the present application when using Chemical Formula 1 as a phosphorescent host material of the organic light emitting device.

In Table 11, Comparative Example 1 and Comparative Example 2 has a dimethylxanthene group with a symmetric structure as a substituent, and Comparative Examples 3, 4 and 5 relate to compounds having an azine-based substituent, a carbazole group and a xanthene group at various positions of dibenzofuran. Comparative Example 6 of Table 10 has both an azine-based substituent and a dibenzofuran group at position Nos. 2 and 4 of one-side benzene ring of dibenzofuran, and Comparative Example 7 introduces, while having an azine-based substituent at position No. 3 of dibenzofuran, a carbazole group to another benzene ring, and it was identified that the compounds of Comparative Examples 1 to 7 had lower T1 and narrower band gap compared to the compound of Chemical Formula 1 of the present disclosure.

Accordingly, it was seen that Comparative Examples 1 to 7 of Table 11 had properties of high driving voltage and low efficiency compared to when using the compound corresponding to Chemical Formula 1 of the present application.

1. A heterocyclic compound represented by the following Chemical Formula 1: 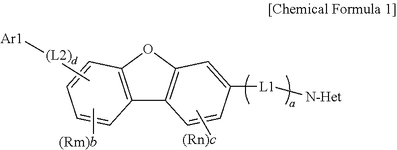
wherein, in Chemical Formula 1,
N-Het is a monocyclic or polycyclic C2 to C60 heterocyclic group substituted or unsubstituted, and comprising one or more Ns;
L1 and L2 are a direct bond; a substituted or unsubstituted C6 to C60 arylene group; or a substituted or unsubstituted C2 to C60 heteroarylene group, a and d are each an integer of 1 to 3, and when a is 2 or greater, L1s are the same as or different from each other, and when d is 2 or greater, L2s are the same as or different from each other;
Rm and Rn are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; and —NRR′, or two or more groups adjacent to each other bond to each other to form a substituted or unsubstituted C6 to C60 aliphatic or aromatic hydrocarbon ring or C2 to C60 heteroring, b and c are each an integer of 1 to 3, and when b is 2 or greater, Rms are the same as or different from each other, and when c is 2 or greater, Rns are the same as or different from each other; and
Ar1 is represented by the following Chemical Formula 2 or 3, 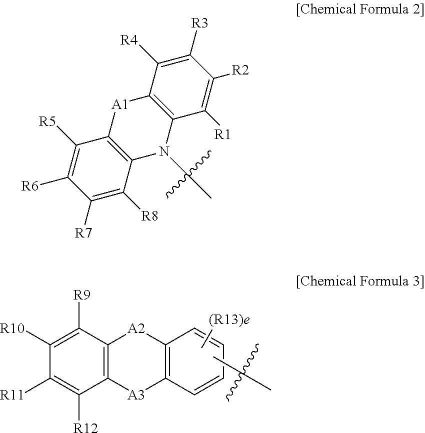
in Chemical Formulae 2 and 3, 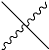
means a site linked to L2 of Chemical Formula 1;
R1 to R13 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; and —NRR′, or two or more groups adjacent to each other bond to each other to form a substituted or unsubstituted C6 to C60 aliphatic or aromatic hydrocarbon ring or C2 to C60 heteroring, e is an integer of 1 to 3, and when e is 2 or greater, R13s are the same as or different from each other;
A1 and A2 are the same as or different from each other, and each independently O; S; CRaRb; NRc; or SiRdRe;
A3 is a direct bond; O; S; CRaRb; NRc; or SiRdRe; and
R, R′ and Ra to Re are the same as or different from each other, and each independently hydrogen; a substituted or unsubstituted C1 to C60 alkyl group; a substituted or unsubstituted C6 to C60 aryl group; or a substituted or unsubstituted C2 to C60 heteroaryl group, or two or more groups adjacent to each other bond to each other to form a substituted or unsubstituted C6 to C60 aliphatic or aromatic hydrocarbon ring or C2 to C60 heteroring.

2. The heterocyclic compound of claim 1, wherein Chemical Formula 1 is represented by any one of the following Chemical Formulae 4 to 7: 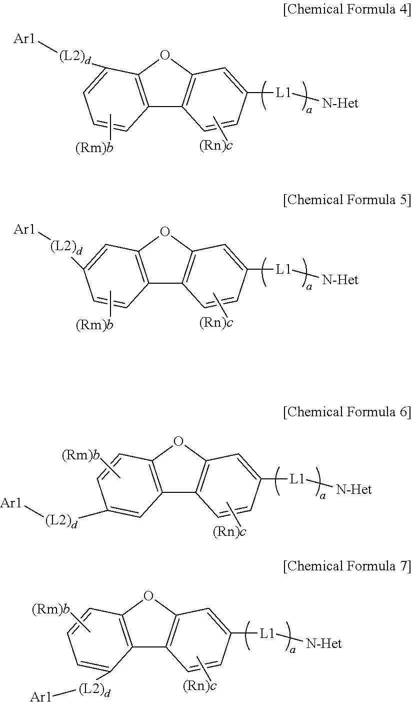
in Chemical Formulae 4 to 7,
each substituent has the same definition as in Chemical Formula 1.

3. The heterocyclic compound of claim 1, wherein N-Het is represented by the following Chemical Formula 8: 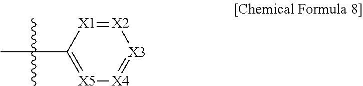
in Chemical Formula 8, 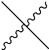
means a site linked to L1 of Chemical Formula 1;
X1 is CR21 or N, X2 is CR22 or N, X3 is CR23 or N, X4 is CR24 or N, and X5 is CR25 or N;
at least one of X1 to X5 is N;
R21 to R25 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′ and —NRR′, or two or more groups adjacent to each other bond to each other to form a substituted or unsubstituted C6 to C60 aliphatic or aromatic hydrocarbon ring or C2 to C60 heteroring; and
R and R′ have the same definitions as in Chemical Formula 1.

4. The heterocyclic compound of claim 3, wherein Chemical Formula 8 is any one selected from among the following structural formulae: 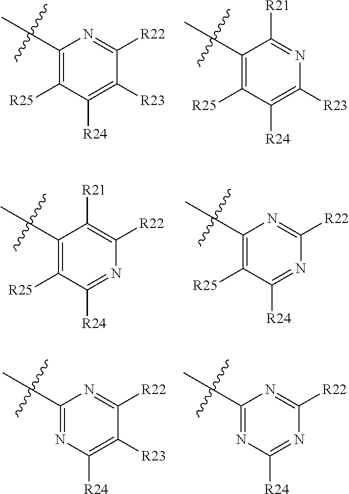
in the structural formulae,
R21 to R25 have the same definitions as in Chemical Formula 8.

5. The heterocyclic compound of claim 1, wherein Chemical Formula 3 is represented by any one of the following Chemical Formulae 3-1 to 3-4: 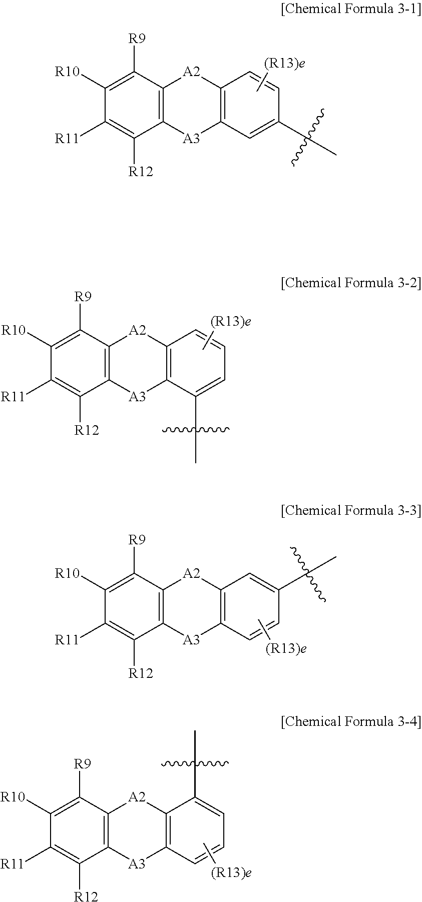
in Chemical Formulae 3-1 to 3-4,
each substituent has the same definition as in Chemical Formula 3.

6. The heterocyclic compound of claim 1, wherein Chemical Formula 1 is represented by any one of the following compounds:

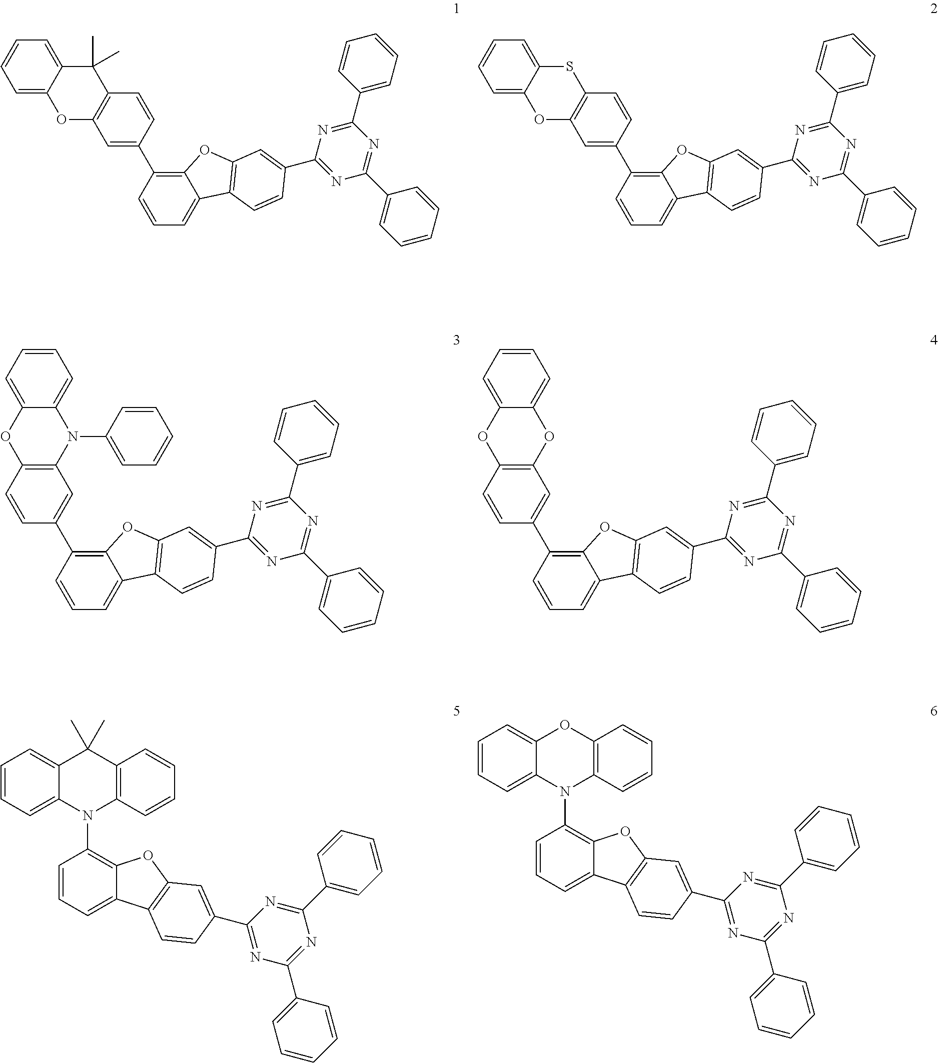 [SMILES: CC1(C)c2ccccc2Oc2cc(-c3cccc4c3oc3cc(-c5nc(-c6ccccc6)nc(-c6ccccc6)n5)ccc34)ccc21 ; c1ccc(-c2nc(-c3ccccc3)nc(-c3ccc4c(c3)oc3c(-c5ccc6c(c5)Oc5ccccc5S6)cccc34)n2)cc1 ; c1ccc(-c2nc(-c3ccccc3)nc(-c3ccc4c(c3)oc3c(-c5ccc6c(c5)N(c5ccccc5)c5ccccc5O6)cccc34)n2)cc1 ; c1ccc(-c2nc(-c3ccccc3)nc(-c3ccc4c(c3)oc3c(-c5ccc6c(c5)Oc5ccccc5O6)cccc34)n2)cc1 ; CC1(C)c2ccccc2N(c2cccc3c2oc2cc(-c4nc(-c5ccccc5)nc(-c5ccccc5)n4)ccc23)c2ccccc21 ; c1ccc(-c2nc(-c3ccccc3)nc(-c3ccc4c(c3)oc3c(N5c6ccccc6Oc6ccccc65)cccc34)n2)cc1]

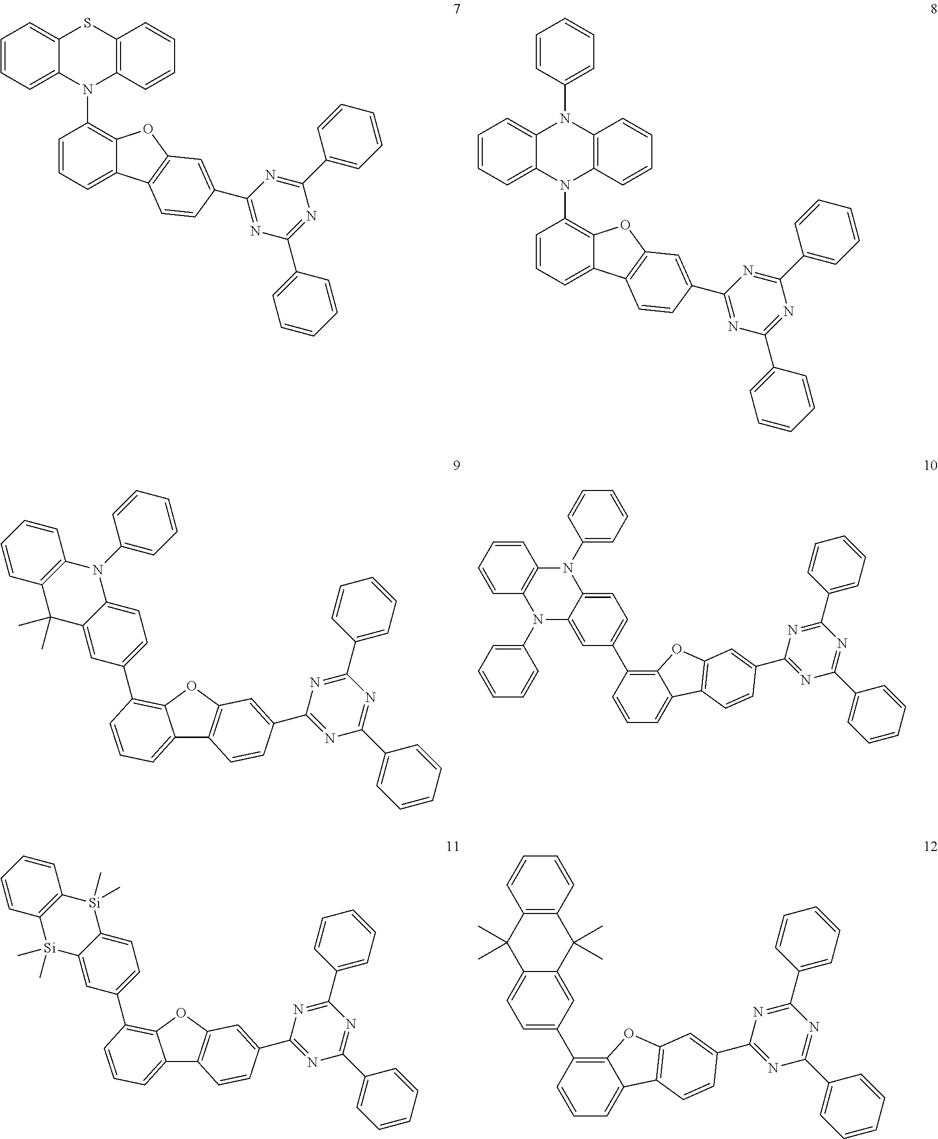

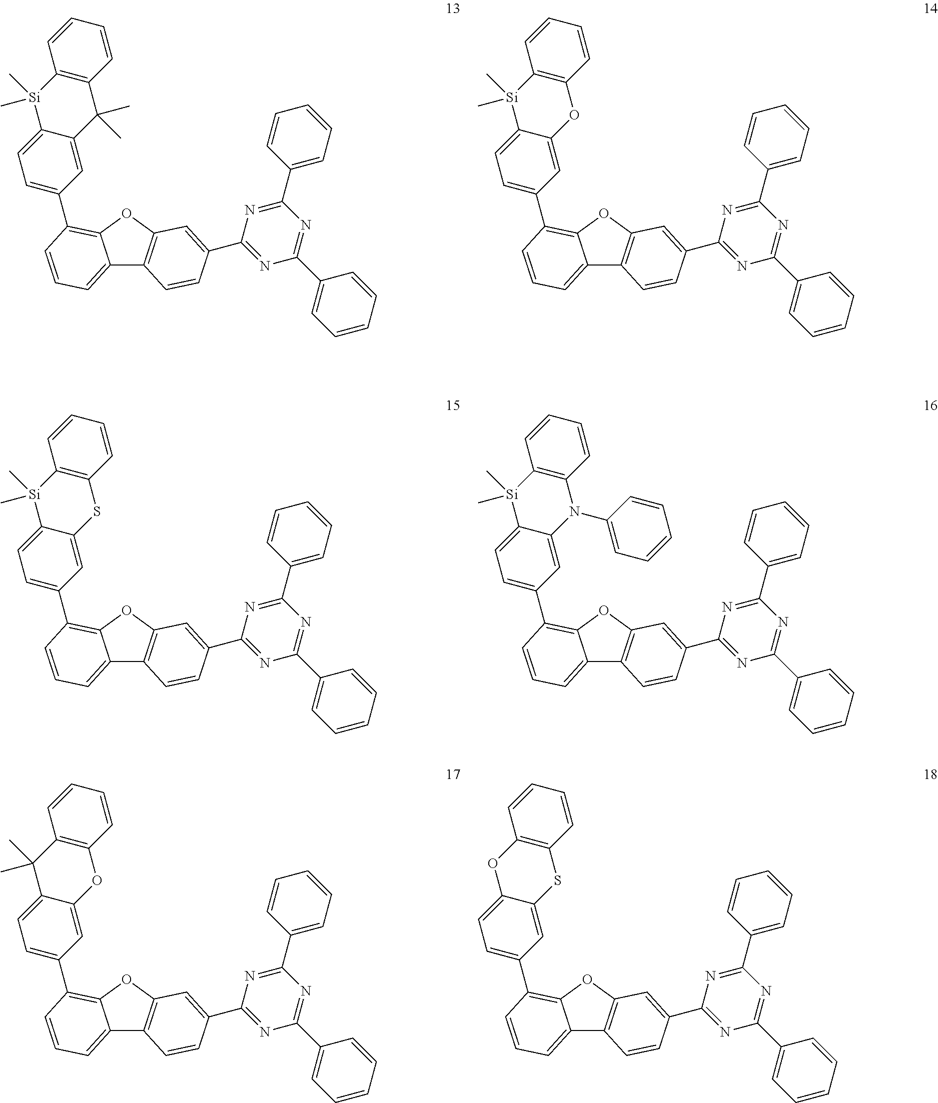

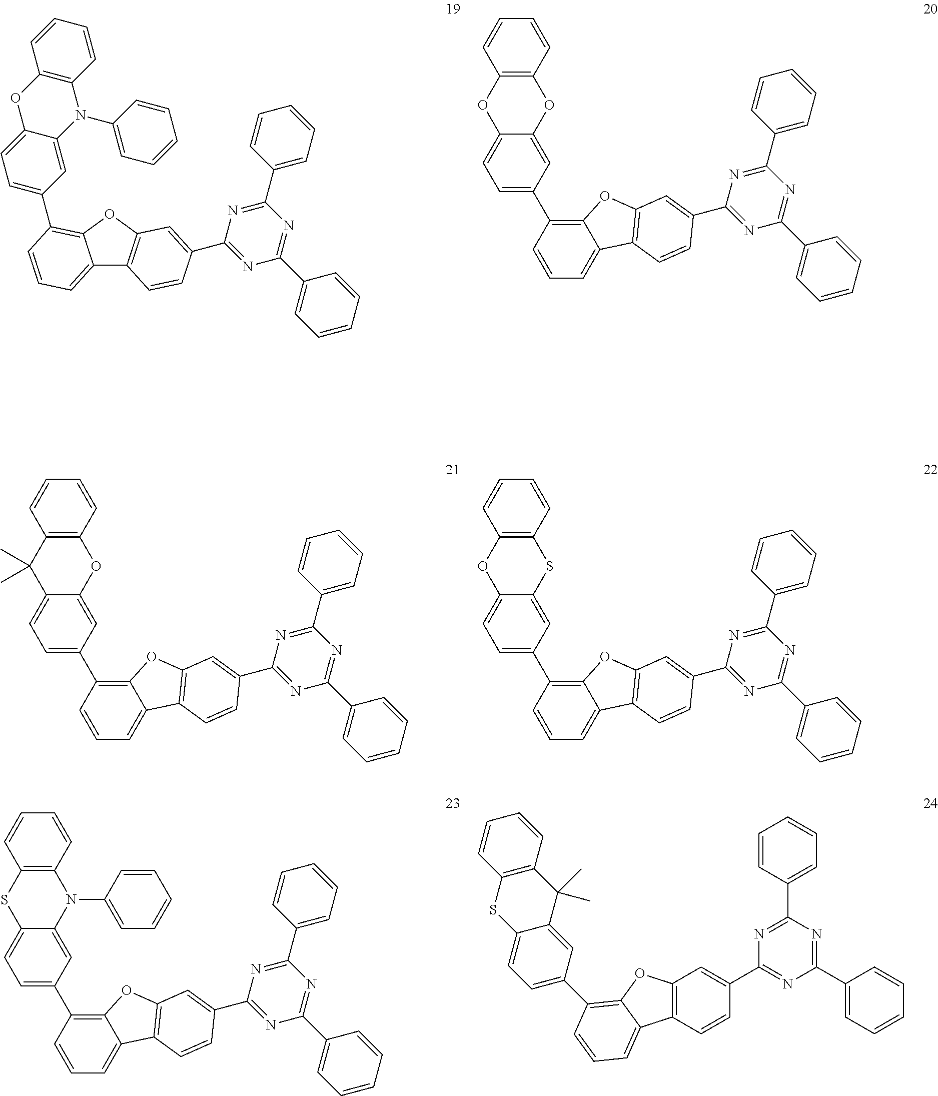

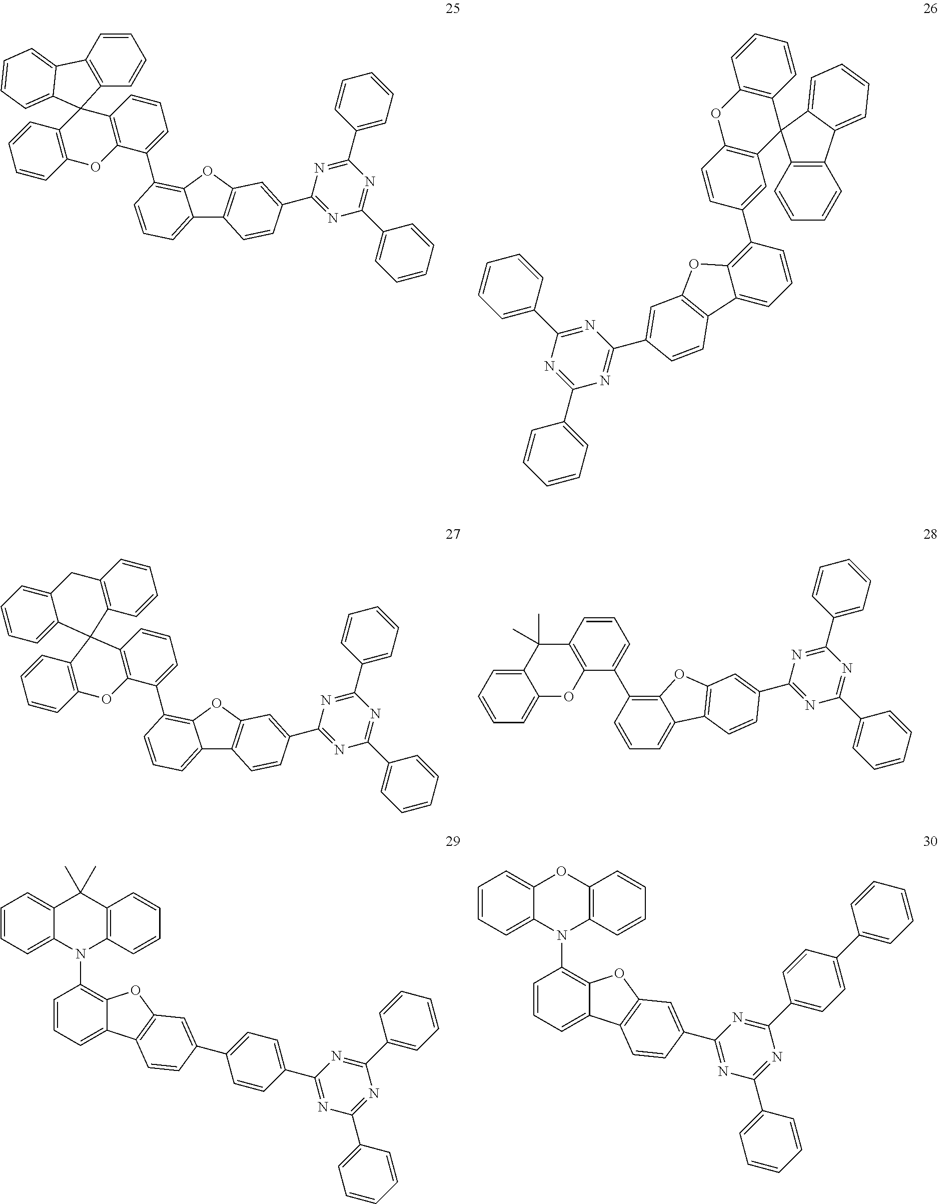

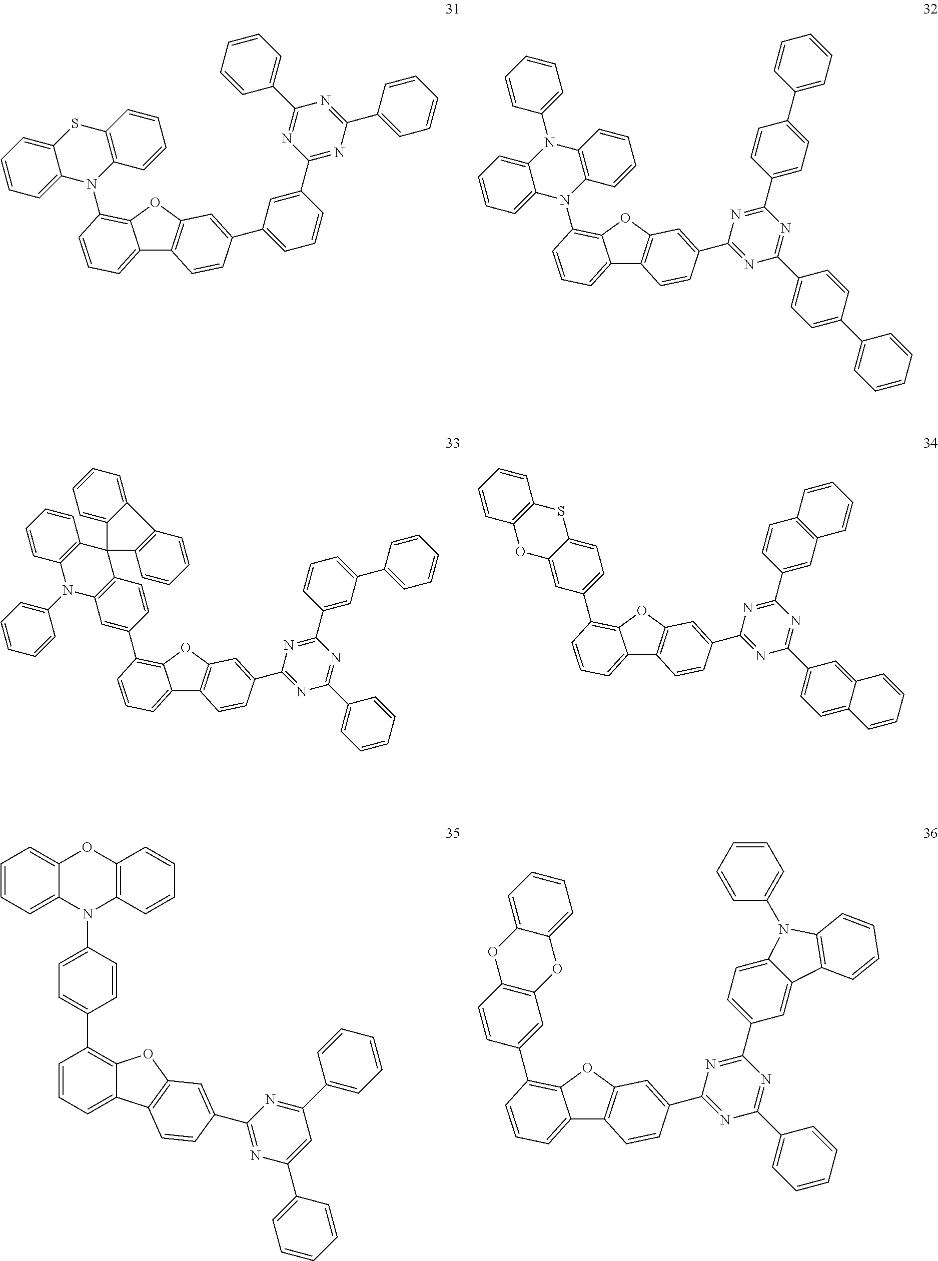

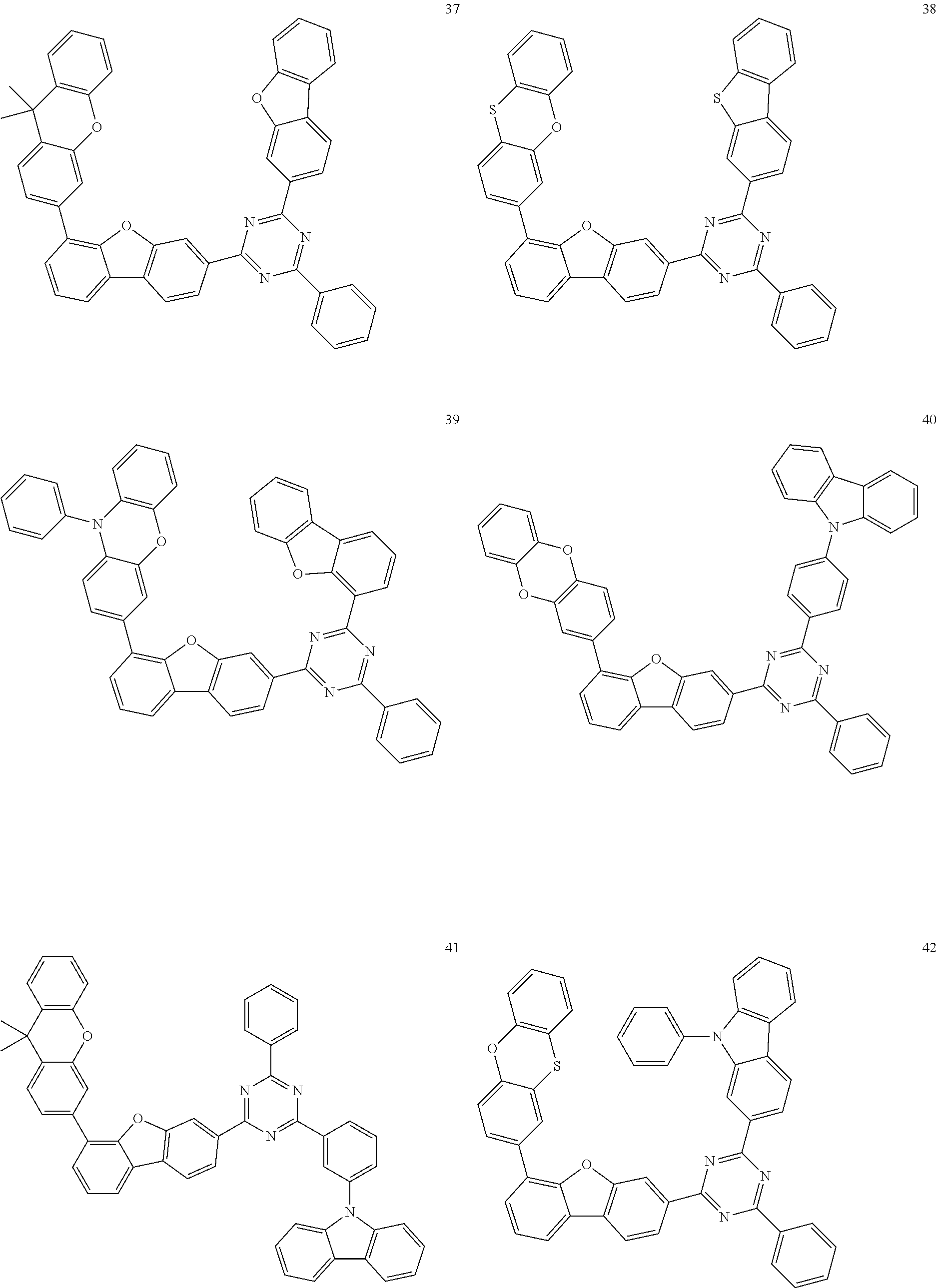

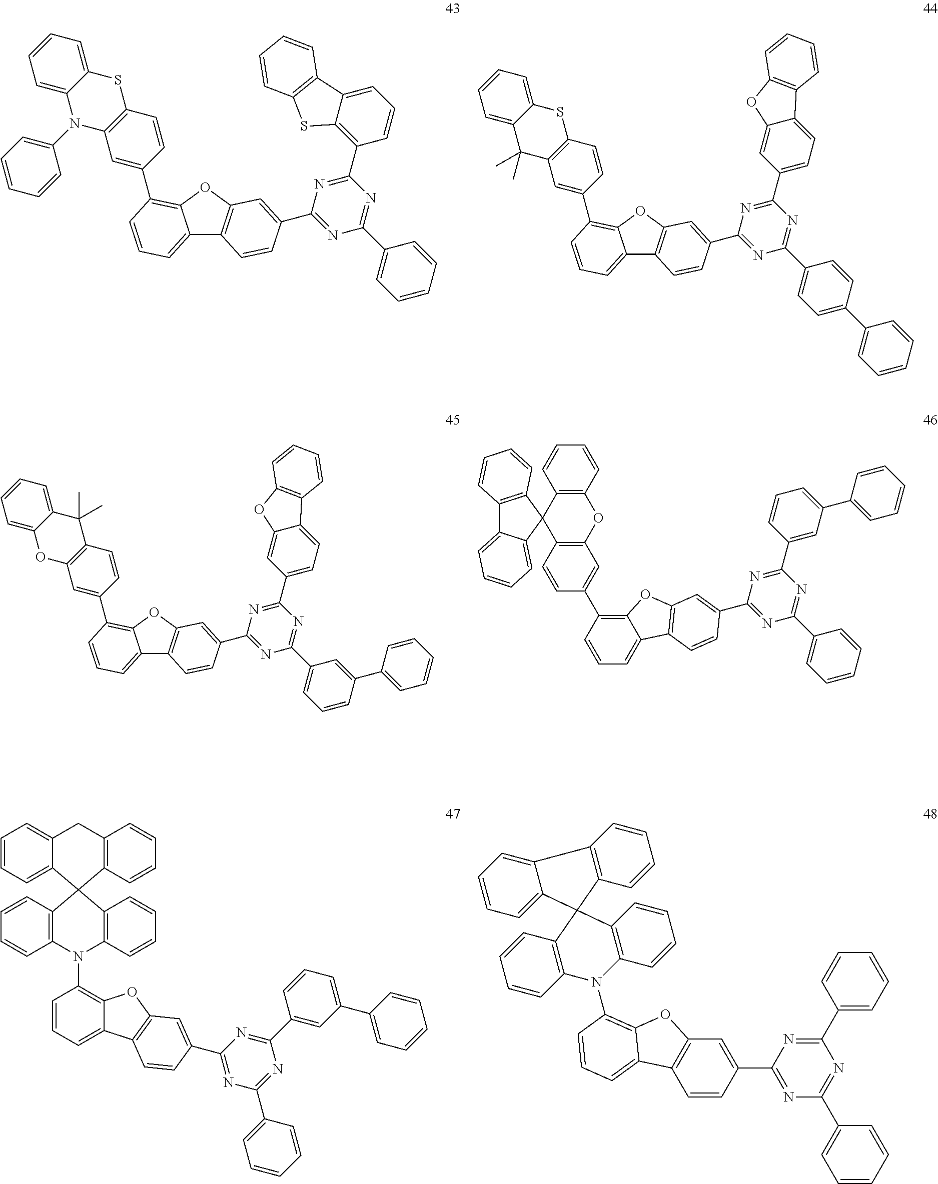

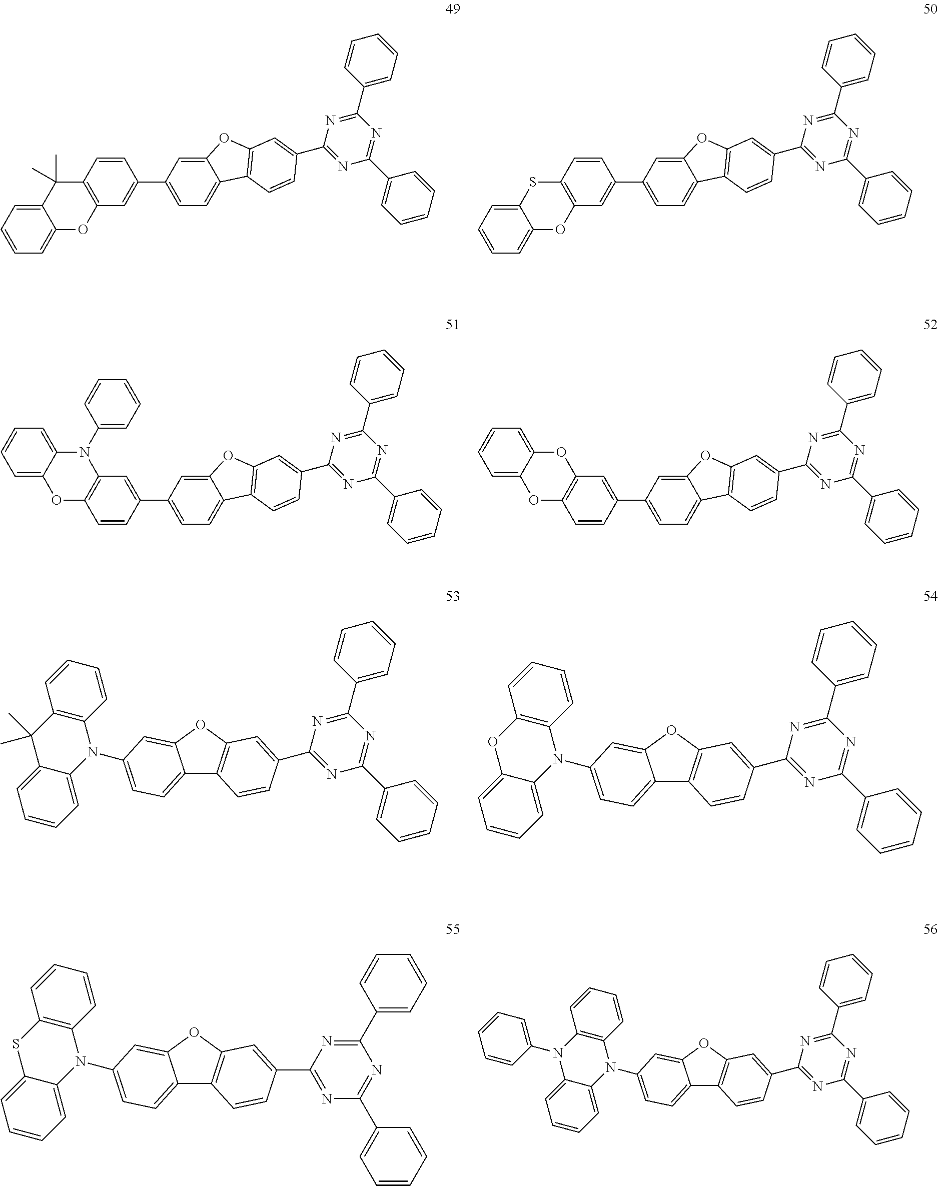

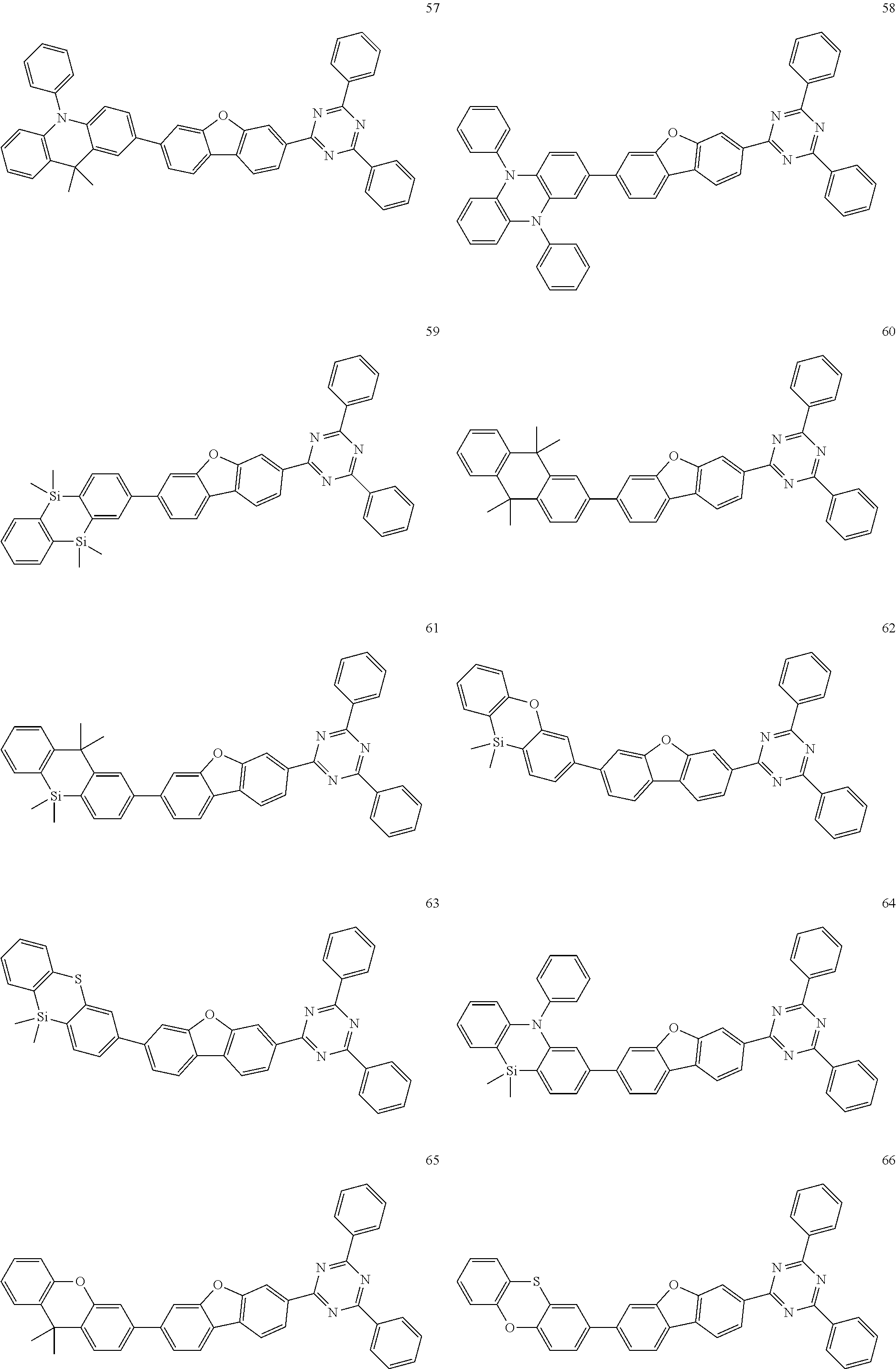

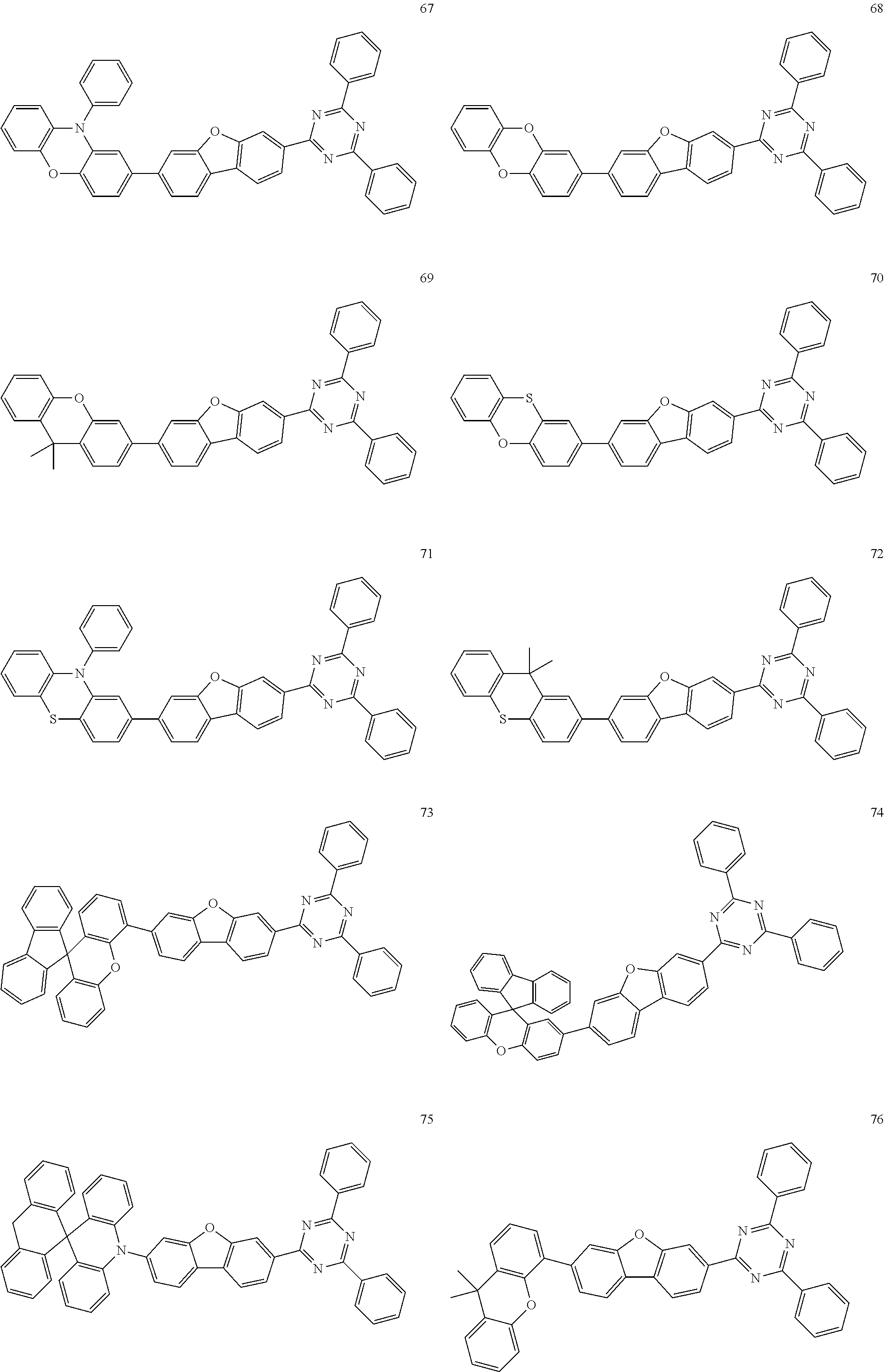

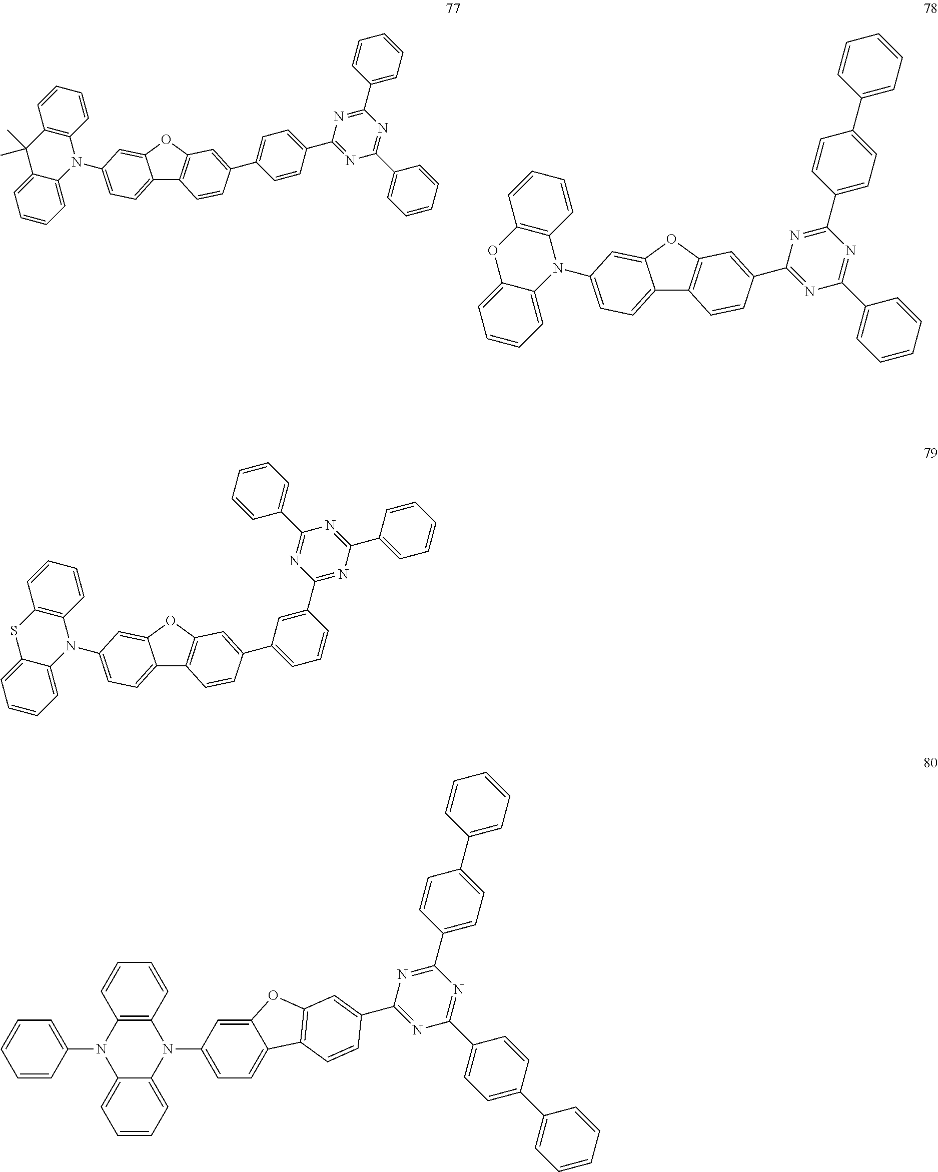 [SMILES: CC1(C)c2ccccc2N(c2ccc3c(c2)oc2cc(-c4ccc(-c5nc(-c6ccccc6)nc(-c6ccccc6)n5)cc4)ccc23)c2ccccc21 ; c1ccc(-c2ccc(-c3nc(-c4ccccc4)nc(-c4ccc5c(c4)oc4cc(N6c7ccccc7Oc7ccccc76)ccc45)n3)cc2)cc1 ; c1ccc(-c2nc(-c3ccccc3)nc(-c3cccc(-c4ccc5c(c4)oc4cc(N6c7ccccc7Sc7ccccc76)ccc45)c3)n2)cc1 ; c1ccc(-c2ccc(-c3nc(-c4ccc(-c5ccccc5)cc4)nc(-c4ccc5c(c4)oc4cc(N6c7ccccc7N(c7ccccc7)c7ccccc76)ccc45)n3)cc2)cc1]

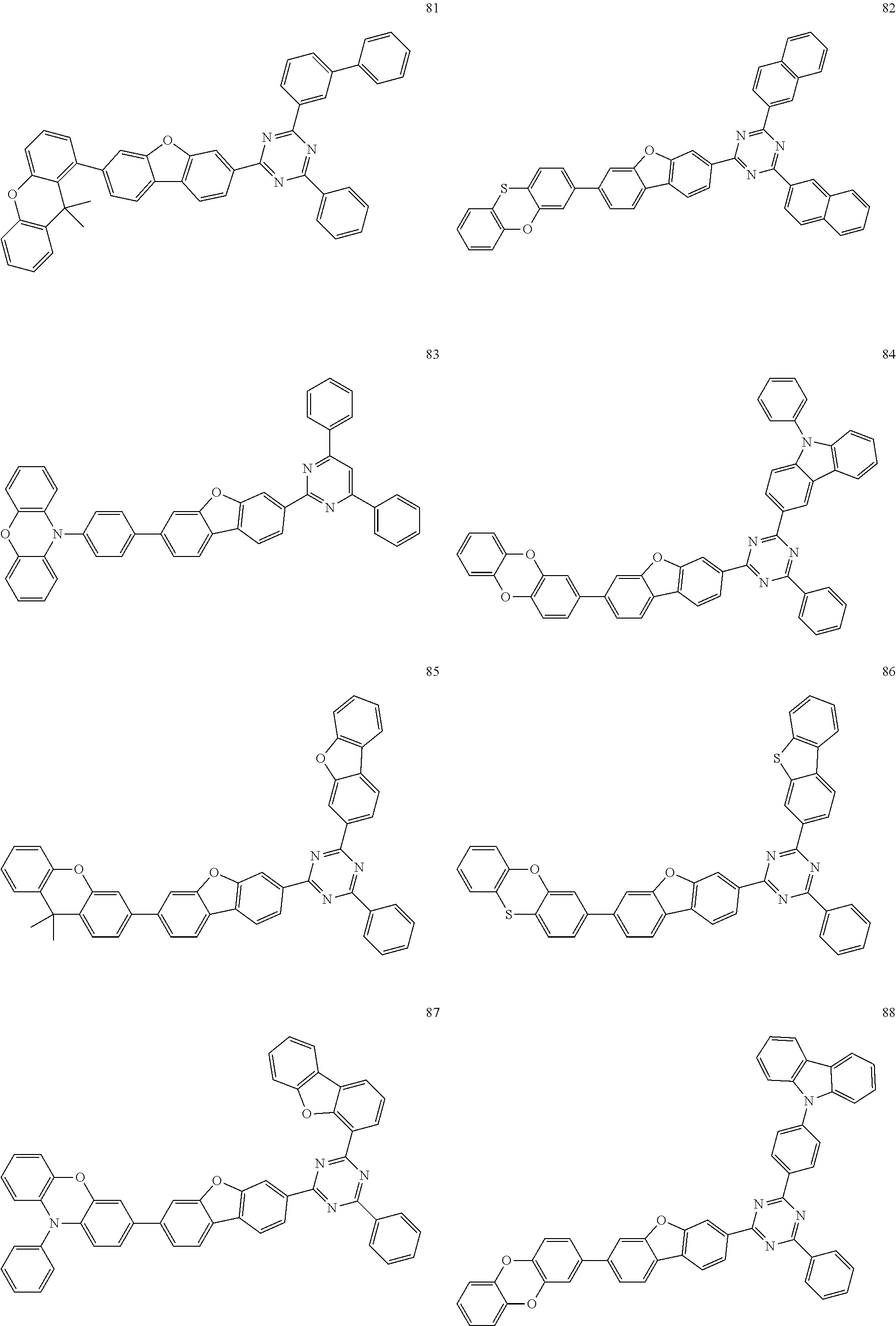

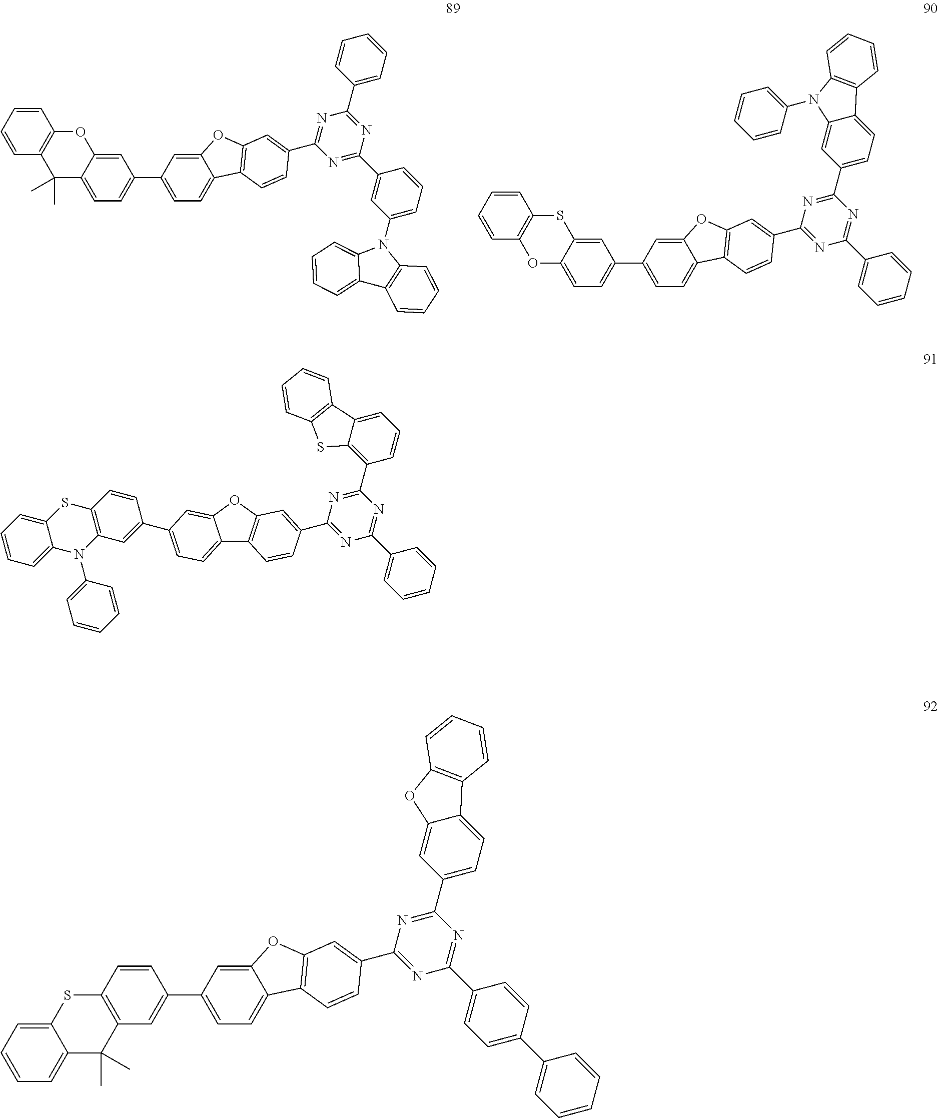 [SMILES: CC1(C)c2ccccc2Oc2cc(-c3ccc4c(c3)oc3cc(-c5nc(-c6ccccc6)nc(-c6cccc(-n7c8ccccc8c8ccccc87)c6)n5)ccc34)ccc21 ; c1ccc(-c2nc(-c3ccc4c(c3)oc3cc(-c5ccc6c(c5)Sc5ccccc5O6)ccc34)nc(-c3ccc4c5ccccc5n(-c5ccccc5)c4c3)n2)cc1 ; c1ccc(-c2nc(-c3ccc4c(c3)oc3cc(-c5ccc6c(c5)N(c5ccccc5)c5ccccc5S6)ccc34)nc(-c3cccc4c3sc3ccccc34)n2)cc1 ; CC1(C)c2ccccc2Sc2ccc(-c3ccc4c(c3)oc3cc(-c5nc(-c6ccc(-c7ccccc7)cc6)nc(-c6ccc7c(c6)oc6ccccc67)n5)ccc34)cc21]

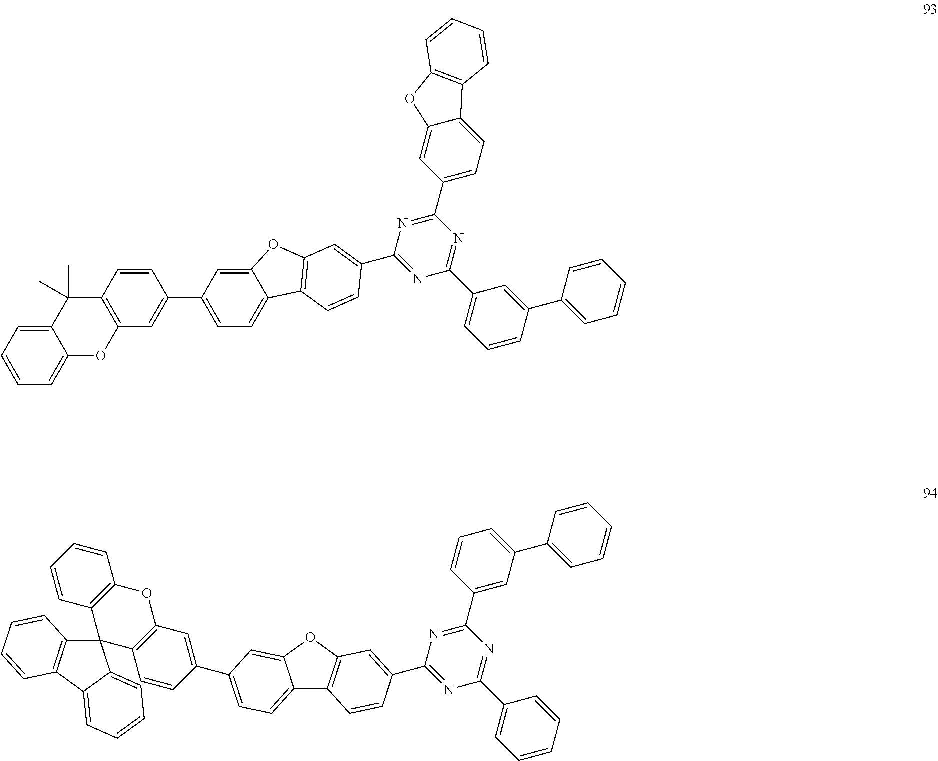 [SMILES: CC1(C)c2ccccc2Oc2cc(-c3ccc4c(c3)oc3cc(-c5nc(-c6cccc(-c7ccccc7)c6)nc(-c6ccc7c(c6)oc6ccccc67)n5)ccc34)ccc21 ; c1ccc(-c2cccc(-c3nc(-c4ccccc4)nc(-c4ccc5c(c4)oc4cc(-c6ccc7c(c6)Oc6ccccc6C76c7ccccc7-c7ccccc76)ccc45)n3)c2)cc1]

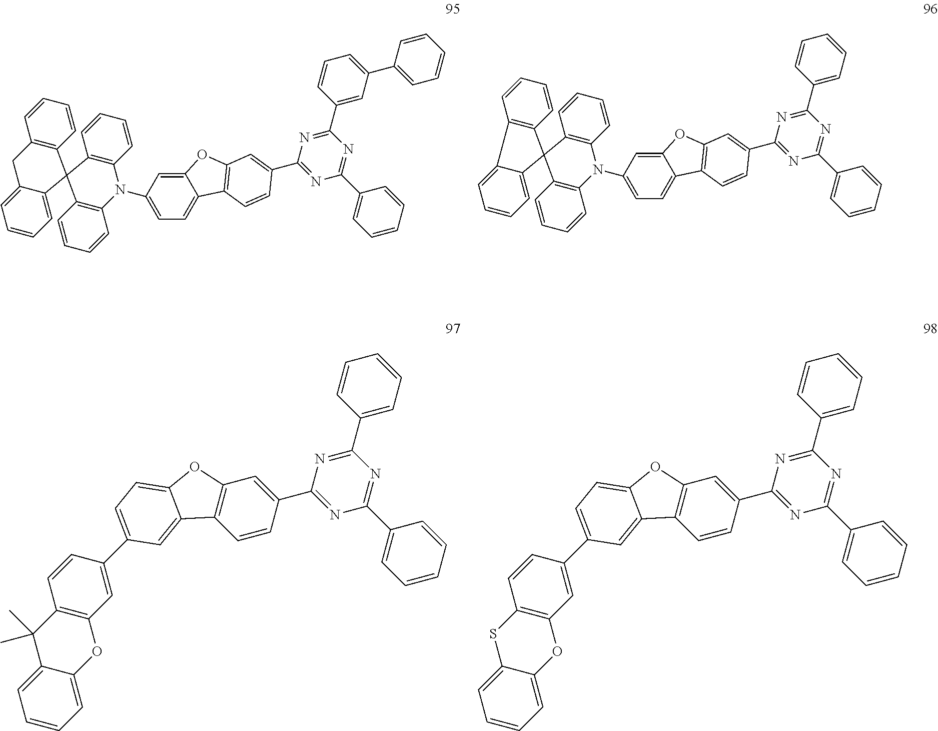 [SMILES: c1ccc(-c2cccc(-c3nc(-c4ccccc4)nc(-c4ccc5c(c4)oc4cc(N6c7ccccc7C7(c8ccccc8Cc8ccccc87)c7ccccc76)ccc45)n3)c2)cc1 ; c1ccc(-c2nc(-c3ccccc3)nc(-c3ccc4c(c3)oc3cc(N5c6ccccc6C6(c7ccccc7-c7ccccc76)c6ccccc65)ccc34)n2)cc1 ; CC1(C)c2ccccc2Oc2cc(-c3ccc4oc5cc(-c6nc(-c7ccccc7)nc(-c7ccccc7)n6)ccc5c4c3)ccc21 ; c1ccc(-c2nc(-c3ccccc3)nc(-c3ccc4c(c3)oc3ccc(-c5ccc6c(c5)Oc5ccccc5S6)cc34)n2)cc1]

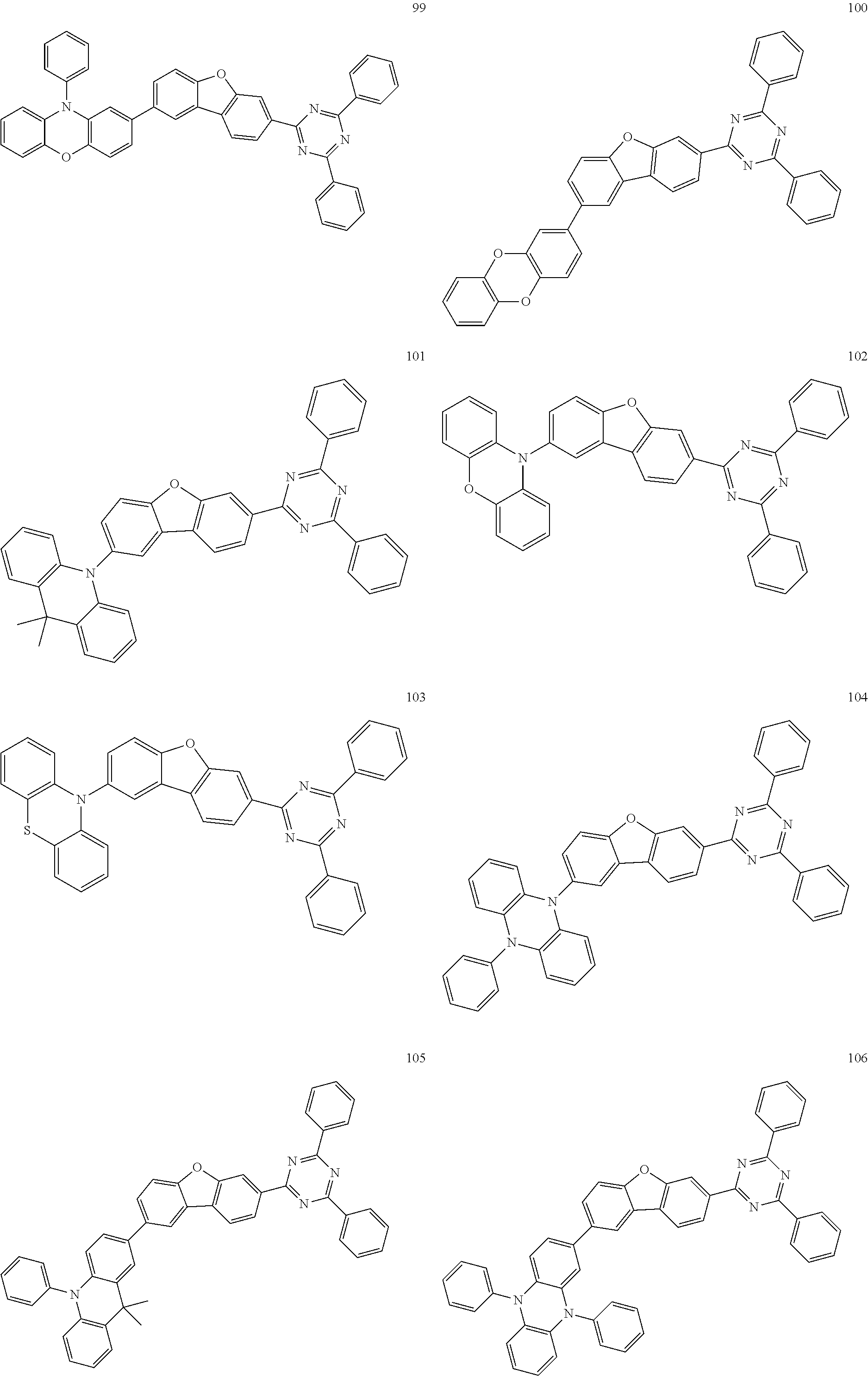

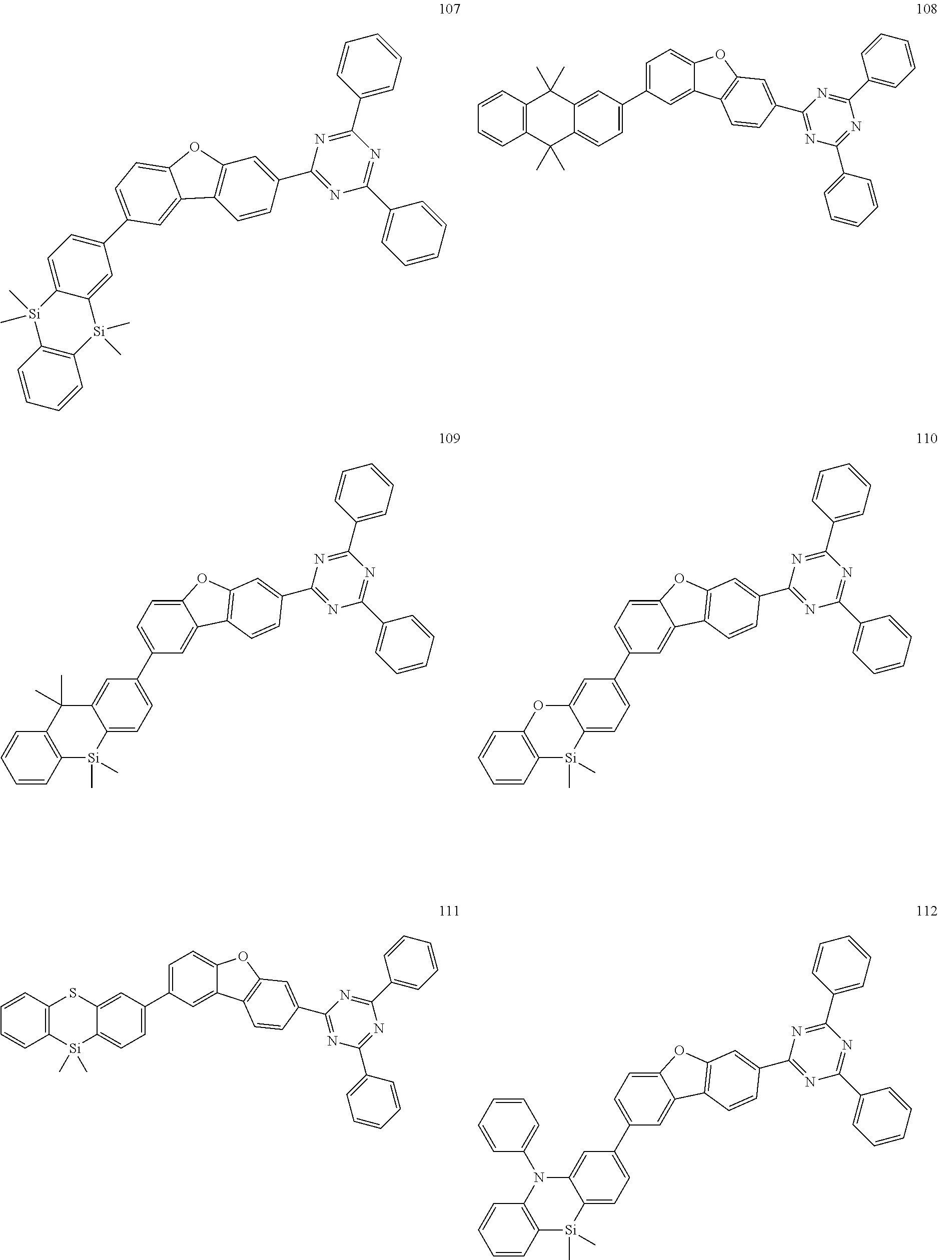

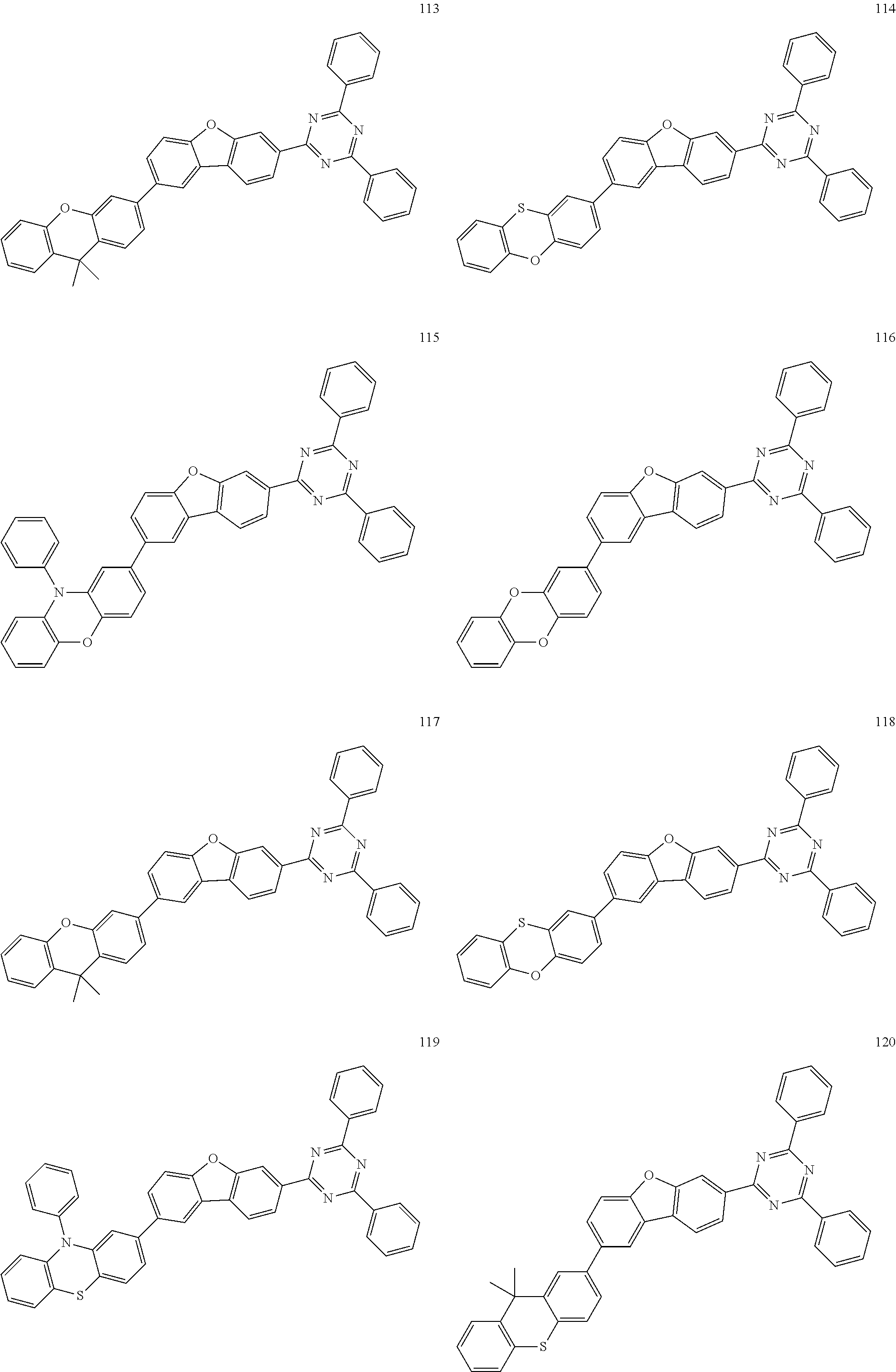

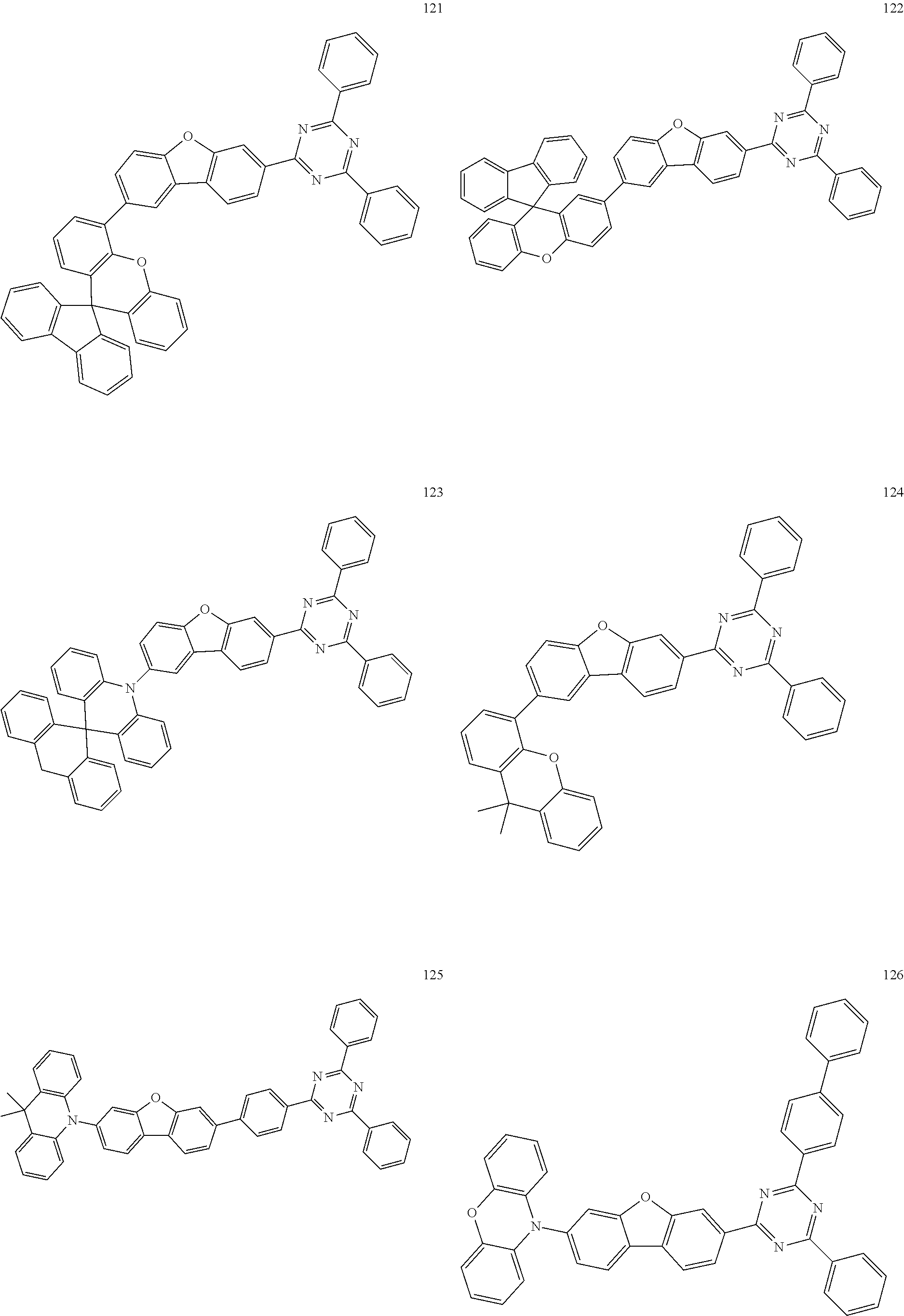

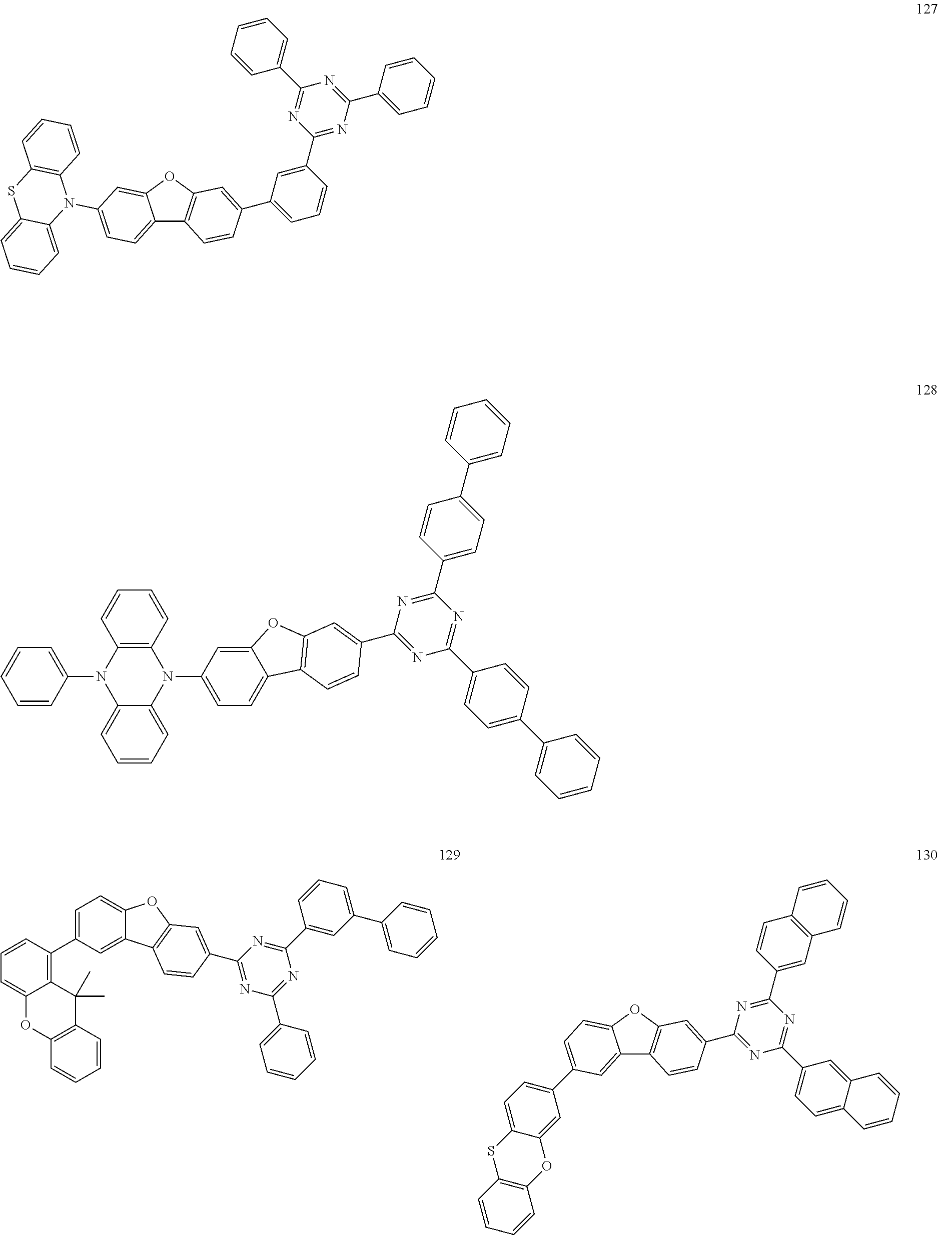 [SMILES: c1ccc(-c2nc(-c3ccccc3)nc(-c3cccc(-c4ccc5c(c4)oc4cc(N6c7ccccc7Sc7ccccc76)ccc45)c3)n2)cc1 ; c1ccc(-c2ccc(-c3nc(-c4ccc(-c5ccccc5)cc4)nc(-c4ccc5c(c4)oc4cc(N6c7ccccc7N(c7ccccc7)c7ccccc76)ccc45)n3)cc2)cc1 ; CC1(C)c2ccccc2Oc2cccc(-c3ccc4oc5cc(-c6nc(-c7ccccc7)nc(-c7cccc(-c8ccccc8)c7)n6)ccc5c4c3)c21 ; c1ccc2c(c1)Oc1cc(-c3ccc4oc5cc(-c6nc(-c7ccc8ccccc8c7)nc(-c7ccc8ccccc8c7)n6)ccc5c4c3)ccc1S2]

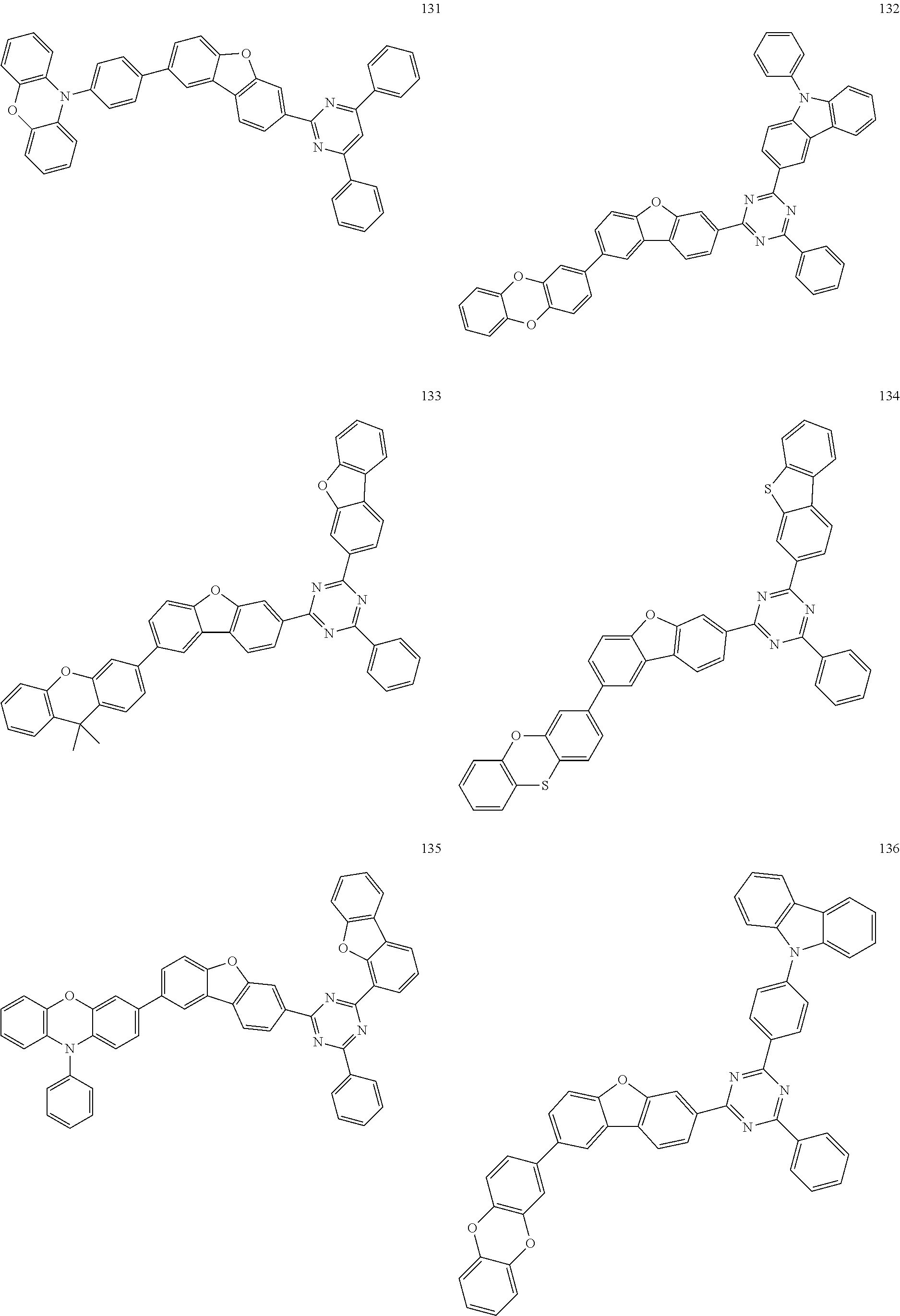

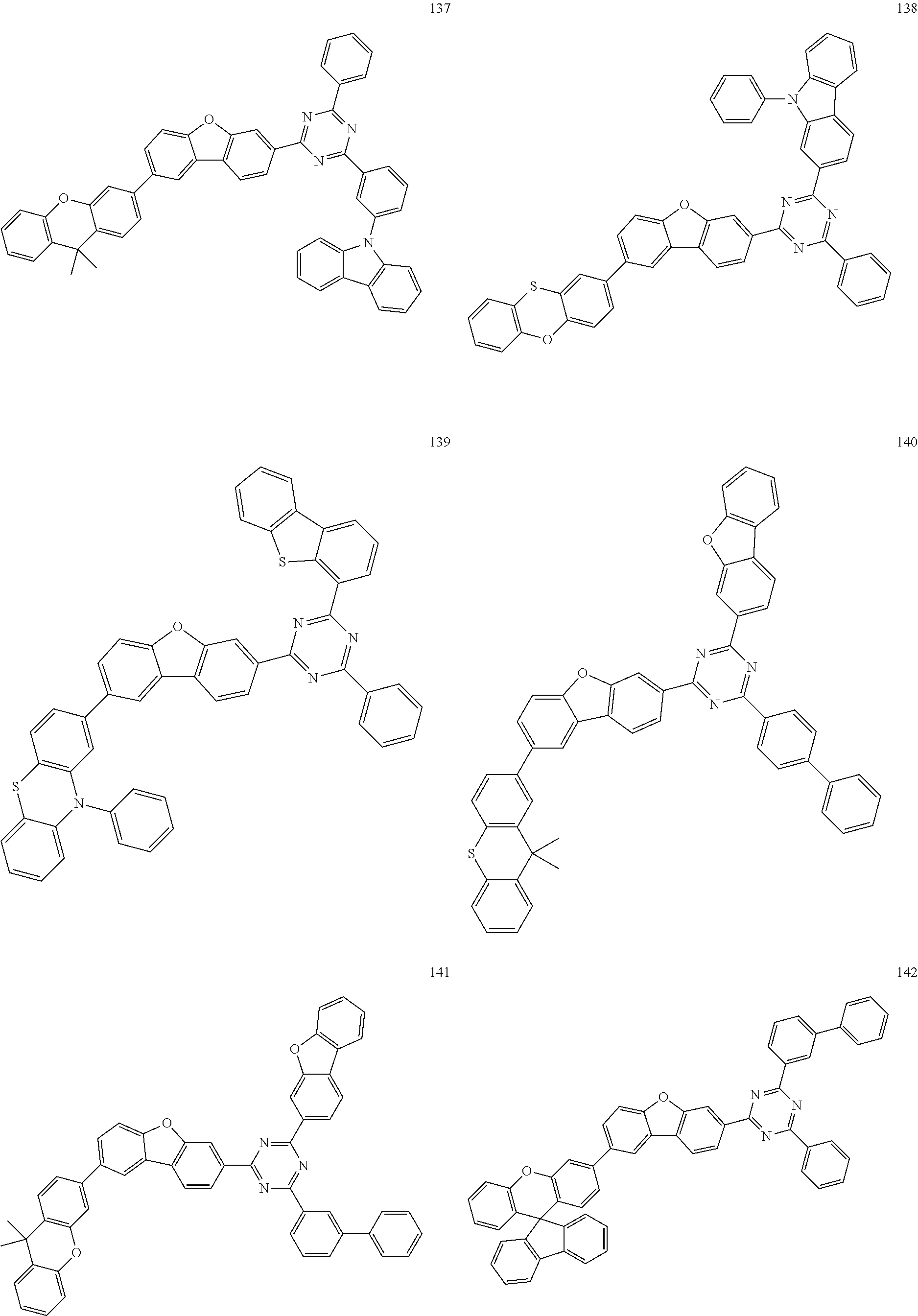

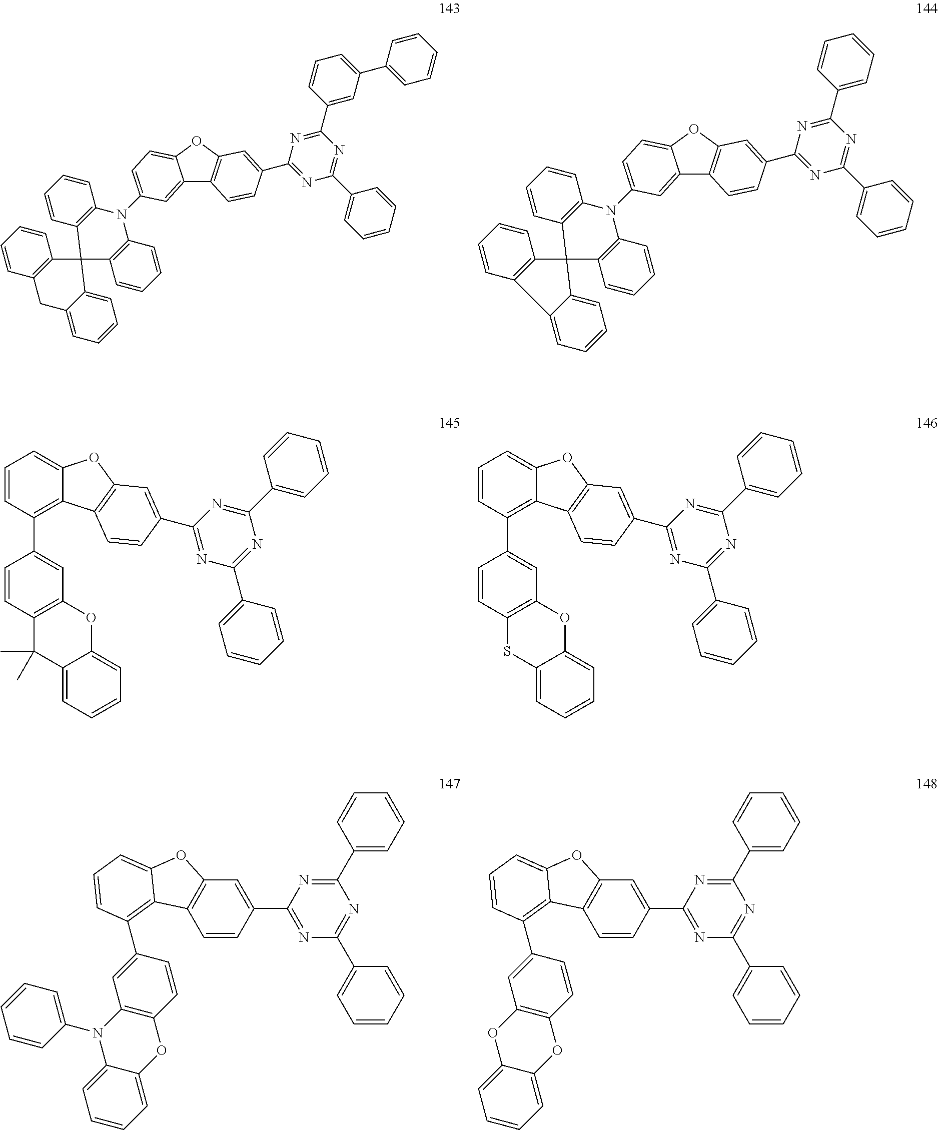

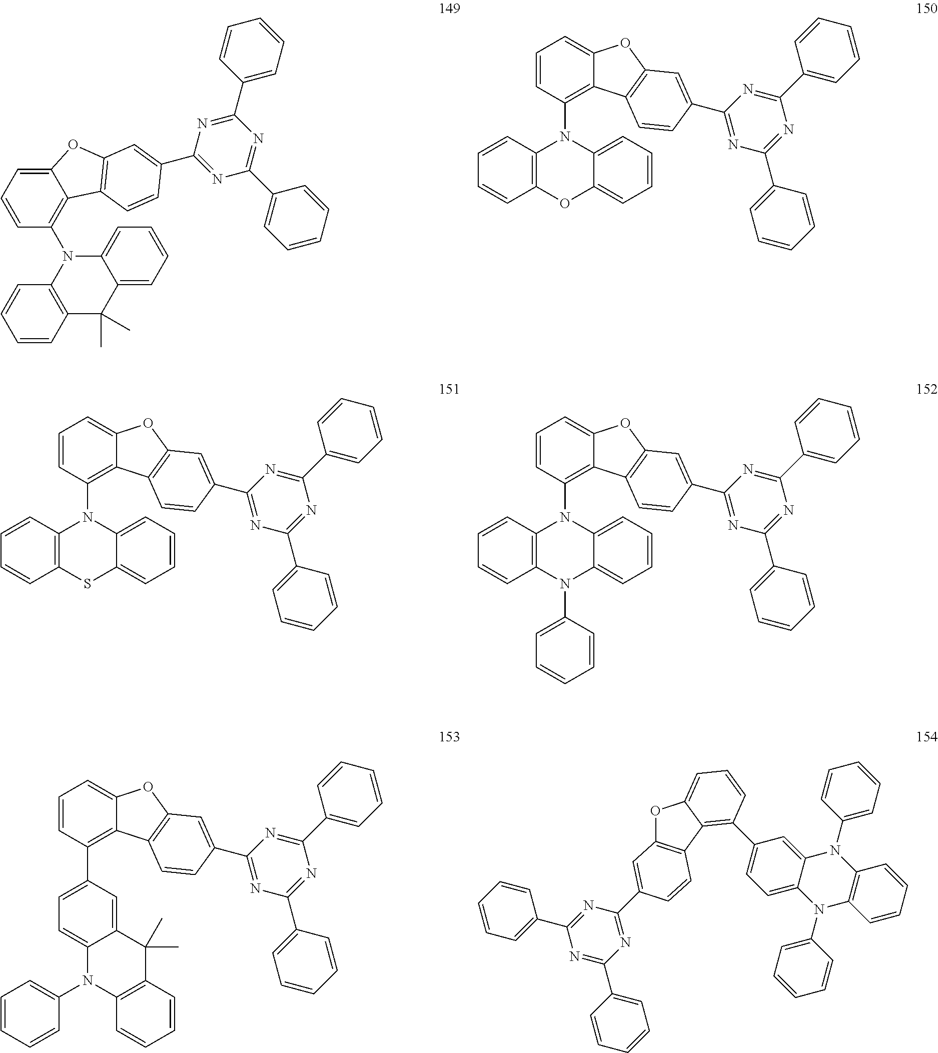

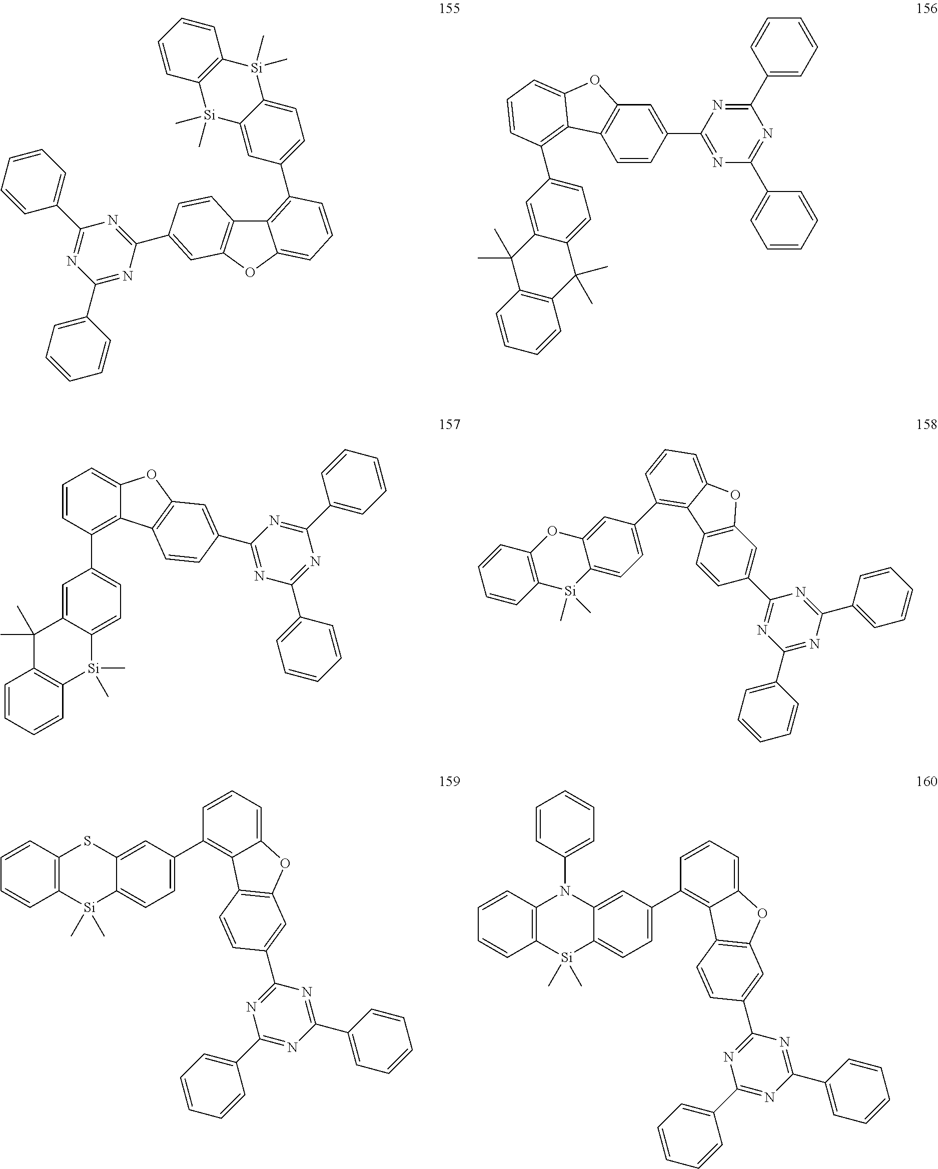

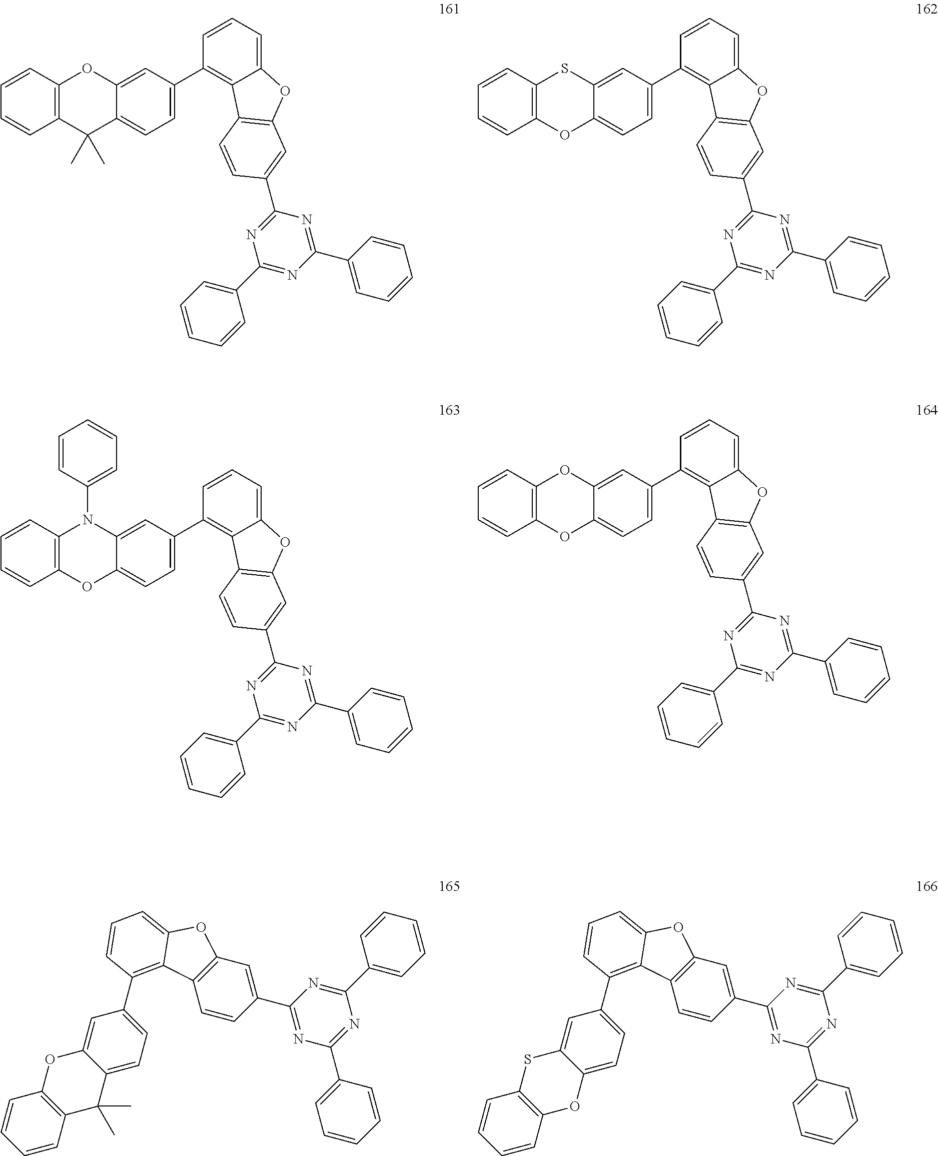 [SMILES: CC1(C)c2ccccc2Oc2cc(-c3cccc4oc5cc(-c6nc(-c7ccccc7)nc(-c7ccccc7)n6)ccc5c34)ccc21 ; c1ccc(-c2nc(-c3ccccc3)nc(-c3ccc4c(c3)oc3cccc(-c5ccc6c(c5)Sc5ccccc5O6)c34)n2)cc1 ; c1ccc(-c2nc(-c3ccccc3)nc(-c3ccc4c(c3)oc3cccc(-c5ccc6c(c5)N(c5ccccc5)c5ccccc5O6)c34)n2)cc1 ; c1ccc(-c2nc(-c3ccccc3)nc(-c3ccc4c(c3)oc3cccc(-c5ccc6c(c5)Oc5ccccc5O6)c34)n2)cc1 ; CC1(C)c2ccccc2Oc2cc(-c3cccc4oc5cc(-c6nc(-c7ccccc7)nc(-c7ccccc7)n6)ccc5c34)ccc21 ; c1ccc(-c2nc(-c3ccccc3)nc(-c3ccc4c(c3)oc3cccc(-c5ccc6c(c5)Sc5ccccc5O6)c34)n2)cc1]

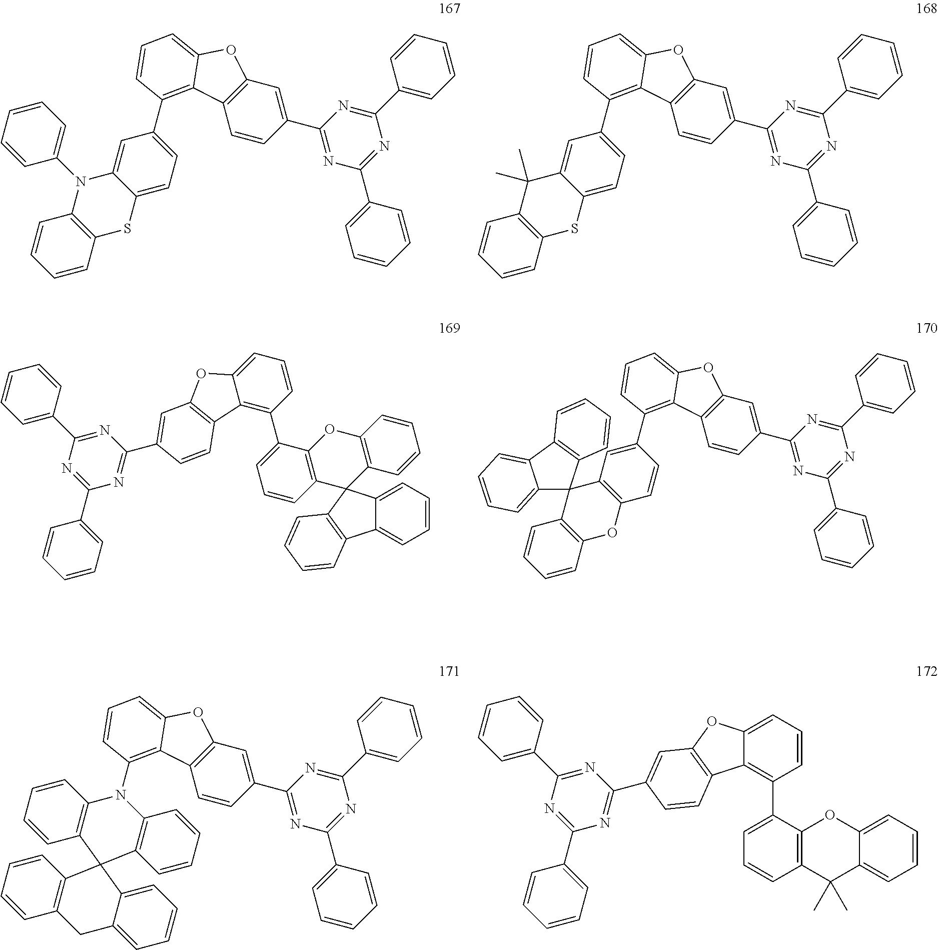

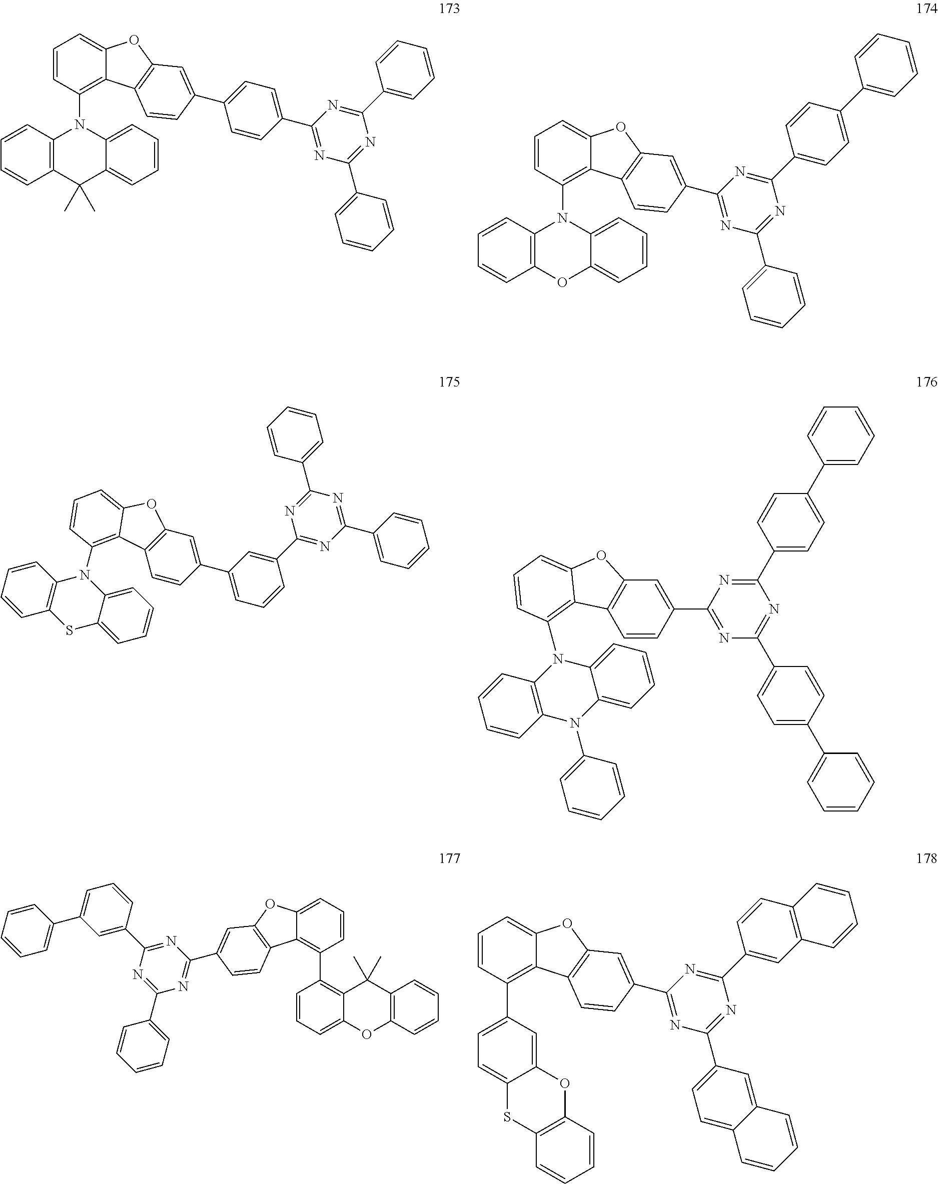

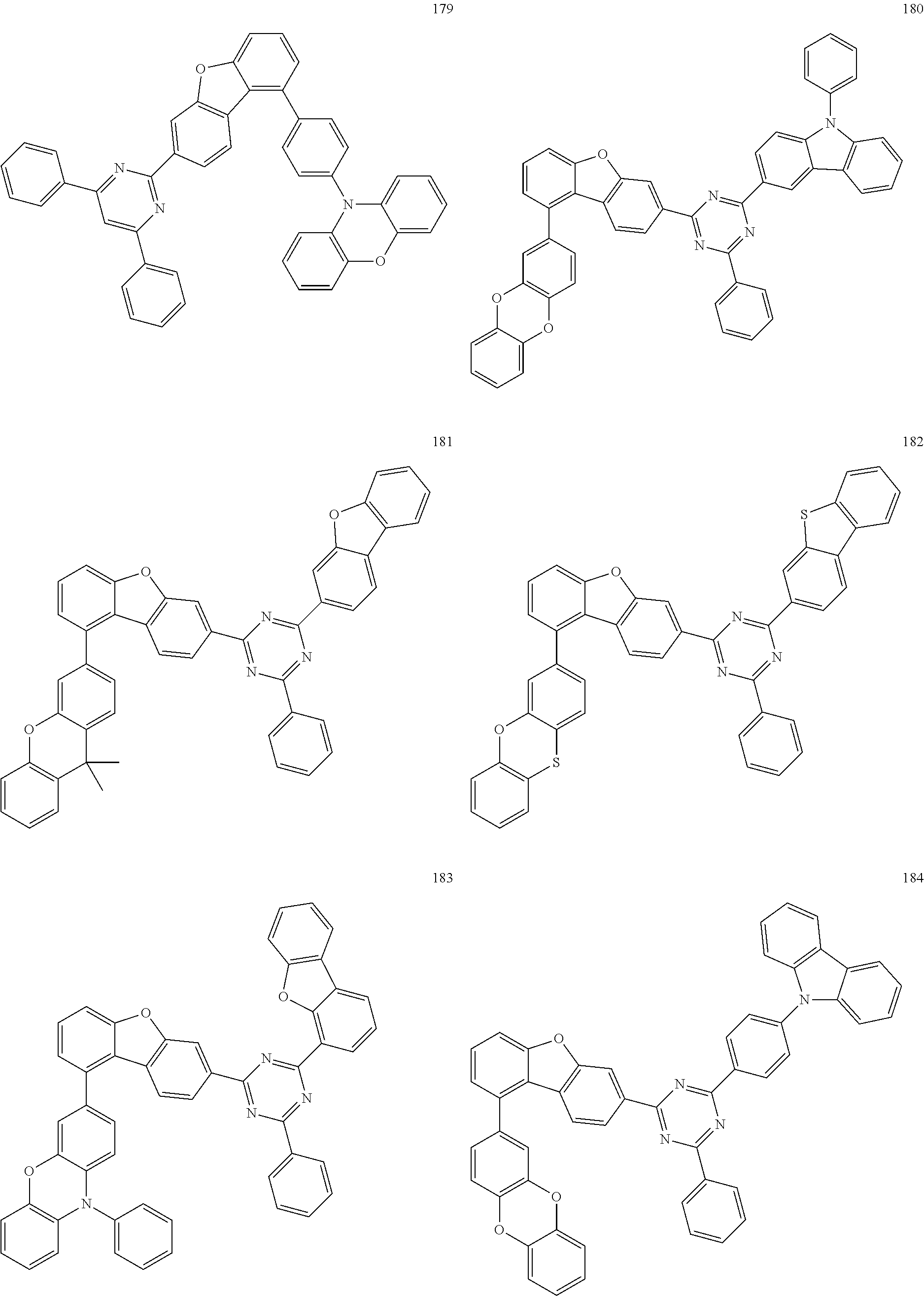

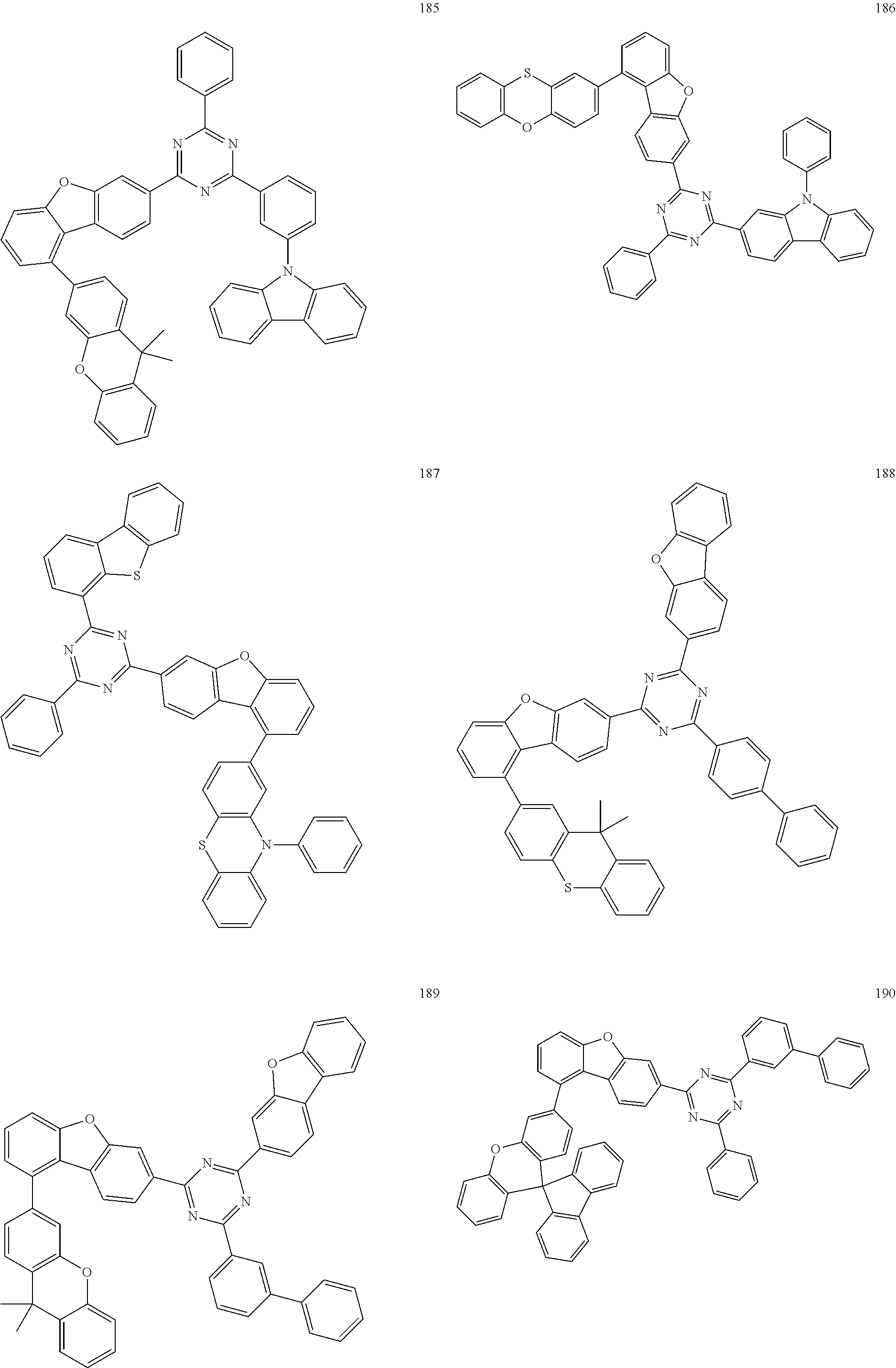

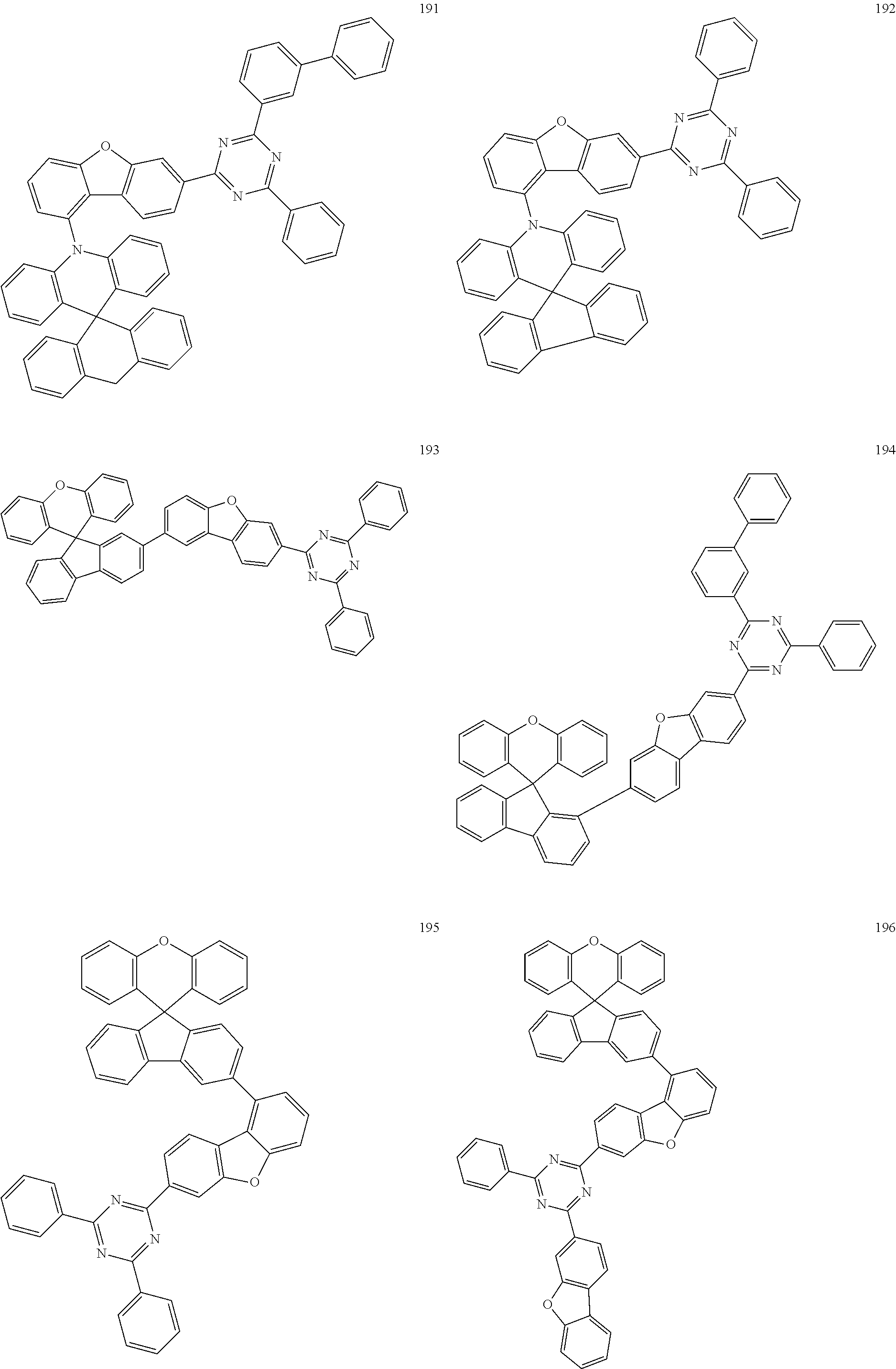

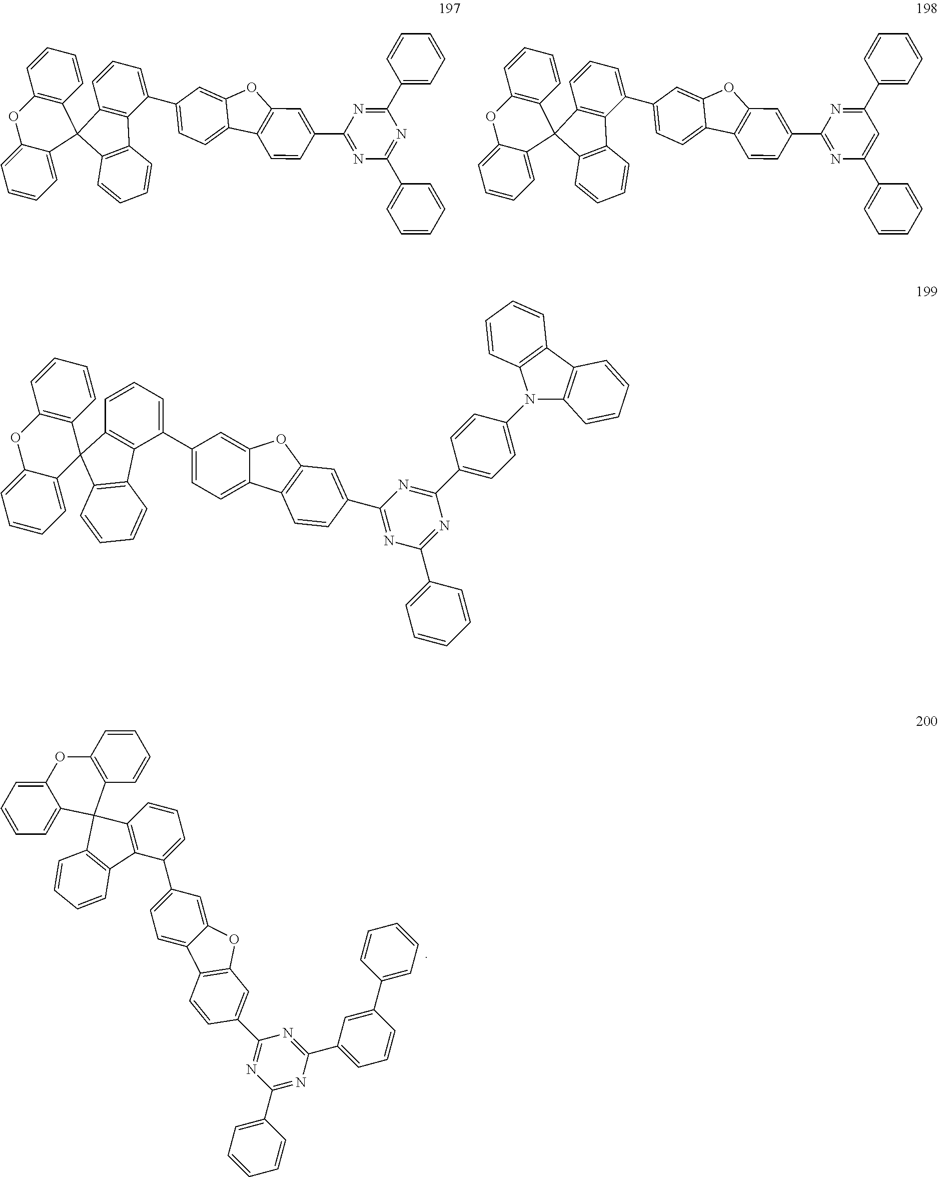 [SMILES: c1ccc(-c2nc(-c3ccccc3)nc(-c3ccc4c(c3)oc3cc(-c5cccc6c5-c5ccccc5C65c6ccccc6Oc6ccccc65)ccc34)n2)cc1 ; c1ccc(-c2cc(-c3ccccc3)nc(-c3ccc4c(c3)oc3cc(-c5cccc6c5-c5ccccc5C65c6ccccc6Oc6ccccc65)ccc34)n2)cc1 ; c1ccc(-c2nc(-c3ccc(-n4c5ccccc5c5ccccc54)cc3)nc(-c3ccc4c(c3)oc3cc(-c5cccc6c5-c5ccccc5C65c6ccccc6Oc6ccccc65)ccc34)n2)cc1 ; c1ccc(-c2cccc(-c3nc(-c4ccccc4)nc(-c4ccc5c(c4)oc4cc(-c6cccc7c6-c6ccccc6C76c7ccccc7Oc7ccccc76)ccc45)n3)c2)cc1]

7. An organic light emitting device comprising:
a first electrode;
a second electrode provided opposite to the first electrode; and
one or more organic material layers provided between the first electrode and the second electrode,
wherein one or more layers of the organic material layers comprise the heterocyclic compound of claim 1.

8. The organic light emitting device of claim 7, wherein the organic material layer comprises a light emitting layer, and the light emitting layer comprises the heterocyclic compound.

9. The organic light emitting device of claim 7, wherein the organic material layer comprises a light emitting layer, the light emitting layer comprises a host material, and the host material comprises the heterocyclic compound.

10. The organic light emitting device of claim 7, wherein the organic material layer comprises an electron injection layer or an electron transfer layer, and the electron transfer layer or the electron injection layer comprises the heterocyclic compound.

11. The organic light emitting device of claim 7, wherein the organic material layer comprises an electron blocking layer or a hole blocking layer, and the electron blocking layer or the hole blocking layer comprises the heterocyclic compound.

12. The organic light emitting device of claim 7, further comprising one, two or more layers selected from the group consisting of a light emitting layer, a hole injection layer, a hole transfer layer, an electron injection layer, an electron transfer layer, an electron blocking layer and a hole blocking layer.
